(19) United States
(12) Patent Application Publication
(10) Pub. No.: US 2024/0255495 A1
(43) Pub. Date: Aug. 1, 2024

(54) COVALENT APTAMERS

(71) Applicant: UNIVERSITY OF PITTSBURGH-OF THE COMMONWEALTH SYSTEM OF HIGHER EDUCATION, Pittsburgh, PA (US)

(72) Inventors: Alexander DEITERS, Pittsburgh, PA (US); Yaniv TIVON, Pittsburgh, PA (US); Mary CACACE, Pittsburgh, PA (US); Savannah ALBRIGHT, Pittsburgh, PA (US)

(21) Appl. No.: 18/557,516

(22) PCT Filed: Apr. 26, 2022
(86) PCT No.: PCT/US2022/026414

§ 371 Date: Oct. 26, 2023

Related U.S. Application Data
(60) Provisional application No. 63/179,911, filed on Apr. 26, 2021.

(51) Int. Cl. G01N 33/53; C12N 15/10; C12N 15/115; G01N 33/569; G01N 33/573; G01N 33/574; G01N 33/68

(52) U.S. Cl. CPC G01N 33/5308; C12N 15/1048; C12N 15/115; G01N 33/56983; G01N 33/573; G01N 33/57488; G01N 33/6851; C12N 2310/16; C12N 2310/314; C12N 2310/3511; C12N 2310/3517; C12N 2310/531; G01N 2333/165; G01N 2333/912; G01N 2333/974; G01N 2469/20

(57) ABSTRACT

Disclosed are chemically modified aptamers comprising an electrophilic group and a handle and methods for using said aptamers to deliver the handle to a target protein, crosslink the aptamer to the target protein, as well as methods of using said aptamers to detect proteins and to treat viral infections or cancer.

This application claims the benefit of U.S. Provisional Application No. 63/179,911, filed on Apr. 26, 2021, which is incorporated herein by reference in its entirety.

This invention was made with government support under Grant No. AI130815 awarded by the National Institutes of Health. The government has certain rights in the invention.

### I. BACKGROUND

Covalent modification of proteins via bioorthogonal reactions is an important and powerful tool for biological studies and the development of therapeutics. Some common approaches to generate protein conjugates in complex biological settings include metabolic labeling, activity-based probes, fusion of the protein of interest to a self-labeling enzyme (e.g., SNAPtag), addition of a peptide tag (e.g., Sortag), and incorporation of unnatural amino acids. Targeted covalent modification of native proteins, however, remains challenging. What are needed are new methods for modifying proteins and detecting protein targets.

### II. SUMMARY

Disclosed are methods and compositions related to “covalent aptamers”, engineered, nucleic acid-based protein ligands that either covalently transfer a handle or covalently crosslink to their target.

In one aspect, disclosed herein are modified aptamer-handle conjugates comprising an aptamer (including, but not limited to the aptamers as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and/or SEQ ID NO: 20), an electrophilic leaving group (such as, for example, N-acyl sulfonamide cleavable electrophile or tosyl cleavable electrophile) and a handle (including, but not limited to a biotin, a bioconjugation handle, a chemiluminescent marker, a fluorescent marker, radiomarker, dye, quantum dot, enzyme, enzyme substrate, catalyst, small molecule ligand, PROTAC® (E3 ligase ligand), or LYTAC® (cation-independent mannose-6-phosphate receptor (CI-M6PR) ligand); wherein the handle is bound to the electrophile and the electrophile is bound to the aptamer. In some aspects, the electrophile is bound to the aptamer via an aryl sulfonamide on the electrophile. In some aspects the electrophile is inverted and bound to the aptamer via an amide motif on the electrophile, leading to aptamer-protein crosslinking instead of handle transfer. For example, in one aspect disclosed herein are modified aptamer-handle conjugates wherein the electrophile comprises an N-acyl sulfonamide; and wherein the N-acyl sulfonamide is connected to the aptamer via the non-cleavable benzamide nitrogen, any non-cleavable connection to the aryl ring of the sulfonamide group, or via the carbonyl group of the cleavable amide. In some aspects disclosed herein are modified aptamer-handle conjugates wherein the electrophile comprises a tosyl electrophile; and wherein the tosyl electrophile is connected to the aptamer via the non-cleavable benzamide nitrogen, via any non-cleavable connection to the aryl ring of the tosylate group, or via the alpha-CH₂ carbon of the cleavable tosylate moiety.

In one aspect, disclosed herein are modified aptamers (including, but not limited to the aptamers as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and/or SEQ ID NO: 20) comprising an electrophilic leaving group (such as, for example, N-acyl sulfonamide cleavable electrophile or tosyl cleavable electrophile) for crosslinking to a target. In some aspects, the electrophile is bound to the aptamer via an aryl sulfonamide on the electrophile. In some aspects the electrophile is inverted and bound to the aptamer via an amide motif on the electrophile, leading to aptamer-protein crosslinking instead of handle transfer. For example, in one aspect disclosed herein are modified aptamers wherein the electrophile comprises an N-acyl sulfonamide; and wherein the N-acyl sulfonamide is connected to the aptamer via the non-cleavable benzamide nitrogen, any non-cleavable connection to the aryl ring of the sulfonamide group, or via the carbonyl group of the cleavable amide. In some aspects disclosed herein are modified aptamers wherein the electrophile comprises a tosyl electrophile; and wherein the tosyl electrophile is connected to the aptamer via the non-cleavable benzamide nitrogen, via any non-cleavable connection to the aryl ring of the tosylate group, or via the alpha-CH₂ carbon of the cleavable tosylate moiety. In some aspects, the aptamer can comprise a detectable moiety including, but not limited to a label (including biotins, a chemiluminescent marker, a fluorescent marker (e.g., fluorophores), radiomarkers, organocatalysts, dyes, quantum dots, enzymes, enzyme substrates, and other detectable markers), metal catalysts, small molecule ligands, PROTAC® (E3 ligase ligands), or LYTAC® (cation-independent mannose-6-phosphate receptor (CI-M6PR) ligands), drugs, or small molecule inhibitors.

Also disclosed herein are methods for the diagnosis and/or detection of a specific disease state in a tissue, fluid, or cell sample (including, but not limited to fresh and/or fixed samples such as, for example, formalin fixed paraffin embedded (FFPE) samples) comprising: (a) contacting a tissue sample or cell sample with an aptamer, wherein the aptamer comprises at least one aptamer of any preceding aspect; (b) measuring the presence, absence or quantity of the aptamer or the transferred handle; and (c) diagnosing the disease state of the tissue or cell based on the presence, absence or quantity of the aptamer measured. For example, disclosed herein are methods of diagnosing and/or detecting a coronavirus infection (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) comprising contacting a tissue sample or cell sample with the modified aptamer handle conjugates of any preceding aspect, wherein the aptamer selectively binds the coronal virus spike protein or the receptor binding domain of the coronavirus spike or nucleocapsid protein (such as, for example, the aptamer as set forth in SEQ ID NO: 9, SEQ ID NO: 10, SEQ ID NO: 11, SEQ ID NO: 12, SEQ ID NO: 13, SEQ ID NO: 14, and/or SEQ ID NO: 15); (b) measuring the presence, absence or quantity of the aptamer or the transferred handle; and (c) diagnosing and/or detecting the a coronavirus infection based on the presence, absence or quantity of the aptamer or transferred handle measured. Also for example, disclosed herein are methods of diagnosing and/or detecting a circulatory condition (such as, for example, thrombosis, thromboembolism, Paget-Schroetter disease, fibrosis, stroke, or myocardial infarction) in a subject comprising contacting a tissue sample or cell sample with the modified aptamer handle conjugate of any preceding aspect, wherein the aptamer selectively binds and covalently modifies thrombin, including, but not limited to, an aptamer as set forth in SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8) (b) measuring the presence, absence or quantity of the aptamer; and (c) diagnosing and/or detecting the disease state of the tissue or cell based on the presence, absence or quantity of the aptamer measured. Also for example, disclosed herein are methods of diagnosing and/or detecting a cancer and/or metastasis (such as, for example, retinoblastoma, colon cancer, lung cancer, and/or gastric cancers) in a subject comprising contacting a tissue sample or cell sample with the modified aptamer handle conjugate of any preceding aspect (such as, for example an aptamer-handle conjugate comprising an aptamer as set forth in SEQ ID NO: 16 or SE ID NO: 17); (b) measuring the presence, absence or quantity of the aptamer or the transferred handle; and (c) diagnosing and/or detecting the disease state of the tissue or cell based on the presence, absence or quantity of the aptamer or covalently transferred handle measured.

In one aspect, disclosed herein are methods of detecting the presence or quantifying the amount of a target protein comprising contacting the target protein with the modified aptamer-handle conjugate of any preceding aspect. For example, disclosed herein are methods of detecting the presence or quantifying the amount of a target protein comprising contacting the target protein with a modified aptamer-handle conjugate comprising an aptamer, an electrophilic leaving group, and a handle; wherein the handle comprises or can be modified with a detectable label (such as, for example, (biotin, a bioconjugation handle, a chemiluminescent marker, a fluorescent marker, a radiomarker, a dye, a quantum dot, an enzyme, an enzyme substrate, a catalyst, or a small molecule ligand). In some aspects, the method can further comprise measuring the presence, absence or quantity of the bound aptamer relative to a control or standard. Additionally, in some aspects the methods can further comprise transferring the target protein to a membrane after contacting the target protein with the aptamer-handle conjugate.

Also disclosed herein are methods of treating, inhibiting, reducing, decreasing, ameliorating, and/or preventing a coronavirus infection (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) comprising administering to a subject with a coronavirus infection the modified aptamer handle conjugate of any preceding aspect, wherein the aptamer selectively binds the coronal virus spike protein or the receptor binding domain of the coronavirus spike or nucleocapsid protein (such as, for example, the aptamer as set forth in SEQ ID NO: 9, SEQ ID NO: 10, SEQ ID NO: 11, SEQ ID NO: 12, SEQ ID NO: 13, SEQ ID NO: 14, and/or SEQ ID NO: 15).

In one aspect, disclosed herein are methods of treating, inhibiting, reducing, decreasing, ameliorating, and/or preventing a circulatory condition (such as, for example, thrombosis, thromboembolism, Paget-Schroetter disease, fibrosis, stroke, or myocardial infarction) in a subject comprising administering to the subject the modified aptamer handle conjugate (such as, for example, a modified aptamer-handle conjugate comprising an aptamer that binds thrombin, including, but not limited to, an aptamer as set forth in SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8) of any preceding aspect.

Also disclosed herein are methods of treating, inhibiting, reducing, decreasing, ameliorating, and/or preventing a cancer and/or metastasis (such as, for example, retinoblastoma, colon cancer, lung cancer, and/or gastric cancers) in a subject comprising administering to the subject the modified aptamer handle conjugate of any preceding aspect (such as, for example an aptamer-handle conjugate comprising an aptamer as set forth in SEQ ID NO: 16 or SE ID NO: 17).

In one aspect, disclosed herein are methods of making aptamer-handle conjugate comprising a) constructing a nucleic acid sequence via solid-phase oligonucleotide synthesis; b) replacing one or more thymidines with triisopropylsilyl (TIPS) protected 5-ethynyl-2′-deoxyuridine (EdU) or other alkyne-bearing phosphoramidites such as 5-(octa-1,7-diynyl)-2′-deoxyuridine (OdU); and c) conjugating an electrophile (such as, for example, N-acyl sulfonamide cleavable electrophile or tosyl cleavable electrophile) and handle to the aptamer via copper catalyzed [3+2] cycloaddition or amine-NHS ester coupling or any other bioconjugation, including, but not limited to disulfide exchange, nucleophilic substitution of halo acetamides with thiols, thiol-maleimide conjugation, NHS-ester conjugation to amines, sulfonyl chloride conjugations to amines, isocyanate conjugation to amines, isothiocyanate conjugation to amines, aldehyde conjugation to amines through reductive amination, amine conjugation through transition-metal catalyzed reductive alkylation, thiolene reaction, copper-catalyzed azide-alkyne [3+2] cycloaddition, strain-promoted azide-alkyne [3+2] cycloaddition, aldehyde-aniline conjugation through a Mannich reaction, palladium-catalyzed allylation, glyoxylate-amine conjugation, native chemical ligation, Suzuki coupling, Sonagashira coupling, Heck coupling, inverse electro demand Diels-Alder reaction, tetrazine ligation, oxime ligation, Staudinger ligation, traceless Staudinger ligation, photoclick 1,3-dipolar cycloadditions, [4+1] isonitrile cycloadditions, 2-acylboronic acid condensation, hydrazone formation, and olefin metathesis. In some aspects, the electrophile to the aptamer occurs via the aryl sulfonamide of the electrophile.

In some aspects, the conjugation of the electrophile to the aptamer occurs via the amide motif of the electrophile. In one aspect disclosed herein are methods of making aptamer-handle conjugate wherein the electrophile comprises an N-acyl sulfonamide; and the conjugation of the electrophile to the aptamer occurs via the non-cleavable benzamide nitrogen, any non-cleavable connection to the aryl ring of the sulfonamide group, or via the carbonyl group of the cleavable amide. In some aspects, disclosed herein are methods of making aptamer-handle conjugate wherein the electrophile comprises a tosyl electrophile; and wherein the tosyl electrophile is connected to the aptamer via the non-cleavable benzamide nitrogen, via any non-cleavable connection to the aryl ring of the tosylate group, or via the alpha-CH₂ carbon of the cleavable tosylate moiety.

Also disclosed herein are method of delivering a biotin, a bioconjugation handle, a fluorescent marker, radiomarker, dye, quantum dot, enzyme, enzyme substrate, catalyst, small molecule ligand, PROTAC® (E3 ligase ligand), or LYTAC® (cation-independent mannose-6-phosphate receptor (CI-M6PR) ligand) to a target cell comprising conjugating a labeled electrophile to an aptamer creating an aptamer-handle conjugate; and contacting the target cell with said aptamer-handle conjugate.

In one aspect, disclosed herein are methods of labeling one or more protein targets on or in a cell (including but not limited to intracellular protein targets and protein targets on the cell membrane) as well as free proteins (including but not limited to serum proteins and other extracellular proteins), proteins on the surface of exosomes and/or proteins on a microbial surface (including, but not limited to viral cell surface proteins) comprising conjugating a labeled electrophile to an aptamer creating an aptamer-handle conjugate; and contacting the cell (or other surface) with the aptamer electrophile conjugate.

A method is also provided for identifying aptamers suitable as ligands for a target from a candidate mixture of modified aptamers, the method comprising:
a) preparing a candidate mixture of modified aptamers, wherein each modified aptamer may independently be selected from a modified aptamer of Formula II-a

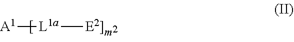

wherein:
A¹ comprises an aptamer;
L^{1A} is a linker comprising at least one cleavable moiety;
E² is an electrophilic moiety; and
m² is at least 1;
b) contacting the candidate mixture of modified aptamers with the target, wherein modified aptamers capable of forming a covalent bond with the target provide a subpopulation of covalently bound aptamers;
c) partitioning the subpopulation of covalently bound aptamers from a remainder of the candidate mixture of modified aptamers;
d) contacting the subpopulation of covalently bound aptamers with a reagent capable of reacting with the at least one cleavable moiety, wherein the at least one cleavable moiety is cleaved to provide a subpopulation of modified aptamers; and
e) amplifying the subpopulation of modified aptamers such that aptamers suitable as ligands for the target may be identified.

In some embodiments, steps a) to e) are repeated on the subpopulation of modified aptamers until an enriched mixture of modified aptamers suitable as ligands for the target is obtained.

A method is also provided for identifying aptamers suitable as ligands for a target from a candidate mixture of modified aptamers, the method comprising:
a) preparing a candidate mixture of modified aptamers, wherein each modified aptamer may independently be selected from a modified aptamer of Formula II

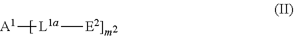

wherein:
A¹ comprises an aptamer;
L¹ is a bond or a linker;
E² is an electrophilic moiety; and
m² is at least 1;
b) contacting the candidate mixture of modified aptamers with the target, wherein modified aptamers capable of forming a covalent bond with the target provide a subpopulation of covalently bound aptamers;
c) partitioning the subpopulation of covalently bound aptamers from a remainder of the candidate mixture of modified aptamers;
d) contacting the subpopulation of covalently bound aptamers with a reagent capable of cleaving the target bound to the aptamer; and
e) amplifying the subpopulation of modified aptamers such that aptamers suitable as ligands for the target may be identified.

In some embodiments, steps a) to e) are repeated on the subpopulation of modified aptamers until an enriched mixture of modified aptamers suitable as ligands for the target is obtained. In some embodiments, the target is a protein, and the reagent is capable of degrading the protein.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a general scheme of handle transferring nucleic acid ligands. An aptamer directs its conjugated functional label and cleavable electrophile to a target protein. A nucleophilic residue quickly reacts with the aptamer-conjugated electrophile resulting in an irreversible transfer of the handle to the protein.

FIG. 2 shows chemical synthesis of 5-ethynyl-2′-deoxyuridine phosphoramidite (EdU, S1).

FIG. 3 shows chemical synthesis of tosyl electrophile 1 capable of transferring biotin to a proximal nucleophilic residue.

FIG. 4 shows a chemical synthesis of TIPS-EdU phosphoramidite S2.

FIG. 5 shows individual aptamers with specific thymidines modified with electrophile A) 1 or B) 2 were incubated with thrombin for 4 h and 1 h, respectively, and biotinylation efficiency was analyzed by western blot. The T13 and T3-modified aptamers showed most efficient label transfer to the protein, respectively.

FIG. 6 shows chemical synthesis of N-acyl sulfonamide electrophile 2.

FIG. 7 shows a biotinylation comparison of aptamers modified with electrophiles 1 and 2 at the most efficient position.

FIG. 8 shows a binding affinity comparison of three thrombin aptamers (modified and unmodified) using a fluorescence polarization assay.

FIG. 9A shows the crystal structure of TBA (blue) complexed with thrombin (pink). A zoomed-in image shows that T3 is proximal to labeled lysines (green) and distant from non-labeled ones (yellow), based on mass spec sequencing. PDB 4DII.

FIG. 9B shows a diagram highlighting labeling yields of biotinylated lysines (green) and unmodified lysines (red) as determined by mass spec. Peptides not covered by mass spec are shown in grey.

FIG. 10A shows a western blot of dose-dependent thrombin biotinylation by TBA(3)-2.

FIG. 10B shows a comparison by western blot of thrombin biotinylation between TBA(3)-2 and unconjugated 2 at increasing concentrations.

FIG. 11A shows a selectivity experiment in the presence of excess BSA.

FIG. 11B shows a selectivity experiment in human plasma.

FIG. 11C shows thrombin biotinylation time-course experiment. Error bars represent standard deviations from the average of at least two independent experiments.

FIGS. 12A, 12B, 12C, 12D, and 12E show analysis of the degree of biotinylation of thrombin by 12A) 1 μM, 12B) 0.5 μM, 12C) 0.25 μM, 12D) 0.12 μM, or 12E) 0.06 μM TBA(3)-2 after a 1 h incubation period using ion traces of whole protein LC-MS. Ion traces were gated such that the peak for each biotinylated thrombin can be individually integrated. Percentages represent the areas under each individual peak divided by the total area of all four peaks.

FIGS. 13A, 13B, and 13C show fluorescent handle transfer analyzed on a denaturing gel. FIG. 14A shows DEACM N-acylsulfonamide electrophile structure. FIG. 14B shows selective handle transfer to thrombin in the presence of excess BSA using the conjugate compared to non-selective labeling using the unconjugated electrophile. FIG. 14C shows selective handle transfer to thrombin in human plasma.

FIG. 14 shows the chemical synthesis of the DEACM-transferring electrophile 3.

FIG. 15A shows aptamer-protein crosslinking using the inverted electrophile.

FIG. 15B shows concentration-dependent aptamer-protein crosslinking.

FIG. 15C shows a selectivity experiment in human plasma.

FIG. 15D shows a time-course aptamer crosslinking experiment.

FIG. 15E shows fibrinogen clotting assay using 500 nM of thrombin. Error bars represent standard deviations from the average of at least two independent experiments.

FIG. 16 shows the chemical synthesis of the crosslinking electrophile 4.

FIG. 17 shows dose-dependent crosslinking of TBA(3)-4 with thrombin. Analyzed via SDS-PAGE and visualized with a silver stain.

FIG. 18 shows a time-course crosslinking experiment using TBA(3)-4. Analyzed via SDS-PAGE and visualized with a silver stain.

FIG. 19 shows a fibrinogen clotting assay.

FIGS. 20A, 20B, and 20C show aptamer stability in human plasma. FIG. 20A shows stability of unmodified TBA in human plasma. Analyzed via SDS-PAGE and visualized via fluorescence imaging. FIG. 20B shows stability of TBA(3)-4-thrombin conjugate in human plasma. Analyzed via SDS-PAGE and visualized via fluorescence imaging. FIG. 20C shows plotted band integrations results.

FIG. 21A shows a comparison of three covalent aptamer-mediated approaches for thrombin detection.

FIG. 21B shows a zoomed-in representation of the data (0-15 nM) from FIG. 21A. Error bars represent standard deviations from the average of at least two independent experiments.

FIGS. 22A, 22B, and 22C show thrombin detection limit experiments as described in FIG. 21. FIG. 22A shows detection using TBA(3)-2 analyzed via a western blot. FIG. 22B shows detection using TBA(3)-3 analyzed via SDS-PAGE and fluorescence imaging.

FIG. 22C shows detection using 32P-TBA(3)-4 analyzed via radiography.

FIG. 23A shows chemical structures of warheads with cleavable bonds indicated.

FIG. 23B shows warhead position dependent biotinylation of thrombin. Aptamer (1 μM) and thrombin (300 nM) were incubated in PBS (1 h, 37° C.)

FIG. 24 shows the crystal structure (PDB 6RTI) of PBA (blue) complexed with PTK7 (pink). The aptamer is proximal to lysines (green) and one cysteine (orange) at its protein binding interface. Highlighted lysines/cysteines are <20 Å away from the aptamer.

FIG. 25 shows the structure of an alkyne-modified G amidite.

FIG. 26 shows the chemical structure of a N-tetrazine-delivering warhead.

FIG. 27 shows a Western blot analysis of PTK7 biotinylation on cell surfaces. P7BA-2 was incubated with HEK293T cells transiently expressing PTK7 for 1 h at 37° C. Only PTK+ cells yielded a band in response to increasing concentrations of aptamer.

FIG. 28A shows an IgG1 aptamer delivering a tetrazine to an antibody followed by conjugation to TCO-modified doxorubicin (star).

FIG. 28B shows the TCO-modified doxorubicin 7 includes a lysozyme sensitive dipeptide (Val-Cit) conjugated through a p-aminobenzyl carbamate that is cleaved upon lysosomal trafficking, matching the FDA-approved antibody-drug conjugate ADCETRIS®.

FIG. 29A shows a general scheme of electrophile-activating nucleic acid ligands. An aptamer directs its conjugated organocatalyst to a target protein. An acyl substrate is activated by the catalyst only in the vicinity of nucleophiles (such as Lys) on the target protein. This leads to a quick, irreversible acylation of the protein.

FIG. 29B shows structures of clickable organocatalysts 8 and 9

FIG. 29C shows structures of corresponding substrates 10 and 11.

FIG. 30 shows structures of fluorophores 13-16.

FIG. 31A shows the structure of a DNP hapten-transferring electrophile.

FIG. 31B shows a general scheme of targeted immune response elicitation through handle transfer.

FIGS. 32A and 32B show structure and function of nucleic acid-based 32A) LYTACS and 32B) PROTACS.

FIG. 33 shows the chemical synthesis of OdU phosphoramidite S3

FIG. 34A shows the structure of biotin-tethered N-acyl sulfonamide cleavable electrophile 2 that is ‘clicked on’ to RBD aptamers at indicated positions.

FIG. 34B shows RBD (250 nM) biotinylation by 100 nM RBA(n)-2 in the presence of BSA (250 nM).

FIG. 35A shows the dose response biotinylation of c-MET with aptamers modified with 2 at positions 23 and 29 in the SL1 sequence.

FIG. 35B shows the time-course c-MET biotinylation in the presence of excess BSA.

FIG. 36A shows aptamers with conjugated electrophile 2 at specified thymidines when incubated with PTK7 for 1 h and biotinylation efficiency was analyzed by western blot.

FIG. 36B shows selective labeling of PTK7 in the presence of excess BSA, visualized by western blot.

FIG. 37A shows a dose-dependent biotinylation of PTK7 (100 nM). Concentrations of sgc8c(27)-2 used were 0, 0.06, 0.12, 0.25, 0.5, and 1 μM for 1 hour. Labeling analyzed by simplified western blot with SA-HRP. Total PTK7 visualized with silver stain.

FIG. 37B shows the time course of PTK7 biotinylation. Data represent averages and error bars are standard deviations from three independent experiments.

FIG. 38 shows aptamer-mediated labeling and streptavidin pulldown of lysates from HEK293T cells expressing PTK7 and PTK7-CFP.

FIG. 38A shows a plasmid map of the assembled pcDNA3-PTK7-VSV-CFP construct.

FIG. 39B shows HEK293T cells treated with 250 nM of sgc8c(27)-2 and sgc8c(27)-biotin were lysed and pulled-down protein was analyzed by western blot.

FIG. 39C shows HEK293T cells with and without pcDNA3-PTK7-VSV-CFP transfection were treated with sgc8c(27)-2 for 4 or 0 hours and analyzed for biotinylated PTK7 via western blot blot.

FIG. 39D shows live cell fluorescent imaging of COS7 cells treated with 250 nM of sgc8c(27)-2 and sgc8c(27)-biotin and subsequently with 1 μM of unmodified sgc8c. Biotinylated proteins were conjugated to neutravidin-tetramethyl rhodamine (0.1 mg/mL) to visualize covalent PTK7 biotinylation.

FIG. 39E shows silver stain of streptavidin pull-down of HEK293T cell lysates (expressing PTK7-CFP) treated with 0.5 μM of sgc8c(27)-2 and sgc8c(27)-biotin for 1 hour.

FIG. 40A shows aptamer-mediated PTK7 cell surface biotin delivery with increasing concentrations of sgc8c(27)-2. The concentrations used were 0, 0.03, 0.06, 0.12, 0.25, 0.50 mM.

FIG. 40B shows labeled PTK7-CFP bands were integrated and normalized to total PTK7-CFP.

FIG. 40C shows a time-course PTK7-CFP labeling experiment in HEK293T cells with 250 nM of sgc8c(27)-2. Cells were labeled for 240, 120, 60, 30, 15, 5 and 0 minutes.

FIG. 40D shows labeled PTK7-CFP bands were integrated and normalized to total PTK7-CFP. Error bars are standard deviations from three independent experiments.

FIG. 41 shows labeling of endogenous PTK7 on Jurkat and Nalm-6 leukemia cell surfaces via flow cytometry. One million cells were labeled with 500 nM of sgc8c(27)-2 for 1 hour at 37° C. Cells were washed with DPBS and stained with neutravidin-tetramethyl rhodamine (TMR) for 10 minutes. Cells were analyzed for TMR fluorescence via flow cytometry and 10,000 events were recorded.

FIG. 42A shows the mechanism of aptamer-catalyzed labeling.

FIG. 42B shows chemical structures of the synthesized oxime catalyst 5 and the biotin-delivering acyl donor PyOx.

FIG. 42C shows sgc8c(27)-PyOx-mediated (500 nM) biotinylation of PTK7 (100 nM) in the presence of 4-nitro-N-(4-nitrobenzyl)benzenesulfonamide (100 uM).

FIG. 42D shows electrophile-activating transfer of biotin to thrombin (10 uM) via PyOx-modified TBA (30 uM) and 6 (150 uM).

FIG. 43A shows a covalent SELEX cycle. The electrophile-modified aptamer library is incubated with the target protein and nucleic acid molecules that display electrophiles proximal to a lysine will crosslink. The aptamer-protein conjugates are then partitioned from the rest of the library via capture on a nitrocellulose membrane or another portioning step. After electrophile cleavage, aptamers are amplified via PCR, antisense strands are digested by exonucleases, and ssDNA is conjugated to electrophilic warheads. The process is repeated until sequence convergence is observed. The most abundant aptamers are characterized.

FIG. 43B shows a chemical structure of the reductively cleavable, inverted crosslinking N-acyl sulfonamide electrophilic warhead 9, enabling aptamer release from the crosslinked protein.

FIG. 44A shows crosslinking and subsequent cleavage of pr-TBA(3)-9 to thrombin after reduction of the disulfide bond with BME.

FIG. 44B shows crosslinking of pr-TBA(3)-9 to thrombin in the presence of an excess of random oligo.

FIG. 45A shows amplification efficiency comparison of an alkyne-modified or native aptamer library.

FIG. 45B shows amplification efficiency comparison of an alkyne-modified aptamer via qPCR.

FIG. 46 shows analysis of lambda exonuclease digestion of antisense strand after PCR amplification of either alkyne-modified or native aptamer. Analyzed on a native gel and visualized with SYBR Gold.

FIG. 47 shows amplification of Lys crosslinked aptamer. Pr-TBA-4 crosslinked to Lys was PCR amplified and compared to unmodified pr-TBA-4 by analyzing amplicons on a native gel and visualizing with SYBR Gold.

FIG. 48 shows qPCR amplification of enriched aptamers. A non-crosslinking random library at 2 μM was doped with the indicated concentration of pr-TBA-4. It was then incubated with thrombin, which was then immobilized. Crosslinking sequences were partitioned from non-crosslinking ones, isolated, and detected via qPCR. As expected, negative control libraries lacking pr-TBA-4 did not amplify (not shown). Errors represent standard deviations of two technical replicates.

FIGS. 49A, 49B, and 49C show generation of randomly amino-modified oligonucleotide library. FIG. 49A shows a chemical structure of AA-UTP. FIG. 49B shows PCR of ssDNA amplification analyzed on an agarose gel visualized with ethidium bromide.

FIG. 49C shows that transcripts generated in the presence of varying amounts of AA-UTP analyzed on an agarose gel visualized with ethidium bromide.

FIGS. 50A and 50B show determination of number of amines per randomized region. FIG. 50A shows denaturing PAGE analysis of libraries conjugated fluorescein-NHS.

FIG. 50B shows a standard curve of fluorescence intensity created using fluorescein-NHS. This curve was used to calculate fluorescein concentrations in a solution of known oligonucleotide concentration.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” or “drug” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular aptamer is disclosed and discussed and a number of modifications that can be made to a number of molecules including the aptamer are discussed, specifically contemplated is each and every combination and permutation of aptamer and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

A recent approach to modifying proteins has been the modification of small molecule inhibitors and ligands with electrophilic “warheads”. In contrast to traditional, reversible protein-ligand interactions, a covalent ligand is designed such that the reversible association is followed by proximity-driven, irreversible crosslinking between a strategically placed electrophile on the ligand and a nucleophilic residue on the target protein. Small molecule covalent ligands offer several advantages over their non-covalent counterparts, including the ability to outcompete endogenous ligands with similar affinities, dosage at lower concentration, discrimination between closely related protein targets based on the presence of a specific nucleophilic amino acid, and a duration of action that match the protein turnover rate. The disclosed aptamers function in a novel way by covalently transferring a handle onto the protein from an electrophilic “warhead” designed for selective bond formation with a proximal nucleophilic residue on the target, such as a Cys, Lys, or Tyr. The warhead was designed to generate a covalent link between the targeted protein and a synthetic functional group (e.g., biotin, fluorophore, bioconjugation handle, etc), simultaneously releasing it from the aptamer. This approach enables new aptamer applications, including:

### Sensitive Protein Detection.

Native protein labeling and aptamer conjugation enables target detection via several modalities. A biotin handle provides luminescence-based detection where primary and secondary antibodies in western blots are replaced with a streptavidin-horseradish peroxidase conjugate. Fluorescent handles enable multiplexed protein detection in simple experimental workflows. Crosslinking aptamers provide ultrasensitive detection through ³²P-radiography and qPCR.

### Protein Bioconjugation.

Chemical modification of proteins is essential for many biologics (e.g., antibody-drug conjugates and PEGylated proteins) as well as studies of protein function, dynamics, folding, localization, interaction, etc. Virtually all protein- and site-specific bioconjugation approaches require various levels of genetic manipulation, not allowing for the selective modification of native proteins. Statistical labeling of lysines (e.g., using NHS-esters) leads to heterogenous mixtures, in particular in native environments (e.g., lysates), and batch-to-batch variability often leads to inconsistent results. The disclosed approach addresses these limitations through selective and direct covalent handle transfer.

### Targeted Protein Degradation.

Targeted protein degraders (PROTACs and LYTACs) have promising research and therapeutic applications. These heterobifunctional molecules diverge from traditional inhibitors by degrading their protein targets, thereby abolishing even non-enzymatic functions. However, many protein classes do not contain small molecule binding pockets, such as most transcription factors, and thus are inaccessible to these strategies. The disclosed aptamers, however, have no such limitation and enable protein degradation of a significantly broadened range of targets. Moreover, covalent labeling is fast and subsequent nuclease-mediated aptamer degradation will have no effect on protein degradation.

### Immune Response Redirection.

Recruitment of endogenous antibodies to cell surface receptors via small molecules in a promising strategy to elicit a targeted immune response. Unfortunately, small molecules fail to emulate antibody specificities and binding kinetics, resulting in reduced immune stimulation. The disclosed aptamers, however, have specificities that rival those of antibodies and are not limited to small molecule binding sites. Furthermore, the covalent labeling results in a permanent target association, which is important because an immune response is contingent on average ligand-receptor lifetimes >6 h. These residence times are unachievable with current aptamer technologies and thus are uniquely enabled by the approach disclosed herein.

In order to expand the scope of covalent labeling beyond small molecule ligands and their corresponding targets, we are utilizing nucleic acid aptamers, an important class of macromolecules capable of recognizing proteins with high affinity and specificity. In contrast to small molecule ligands, aptamers can be selected for virtually any protein target, including toxic and nonimmunogenic proteins, using a generalized SELEX approach and have found utility for a broad range of targets, including thrombin, RBD, c-MET, and PTK7.

In one aspect, disclosed herein are modified aptamer-handle conjugates comprising an aptamer (including, but not limited to the aptamers as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEDQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and/or SEQ ID NO: 20), an electrophilic leaving group (such as, for example, N-acyl sulfonamide cleavable electrophile or tosyl cleavable electrophile) and a handle. Also disclosed herein are modified aptamers (including, but not limited to the aptamers as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and/or SEQ ID NO: 20) comprising an electrophilic leaving group (such as, for example, N-acyl sulfonamide cleavable electrophile or tosyl cleavable electrophile) for crosslinking a target.

Aptamers are short, single-stranded nucleic acid molecules that are generated through powerful selection strategies and bind targets with exquisite affinity and specificity. They are versatile nucleic acid tools with numerous applications as biological probes, as well as therapeutic, diagnostic, and drug delivery agents.

Aptamers' broad scope in protein binding complements the functionality provided by transferable synthetic motifs in a powerful fashion. Aptamers interact with their protein targets over a large surface area; therefore, chemical modification of a single nucleotide is unlikely to abolish their binding affinity. Whereas small molecule inhibitors and ligands are often unamenable to such change. Additionally, oligonucleotides are generated via automated chemical synthesis and thus are easily (commercially) accessible.

In some embodiments, the disclosed aptamer has a sequence selected from the sequences shown in Table 1. In some embodiments, the disclosed aptamer has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequences shown in Table 1.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-JH-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

### 1. Modified Aptamers

In one aspect, a modified aptamer-handle conjugate is provided of Formula I

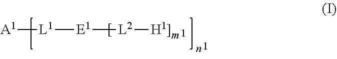

wherein:
A¹ comprises an aptamer;
L¹ and L² are independently selected from a bond or a linker;
E¹ is a cleavable electrophilic moiety;
H¹ is a handle;
m¹ is at least 1; and
n¹ is at least 1.

As used herein, a “cleavable electrophilic moiety” refers to a chemical moiety which is capable of reaction with a nucleophilic functional group or moiety of the target and which, upon reaction with the nucleophilic functional group or moiety of the target, leads to the breaking of one or more chemical bonds in the cleavable electrophilic moiety. In some embodiments, reaction of the nucleophilic functional group or moiety of the target leads to substitution of the target with a moiety which includes the H1 group. In other embodiments, reaction of the nucleophilic functional group or moiety of the target leads to substitution of the target with a moiety which includes the A¹ group.

In some embodiments of Formula I, E¹ may be selected from a tosylate moiety, an acyl imidazole moiety, an electrophilic phenyl benzoate moiety, a N- or O-sulfonyl pyridine moiety, or an N-acyl sulfonamide moiety.

In some embodiments of Formula I, E¹ is selected from

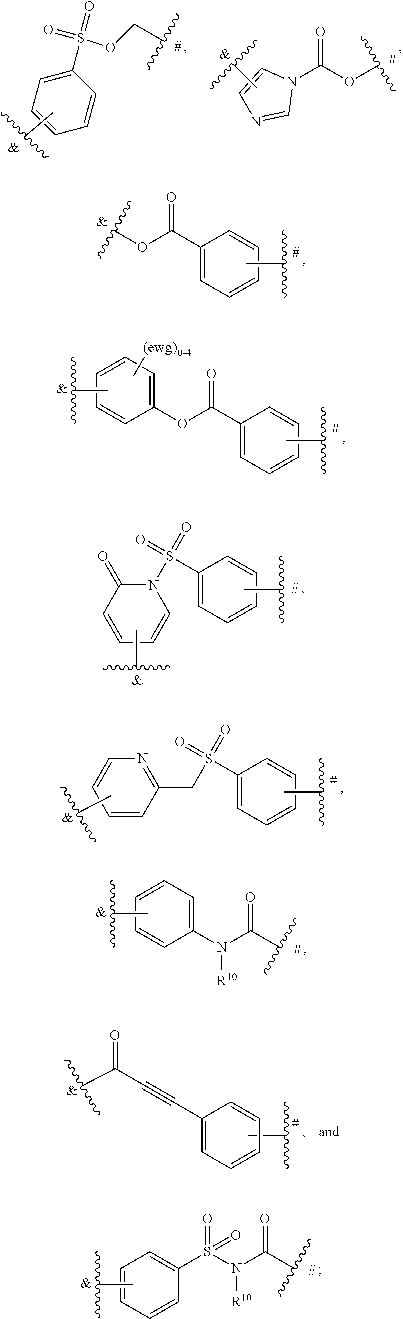

wherein:

one of & and # is the point of attachment to L¹ and the other of & and # is the point of attachment to L²;

ewg is independently at each occurrence an electron withdrawing group (for example, a halogen, nitro, or haloalkyl group); and

R¹⁰ is selected from hydrogen, alkyl, cycloalkyl, heterocyclyl, aryl, or heteroaryl, wherein R¹⁰ may be optionally substituted as allowed by valency; and wherein each E¹ may be optionally substituted with one or more substituents as allowed by valency, as described herein.

Representative examples of E¹ include, but are not limited to:

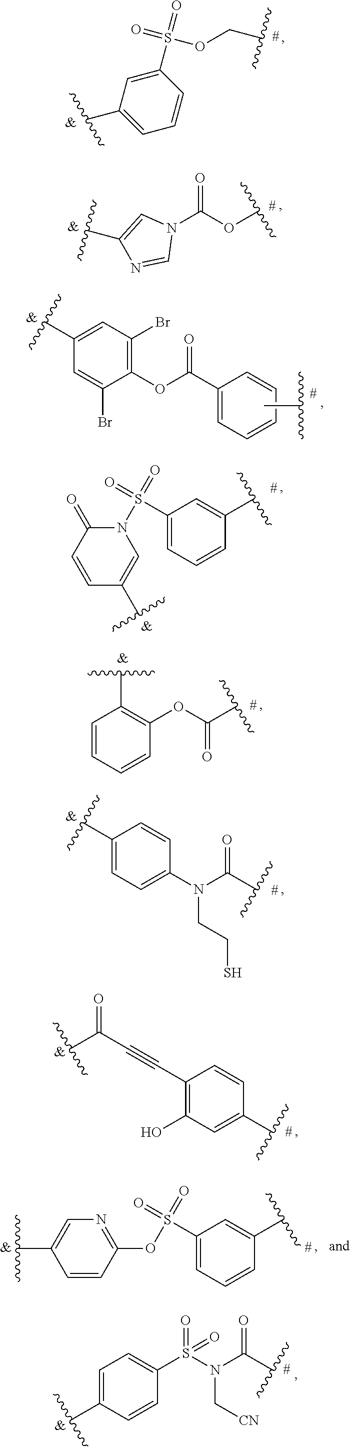

wherein one of & and # is the point of attachment to L¹ and the other of & and # is the point of attachment to L².

In some embodiments, E¹ is a moiety substituted with a group selected from:

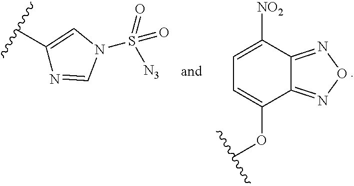 [SMILES: *c1cn(S(=O)(=O)N=[N+]=[N-])cn1 ; *Oc1ccc([N+](=O)[O-])c2nonc12]

Aptamer conjugates of Formula I comprise an H¹ group, wherein H¹ is a handle. As used herein, a handle comprises any detectable moiety including, but not limited to a labels (including biotins, bioconjugating groups, a fluorescent marker (e.g., fluorophores), radiomarkers, organocatalysts, dye, quantum dot, enzyme, enzyme substrate, and other detectable markers), metal catalyst, small molecule ligands, drugs, PROTAC® (E3 ligase ligand), or LYTAC® (cation-independent mannose-6-phosphate receptor (CI-M6PR) ligand) or small molecule inhibitors). A label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorophores) and enzymes or chemiluminescent markers that react with colorimetric substrates (e.g., horseradish peroxidase).

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™-Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18 (5)); DIDS; Dihydorhodamine 123 (DHR); Dil (DilC18 (3)); I Dinitrophenol; DiO (DiOC18 (3)); DiR; DiR (DilC18 (7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO—PRO-I; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation and other reactions. Examples of radionuclides useful in this embodiment include, but are not limited to, tritium, iodine-125, iodine-131, iodine-123, iodine-124, astatine-210, carbon-11, carbon-14, nitrogen-13, fluorine-18, and phosphorous-32. In another aspect, the radionuclide can be attached to a linking group or bound by a chelating group, which is then attached to the compound directly or by means of a linker. Examples of radionuclides include, but are not limited to, P-32, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The labeled compounds are useful as imaging agents to diagnose diseases, study protein expression, detect biomarkers or to follow the progression or treatment of such a disease or condition in a mammal (e.g., a human). The labeled compounds described herein can be conveniently used in conjunction with imaging techniques such as positron emission tomography (PET) or single photon emission computerized tomography (SPECT), flow cytometry, mass spectroscopy, and immunoassays.

Examples of small molecule handles that can be used in the aptamer-handle conjugates in, for example, the treatment of cancer include, but are not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). In some instances in handle can include moieties that block PD-1 (such as, for example, Nivolumab (BMS-936558 or MDX1106), CT-011, MK-3475), PD-L1 (such as, for example, MDX-1105 (BMS-936559), MPDL3280A, or MSBOO10718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGDO09, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, - and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep). In some embodiments, the handle comprises a bioconjugating group.

Representative examples of such groups include, but are not limited to, moieties containing a terminal alkyne, an azide, a tetrazine, a strained alkyne, a strained alkene, a phosphine, a chloroalkane, a benzylguanine, a benzylcytosine, and the like. Other suitable groups which may be used in bioconjugation are known in the art.

In another aspect, a modified aptamer is provided of Formula II

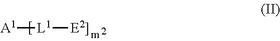

wherein:
A¹ comprises an aptamer;
L¹ is selected from a bond or a linker;
E² is an electrophilic moiety; and
m² is at least 1.

As used herein, an “electrophilic moiety” refers to a chemical moiety which is capable of reaction with a nucleophilic functional group or moiety of the target and which, upon reaction with the nucleophilic functional group or moiety of the target, leads to substitution of the target with a moiety containing the A¹ group.

Examples of electrophilic moieties E² for use in the disclosed compositions and methods include, but are not limited to: iodoacetamides, bromoacetamides, chloroacetamides, pentafluorophenylsulfonamides, 2-bromo-N-(prop-2-yn-1-yl)benzo[d]thiazole-6-carboxamides, 2-(methylsulfonyl)benzo[d]thiazole-6-carboxamides, 2-(methylsulfonyl)-4-phenyl-1,3,4-oxadiazoles, 5-methyl-1,2-benziodoxol-3 (1H)-ones, oxiranes, maleimides, aryl and alkyl propiolamides, aryl and alkyl acrylamides, aryl and alkyl vinylsulfonamides, 2-fluoro-N-(hex-5-yn-1-yl)acrylamides, 2-thioxothiazolidines, N-acyl sulfonamides, 4-methoxycyclobut-3-ene-1,2-diones, 3-methoxy-4-(phenylamino)cyclobut-3-ene-1,2-diones, 5-(prop-2-yn-1-yloxy)benzaldehydes, 4-(hydroxymethyl)-3-nitro-benzamides, 1H-1,2,3-triazole-4-carbaldehydes, 3-phenyl-2H-azirines, (Z)—N-phenylacetohydrazonoyl chlorides, benzenesulfonyl fluorides, 3H-1,2,4-triazole-3,5 (4H)-diones, benzenediazonium tetrafluoroborates, (3-phenyl-1,2-oxaziridin-2-yl)(4-piperidin-1-yl)methanones, 2,4,6-trimethyl-1-(methylcarbonylamino)pyridin-1-ium tetrafluoroborates, phosphorodichloridothioates, 3-(hydroxymethyl)naphthalen-2-ols, 4-hydroxy-3-(hydroxymethyl)benzenesulfonamides, 4-hydroxy-3-(methoxymethyl)benzenesulfonamides, and 2,2-dihydroxy-1-phenyl)ethan-1-ones.

Representative examples of E² include, but are not limited to:

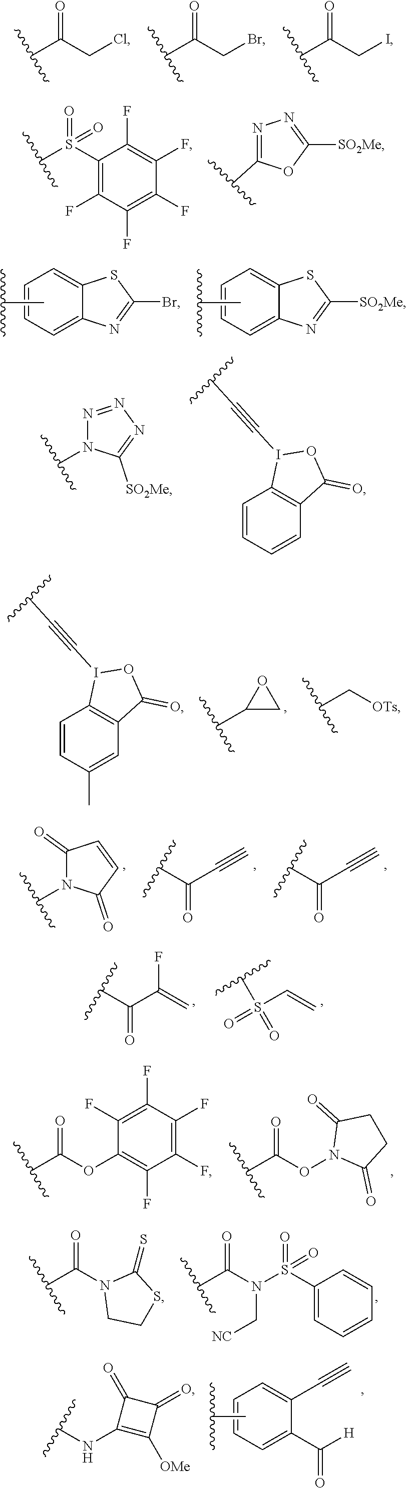

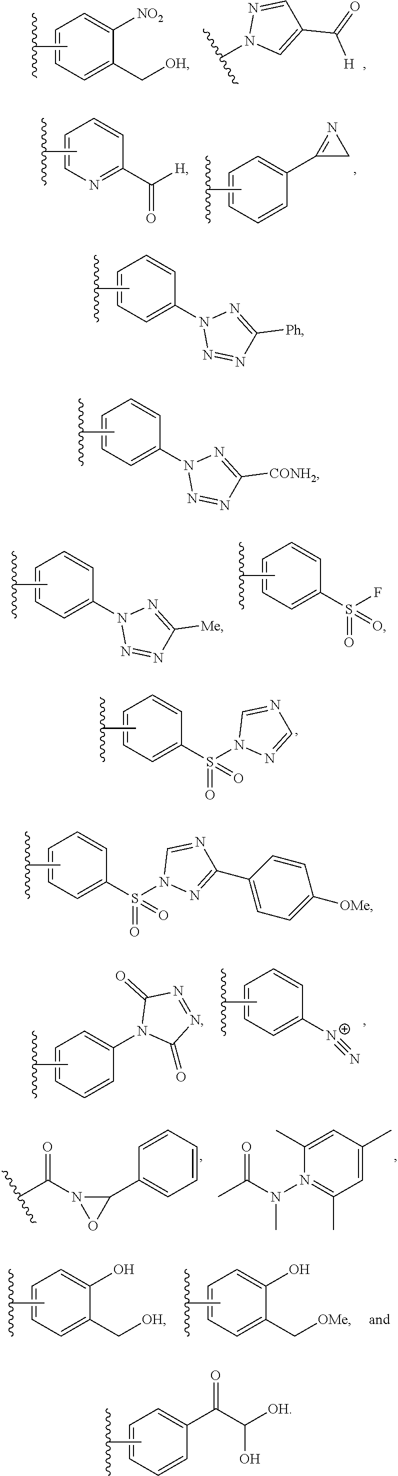

Further examples of E² include, but are not limited to:

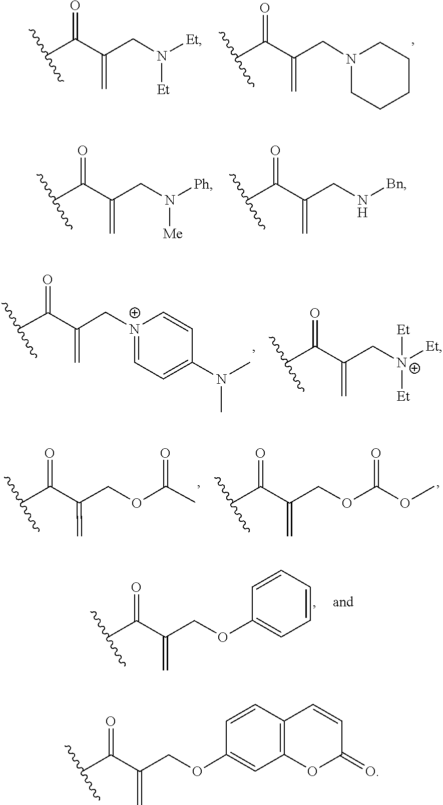

In yet another aspect, a modified aptamer is provided of Formula III

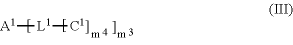

wherein:
A¹ comprises an aptamer;
L¹ is selected from a bond or a linker;
C¹ is a catalytic moiety;
m³ is at least 1; and
m⁴ is at least 1.

As used herein, a “catalytic moiety” refers to moiety which is capable of increasing the rate of chemical reaction between the target and another compound without itself undergoing any permanent chemical change.

In some embodiments, the modified aptamer of Formula III is capable of catalyzing the reaction of a compound of Formula IV

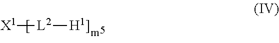

with a target, such that the target is substituted with a moiety comprising the H¹ group,
wherein X¹ is a moiety which converts into a reactive group capable of forming a covalent bond with the target upon contact with the modified aptamer of Formula III;
m⁵ is at least 1; and
all other variables are as defined herein.
   In some embodiments, the catalytic moiety C1 comprises a nucleophilic catalyst. In some embodiments, the catalytic moiety C1 comprises an aminopyridine moiety.

In some embodiments, the catalytic moiety can be

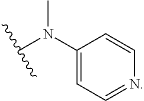 [SMILES: *N(C)c1ccncc1]

In such embodiments, X¹ may typically be a thiophenylester.

In some embodiments, the catalytic moiety can be

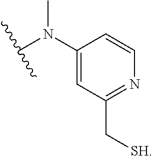 [SMILES: *N(C)c1ccnc(CS)c1]

In such embodiments, X¹ may typically be a thioester.

In some embodiments, the catalytic moiety C comprises a pyridine oxime. In some embodiments, the catalytic moiety can be

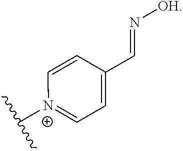 [SMILES: *[n+]1ccc(/C=N/O)cc1]

In such embodiments, X¹ may typically be

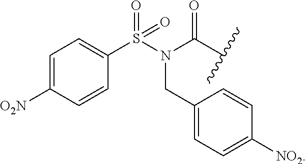 [SMILES: *C(=O)N(Cc1ccc([N+](=O)[O-])cc1)S(=O)(=O)c1ccc([N+](=O)[O-])cc1]

In some embodiments, the catalytic moiety C¹ comprises a metal catalyst. In some embodiments, the catalytic moiety comprises a rhodium catalyst. In some embodiments, the catalytic moiety comprises

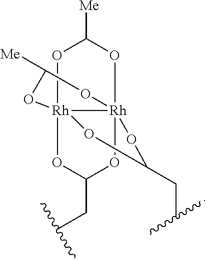

In such embodiments, X¹ may typically comprise a diazo compound. In some embodiments, X¹ may comprise

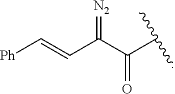

In some embodiments, the catalytic moiety C¹ comprises a photocatalyst. In some embodiments, the catalytic moiety may comprise a ruthenium photocatalyst. In some embodiments, the catalytic moiety may comprise Ru(bpy)₃. In such embodiments, X¹ may typically comprise a group selected from

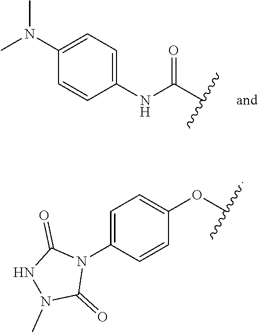

The modified aptamer-handle conjugate of Formula I and the modified aptamers of Formula II and Formula III each contain an aptamer A¹. As used herein, the term “aptamer” refers to oligonucleic acid molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits and suppresses functions of such molecule. A nucleic acid aptamer may be constituted by DNA, RNA, or a combination of both. Nucleic acid aptamers may contain numerous modifications at their nucleobase, phosphodiester backbone, and carbohydrate ring.

Nucleic acid aptamers are typically isolated from complex libraries of synthetic oligonucleotides by an iterative process of adsorption, recovery and reamplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. Specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer. Representative examples of how to make and use aptamers to bind a variety of different targets can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

In some embodiments, the disclosed aptamer A1 has a sequence selected from the sequences shown in Table 1. In some embodiments, the disclosed aptamer has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence shown in Table 1.

In some embodiments of Formula I, Formula II, or Formula III, the aptamer A¹ is bound to L¹ via a nucleotide or nucleotide analog within the aptamer A¹. The nucleotide or nucleotide analog within the aptamer A¹ may be bound to L¹ at any suitable position as allowed by valency. In some embodiments, L¹ is bound to a base moiety of the nucleotide or nucleotide analog within the aptamer A¹. In some embodiments, L¹ is bound to a sugar moiety of the nucleotide or nucleotide analog within the aptamer A¹. In some embodiments, L¹ is bound to a phosphate moiety of the nucleotide or nucleotide analog within the aptamer A¹.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers. There are a variety of molecules disclosed herein that are nucleic acid based, including for example the aptamers. The disclosed nucleic acids are made up of for example, nucleotides, nucleotide analogs, or nucleotide substitutes. Non-limiting examples of these and other molecules are discussed herein. It is understood that for example, that expressed mRNA will typically be made up of A, C, G, and U.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin9yl (A), cytosinlyl (C), guanin9yl (G), uracillyl (U), and thyminlyl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5methylcytosine (5meC), 5hydroxymethyl cytosine, xanthine, hypoxanthine, and 2aminoadenine as well as modifications at the sugar or phosphate moieties. There are many varieties of these types of molecules available in the art and available herein.

Modifications to the base moiety would include natural and synthetic modifications of A, C, G, and T/U as well as different purine or pyrimidine bases, such as uracil5yl (.psi.), hypoxanthin9yl (I), and 2aminoadenin9yl. A modified base includes but is not limited to 5methylcytosine (5meC), 5hydroxymethyl cytosine, xanthine, hypoxanthine, 2aminoadenine, 6methyl and other alkyl derivatives of adenine and guanine, 2propyl and other alkyl derivatives of adenine and guanine, 2thiouracil, 2thiothymine and 2thiocytosine, Shalouracil and cytosine, Spropynyl uracil and cytosine, 6azo uracil, cytosine and thymine, 5uracil (pseudouracil), 4thiouracil, 8halo, 8amino, 8thiol, 8thioalkyl, 8hydroxyl and other 8substituted adenines and guanines, 5halo particularly 5bromo, 5trifluoromethyl and other 5substituted uracils and cytosines, 7methylguanine and 7methyladenine, 8azaguanine and 8azaadenine, 7deazaguanine and 7deazaadenine and 3deazaguanine and 3deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289302, Crooke, S. T. and Lebleu, B. ed., CRC Press, 1993. Certain nucleotide analogs, such as 5substituted pyrimidines, 6azapyrimidines and N2, N6 and O6 substituted purines, including 2aminopropyladenine, 5propynyluracil and 5propynylcytosine. 5methylcytosine can increase the stability of duplex formation. Often time base modifications can be combined with for example a sugar modification, such as 2′-O-methoxyethyl, to achieve unique properties such as increased duplex stability.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O, S, or Nalkyl; O, S, or Nalkenyl; O, S or Nalkynyl; or OalkylOalkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙ O]ₘ CH₃, —O(CH₂)ₙ OCH₃, —O(CH₂)ₙ NH₂, —O(CH₂)ₙ CH₃, —O(CH₂)ₙ—ONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, Oalkaryl or Oaralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

Chemical modifications of a C-5 modified pyrimidine can also be combined with, singly or in any combination, 2′-position sugar modifications, modifications at exocyclic amines, and substitution of 4-thiouridine and the like. Representative C-5 modified pyrimidines include: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-phenethylcarboxyamide)-2′-deoxyuridine (PedU), 5-(N-thiophenylmethylcarboxyamide)-2′-deoxyuridine (ThdU), 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5-(N-[1-(3-trimethylamonium) propyl]carboxyamide)-2′-deoxyuridine chloride, 5-(N-naphthylmethylcarboxyamide)-2′-deoxyuridine (NapdU), 5-(N-naphthylmethylcarboxyamide)-2′O-methyluridine, 5-(N-naphthylmethylcarboxyamide)-2′-fluorouridine or 5-(N-[1-(2,3-dihydroxypropypropyl)]carboxyamide)-2′-deoxyuridine).

Nucleotides can be modified either before or after synthesis of an oligonucleotide. A sequence of nucleotides in an oligonucleotide may be interrupted by one or more non-nucleotide components. A modified oligonucleotide may be further modified after polymerization, such as, for example, by conjugation with any suitable labeling component. It is understood that nucleotide analogs need only contain a single modification, but may also contain multiple modifications within one of the moieties or between different moieties. Nucleotide substitutes are molecules having similar functional properties to nucleotides, but which do not contain a phosphate moiety, such as peptide nucleic acid (PNA).

Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 65536556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

In some embodiments, the nucleotide or nucleotide analog bound to L¹ within the aptamer A¹ may be selected from:

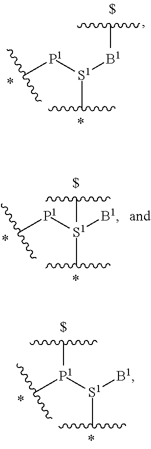

wherein:

P¹ is a phosphate moiety or derivative thereof;

S¹ is a sugar moiety or a derivative thereof or other suitable heterocycle;

B¹ is absent or selected from a base moiety or a derivative thereof,

* shows the points of attachment of the nucleotide or nucleotide analog within the aptamer A¹; and

$ shows the point of attachment to L¹.

In some embodiments, B¹ may be selected from:

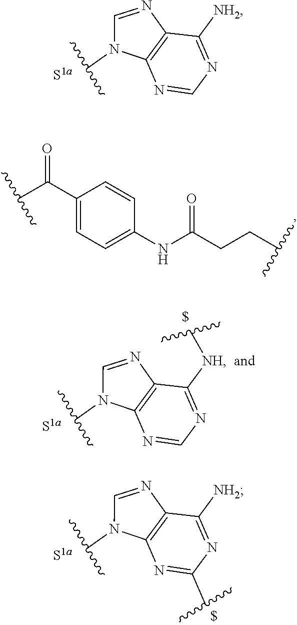

wherein s^{1a} shows the point of attachment to S¹ and $ shows the point of attachment to L¹.
In some embodiments, B¹ may be selected from:

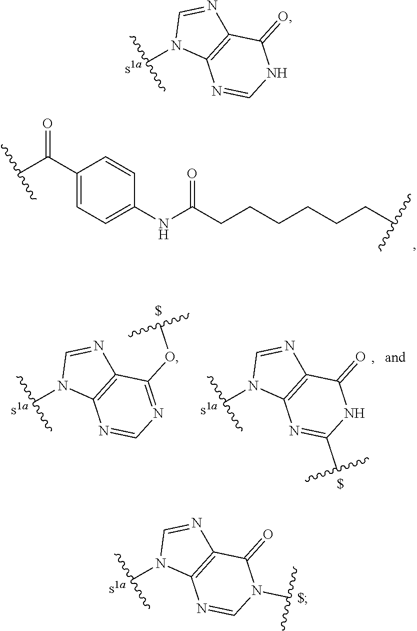

wherein all variables are as defined herein.
In some embodiments, B¹ may be selected from:

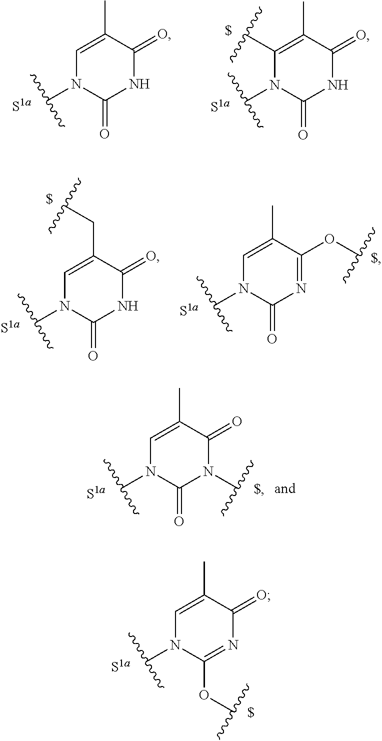

wherein all variables are as defined herein.
In some embodiments, B¹ may be selected from:

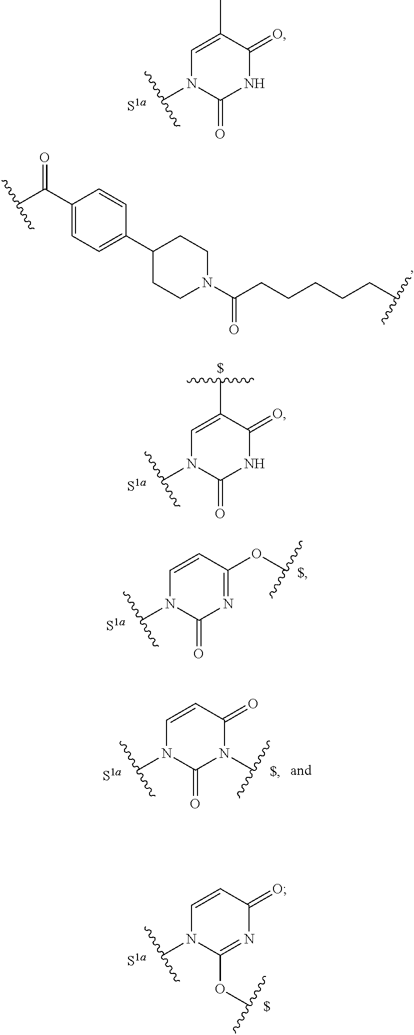

wherein all variables are as defined herein.
In some embodiments, B¹ may be selected from:

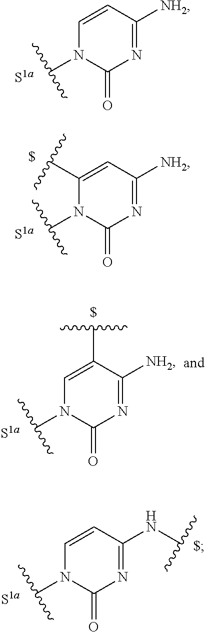 [SMILES: *n1ccc(N)nc1=O ; *c1cc(N)nc(=O)n1* ; *c1cn(*)c(=O)nc1N ; *Nc1ccn(*)c(=O)n1]

wherein all variables are as defined herein.

In some embodiments, B¹ may be absent.

In some embodiments, S¹ may be selected from:

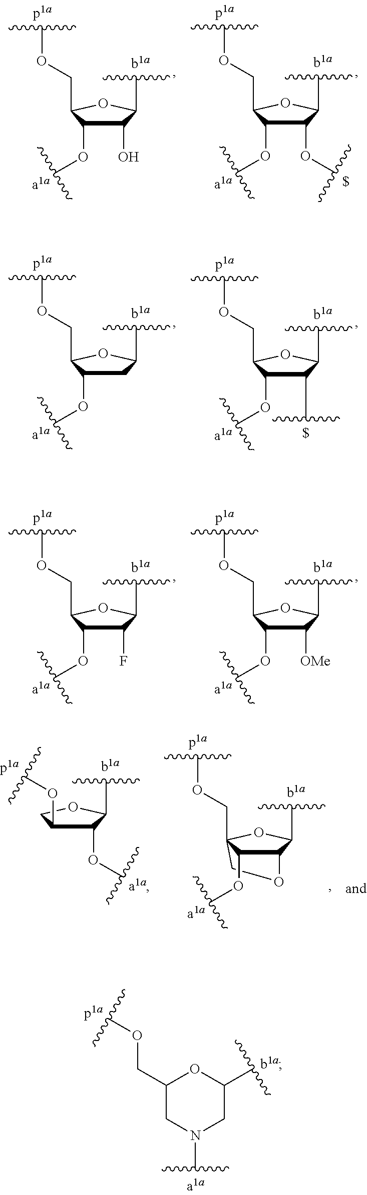

wherein:
p^{1a} shows the point of attachment to P¹;
a^{1a} shows the point of attachment of within the aptamer A¹;
b^{1a} shows the point of attachment to B¹; and
$ shows the point of attachment to L¹.

In some embodiments, P¹ may be selected from:

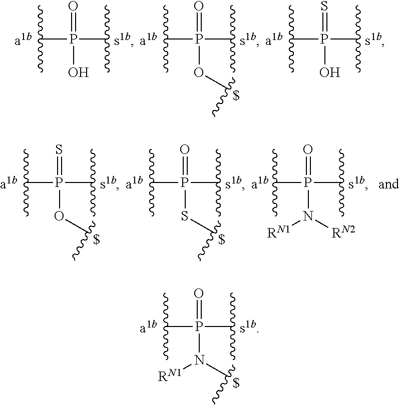

In some embodiments, the nucleotide or nucleotide analog bound to L¹ within the aptamer A¹ may comprise a peptide nucleic acid. In some embodiments, the peptide nucleic acid may comprise a moiety of the formula:

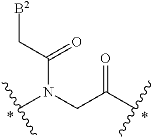

wherein:
B² is a base moiety or derivative thereof;
* shows the points of attachment of the nucleotide or nucleotide analog within the aptamer A¹

In some embodiments, the moieties L¹ and L² as found in the formulae herein may each independently comprise a linker. Linker is a chemically stable bivalent group that attaches, for L¹, A¹ to E¹(for Formula I), E² (for Formula II), or Nuⁱ(for Formula III). Linker as described herein can be used in either direction. For example in Formula I, either the left end of the linker for L¹ is linked to A¹ and the right end to E¹, or the left end is linked to E¹ and the right end to A¹.

In some embodiments, the Linker is a chain of 2 to 14, 15, 16, 17, 18, 19, or 20 or more carbon atoms, of which one or more carbons can be optionally replaced by a heteroatom such as O, N, S, or P.

In some embodiments, the chain has 2, 3, 4, 5, 6, 7, 8, 9, 10, 11, 12, 13, 14, 15, 16, 18, 19, 19, or 20 contiguous atoms. For example, the chain may include 1 or more ethylene glycol units that can be contiguous, partially contiguous or non-contiguous (for example, 2, 3, 4, 5, 6, 7, 8, 9, 10, 11, or 12 ethylene glycol units).

In some embodiments, the chain has at least 1, 2, 3, 4, 5, 6, 7, or 8 contiguous units which can be branched and which can be independently alkyl, aryl, heteroaryl, alkenyl, or alkynyl, cycloalkyl, or heterocycloalkyl substituents.

In some embodiments, the linker can include or be comprised of one or more ethylene glycol, propylene glycol, lactic and/or glycolic acid units. Block and random lactic acid-co-glycolic acid moieties, as well as ethylene glycol and propylene glycol, are known in the art and can be modified to obtain the desired half-life and hydrophilicity. In certain aspects, these units can be flanked or interspersed with other moieties, such as for example alkyl, heteroalkyl, aryl, heteroaryl, cycloalkyl, heterocycloalkyl, etc., as desired to achieve the appropriate properties.

In some embodiments, the Linker is an optionally substituted (poly)ethylene glycol having at least 1, at least 2, at least 3, at least 4, at least 5, at least 6, at least 7, at least 8, at least 9, at least 10, or more, ethylene glycol units, or optionally substituted alkyl groups interspersed with optionally substituted O, N, S, P or Si atoms.

In some embodiments, the Linker is flanked, substituted, or interspersed with an alkyl, cycloalkyl, heterocycloalkyl, aryl, or heteroaryl group.

In some embodiments, the Linker may be asymmetric or symmetric.

In some embodiments, the Linker can be a non-linear chain, and can be, or include, cycloalkyl, heterocycloalkyl, aryl, or heteroaryl cyclic moieties.

In some embodiments, the Linker is selected from L1:

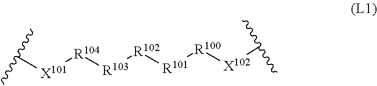

In some embodiments, the Linker is selected from the group consisting of a moiety of Formula L1, Formula L2, Formula L3, Formula L4, Formula L5, Formula L6, Formula L7, Formula L8, Formula L9, or Formula L10:

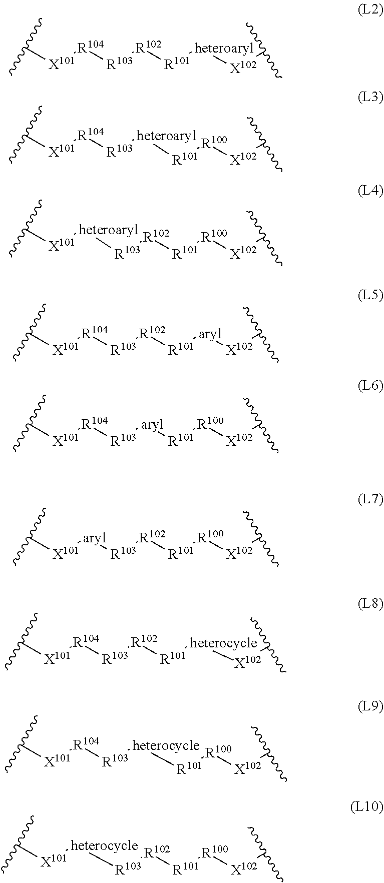

wherein:

X¹⁰¹ and X¹⁰² are independently at each occurrence selected from a bond, aryl, heteroaryl, cycloalkyl, heterocycle, NR¹³⁰, C(R¹³⁰)₂, O, C(O), and S;

R¹⁰⁰, R¹¹⁰, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)-alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;

R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;

R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and

R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl. In some embodiments, R¹⁰⁰, R¹⁰¹, R¹⁰², R¹⁰³, and R¹⁰⁴ within the Linker are selected in such manner that: no two —C(═O)— moieties are adjected to each other; no two —O— or —NH-moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of R¹⁰⁰, R¹⁰¹, R¹⁰², R¹⁰³, and R¹⁰⁴.

The following are non-limiting examples of Linkers and/or moieties which comprise Linkers in whole or in part that can be used in this invention. Based on this elaboration, those of skill in the art will understand how to use the full breadth of Linkers that will accomplish the goal of the invention.

Non-limiting examples of moieties which may comprise the Linker, either in whole or in part, include, but are not limited to: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₅—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆O—; —OCH₂—; —O(CH₂)₂—; —O(CH₂)₃—; —O(CH₂)₄—; —O(CH₂)₅—; —O(CH₂)₆—;

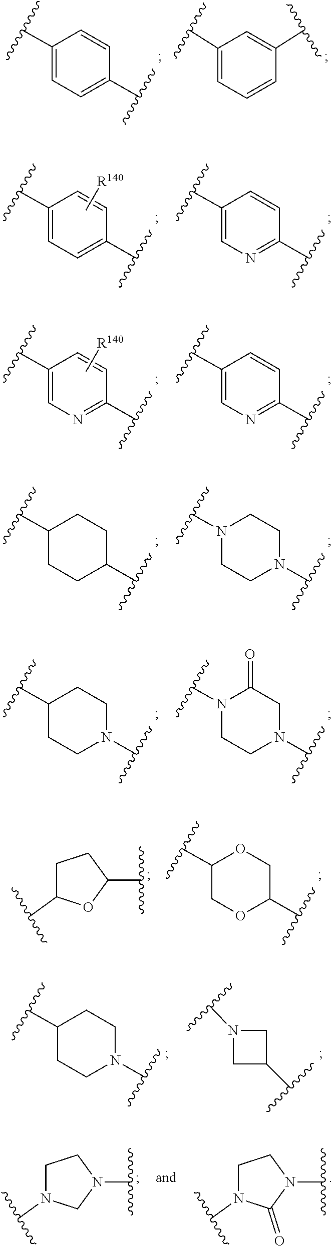

Further non-limiting examples of moieties which may comprise the Linker, either in whole or in part, include, but are not limited to:

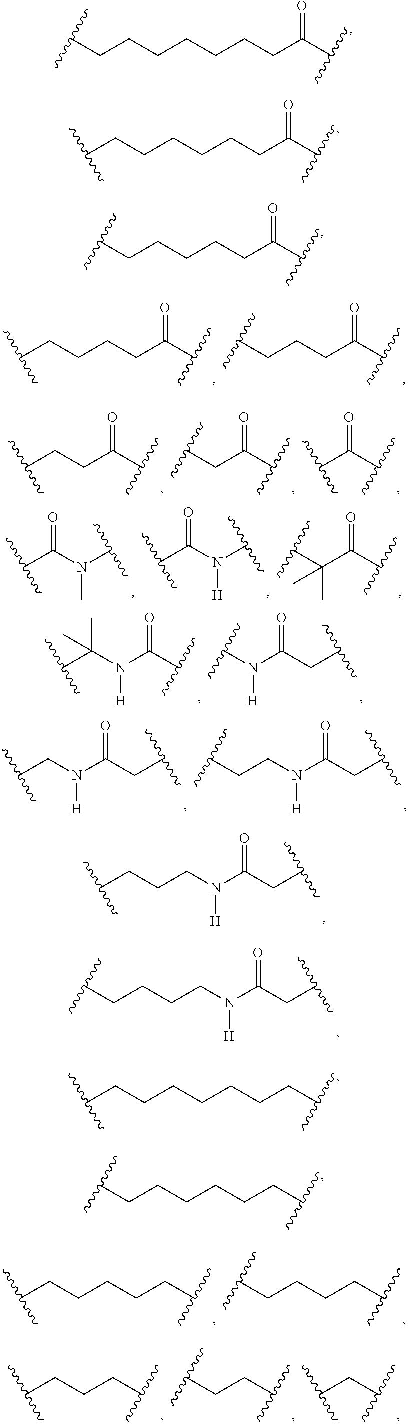

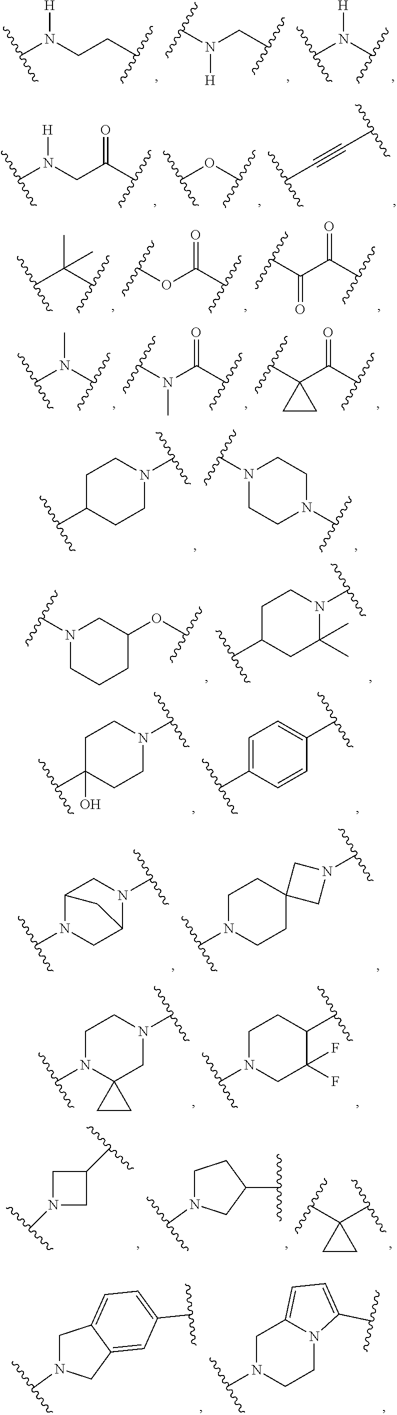

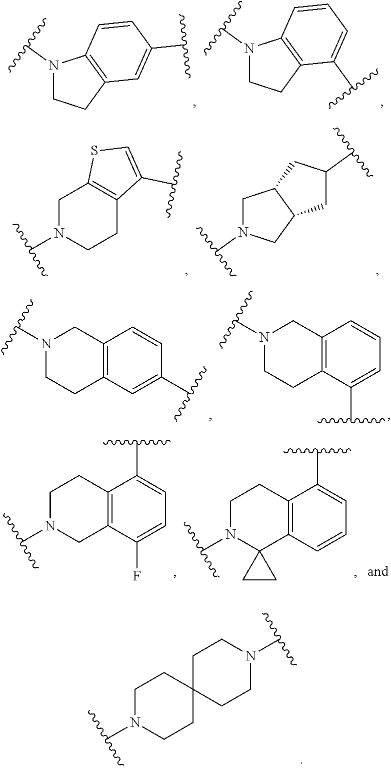

In some embodiments, the Linker may comprise, either in whole or in part, a moiety selected from:

In some embodiments, the Linker may comprise, either in whole or in part, a

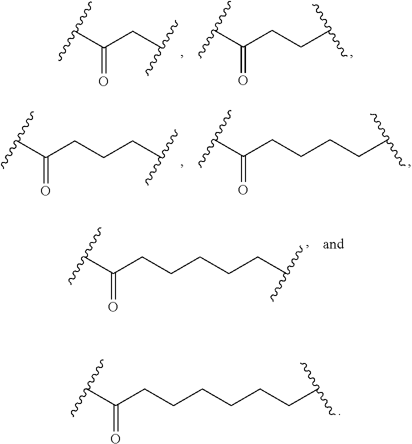 [SMILES: *CC(*)=O ; *CCC(*)=O ; *CCCC(*)=O ; *CCCCC(*)=O ; *CCCCCC(*)=O ; *CCCCCCC(*)=O]

In some embodiments, the Linker may comprise, either in whole or in part, a

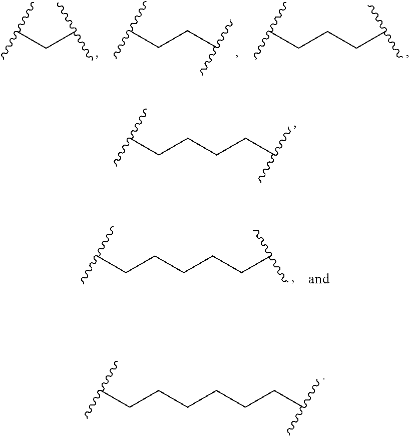

In some embodiments, the Linker may comprise, either in whole or in part, a moiety selected from:

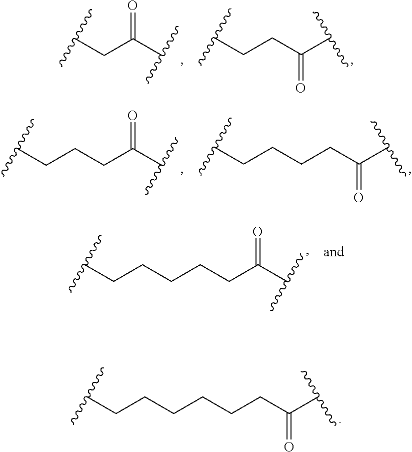 [SMILES: *CC(*)=O ; *CCC(*)=O ; *CCCC(*)=O ; *CCCCC(*)=O ; *CCCCCC(*)=O ; *CCCCCCC(*)=O]

In some embodiments, the Linker may comprise, either in whole or in part,

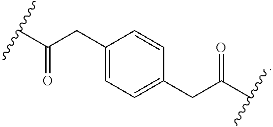 [SMILES: *C(=O)Cc1ccc(CC(*)=O)cc1]

In some embodiments, the Linker may comprise, either in whole or in part,

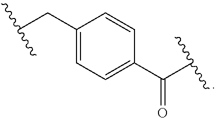 [SMILES: *Cc1ccc(C(*)=O)cc1]

In some embodiments, the Linker may comprise, either in whole or in part, a moiety selected from:

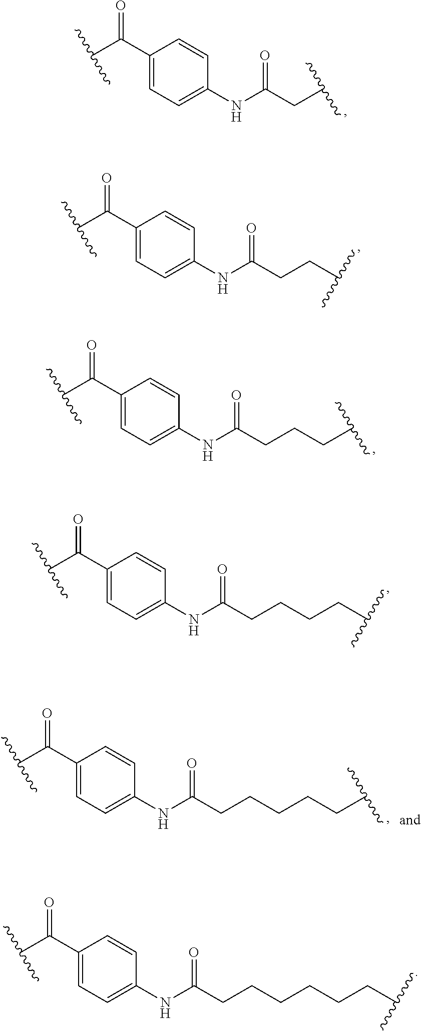 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some embodiments, the Linker may comprise, either in whole or in part, a moiety selected from:

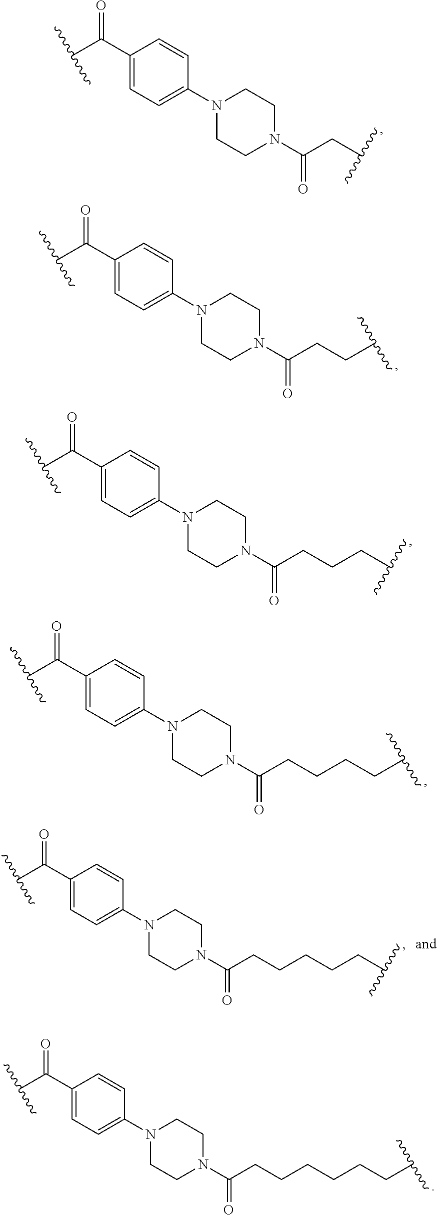 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some embodiments, the Linker may comprise, either in whole or in part, a moiety selected from:

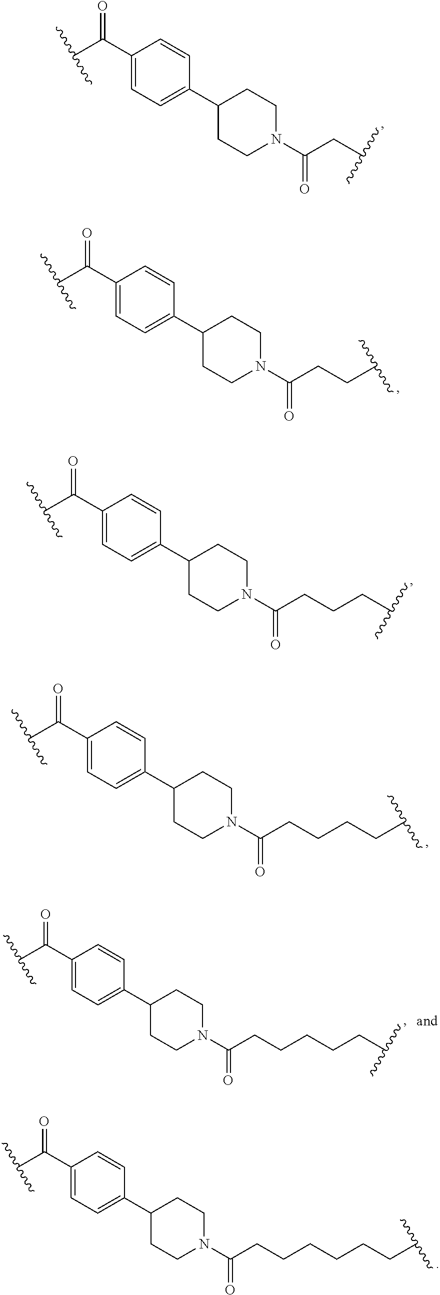 [SMILES: *CC(=O)N1CCC(c2ccc(C(*)=O)cc2)CC1 ; *CCC(=O)N1CCC(c2ccc(C(*)=O)cc2)CC1 ; *CCCC(=O)N1CCC(c2ccc(C(*)=O)cc2)CC1 ; *CCCCC(=O)N1CCC(c2ccc(C(*)=O)cc2)CC1 ; *CCCCCC(=O)N1CCC(c2ccc(C(*)=O)cc2)CC1 ; *CCCCCCC(=O)N1CCC(c2ccc(C(*)=O)cc2)CC1]

In some embodiments, the Linker may comprise, either in whole or in part, a moiety selected from:

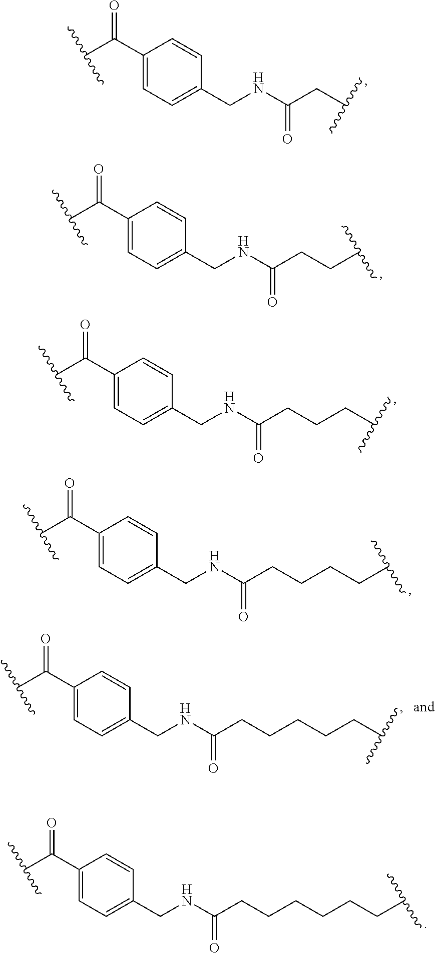 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In some embodiments, the Linker may comprise, either in whole or in part, a moiety selected from:

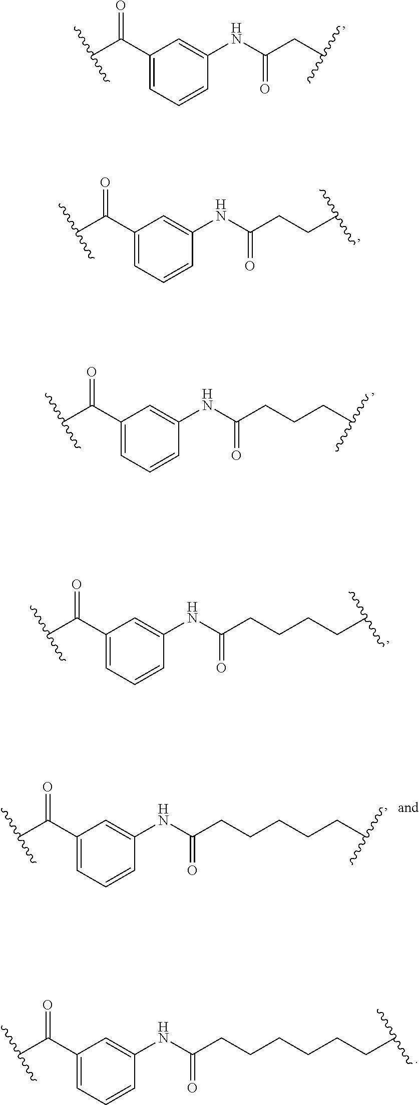 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some embodiments, the Linker may comprise, either in whole or in part, a moiety selected from:

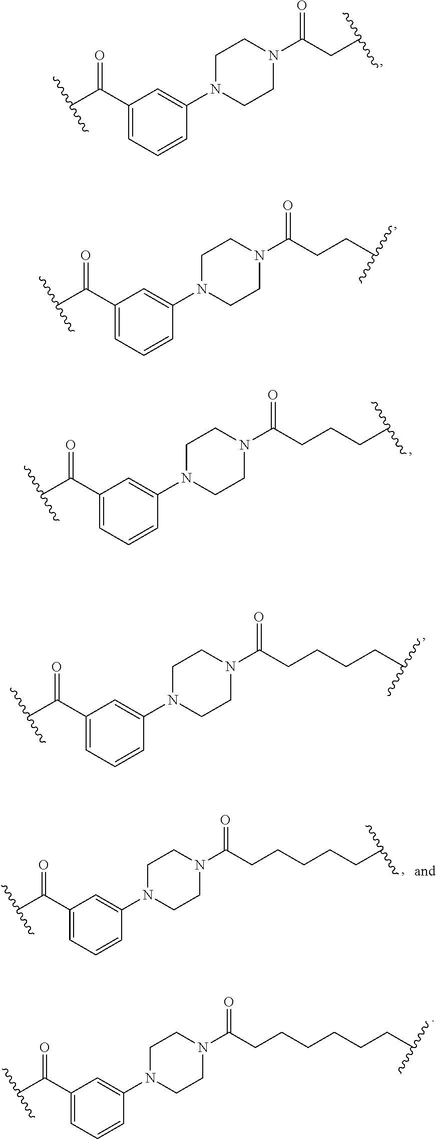 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some embodiments, the Linker may comprise, either in whole or in part, a moiety selected from:

In some embodiments, the Linker may comprise, either in whole or in part, a moiety selected from:

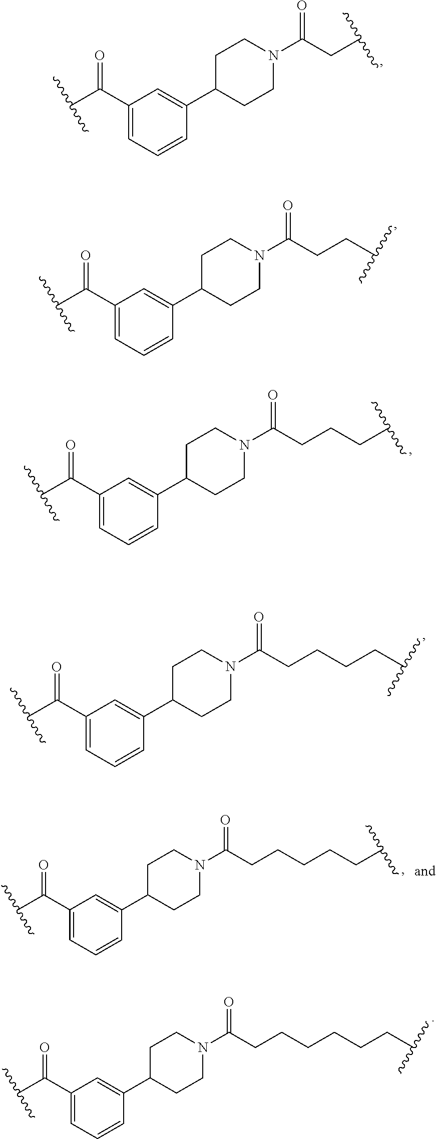 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some embodiments, the Linker may comprise, either in whole or in part, a

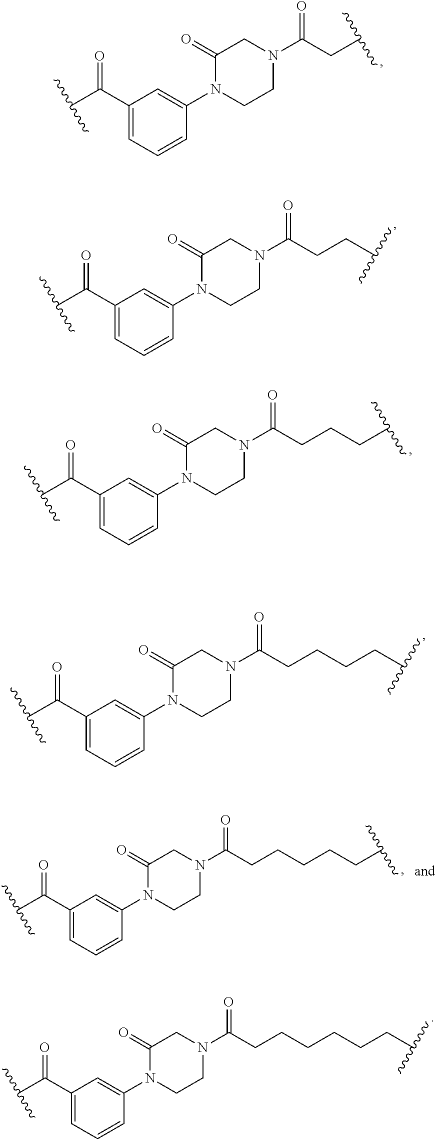 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some embodiments, the Linker may comprise, either in whole or in part, a moiety selected from:

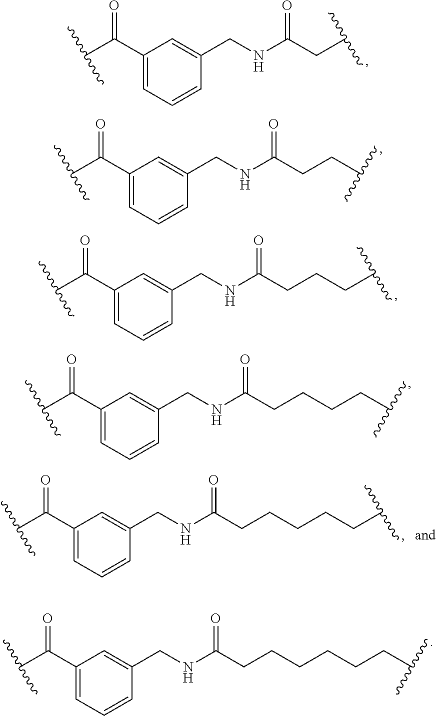 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some embodiments, the Linker may comprise, either in whole or in part, a

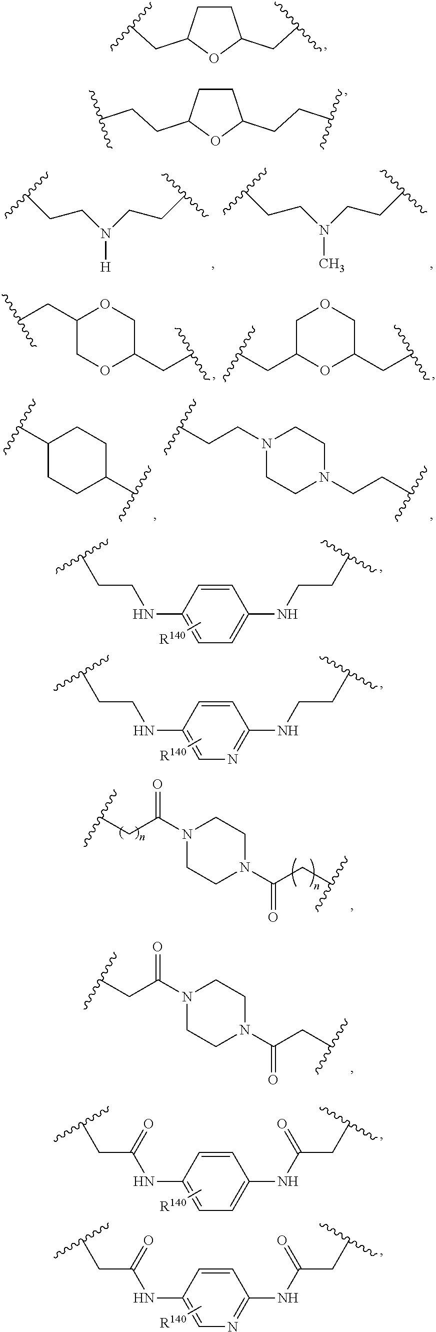

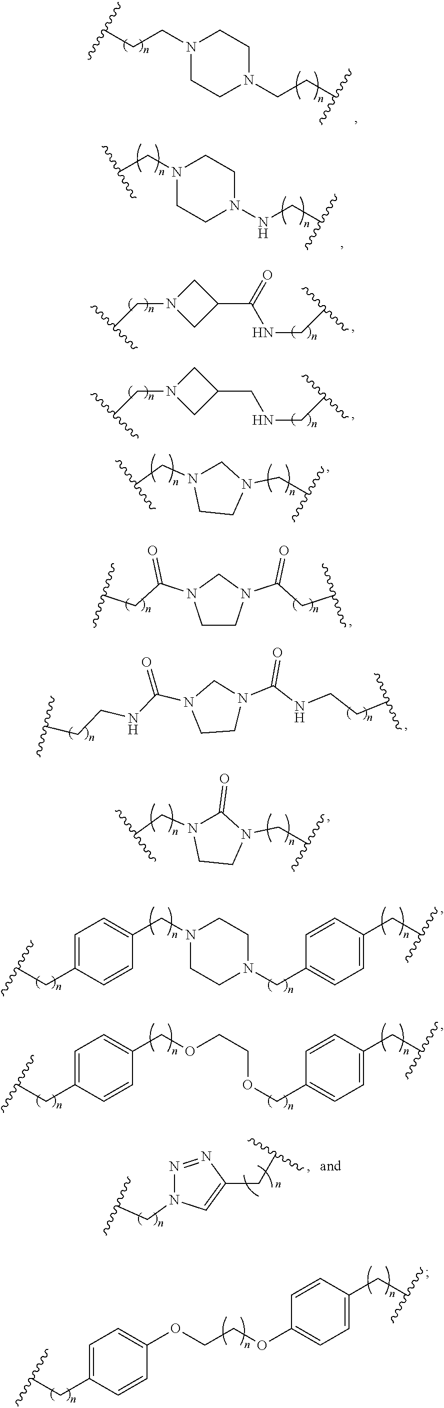

wherein n is independently selected at each occurrence from 1, 2, 3, 4, 5, and 6; and all other variables are as defined herein.

In some embodiments, the Linker may comprise, either in whole or in part, a moiety selected from:

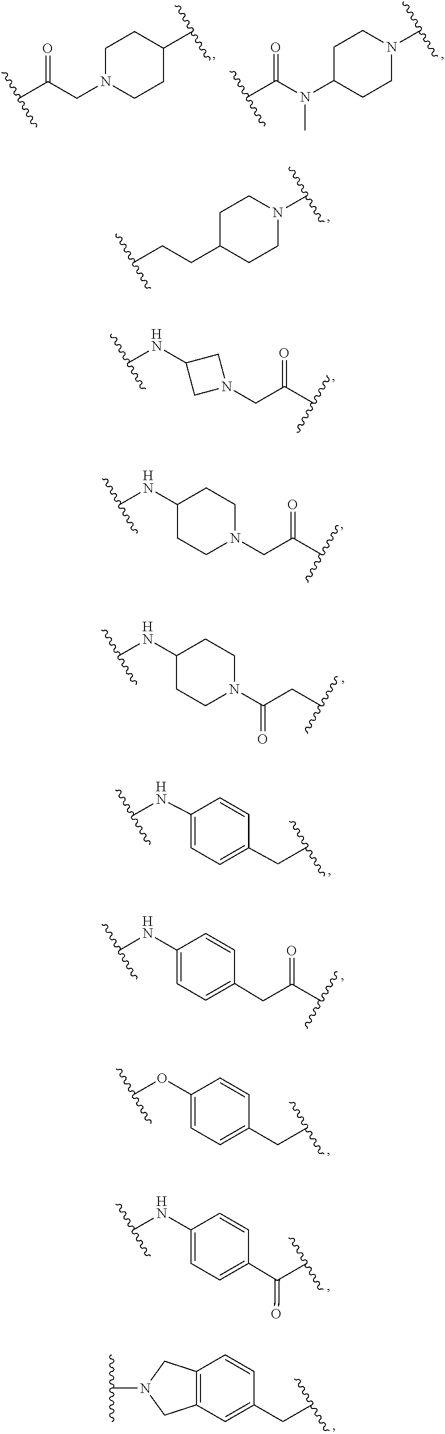

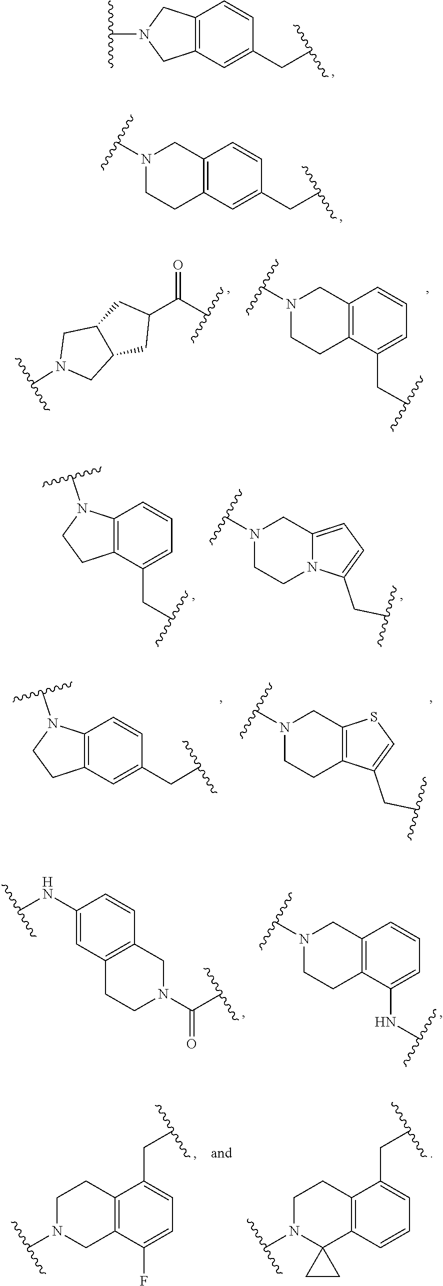

In some embodiments, the Linker may comprise, either in whole or in part, a moiety selected from:

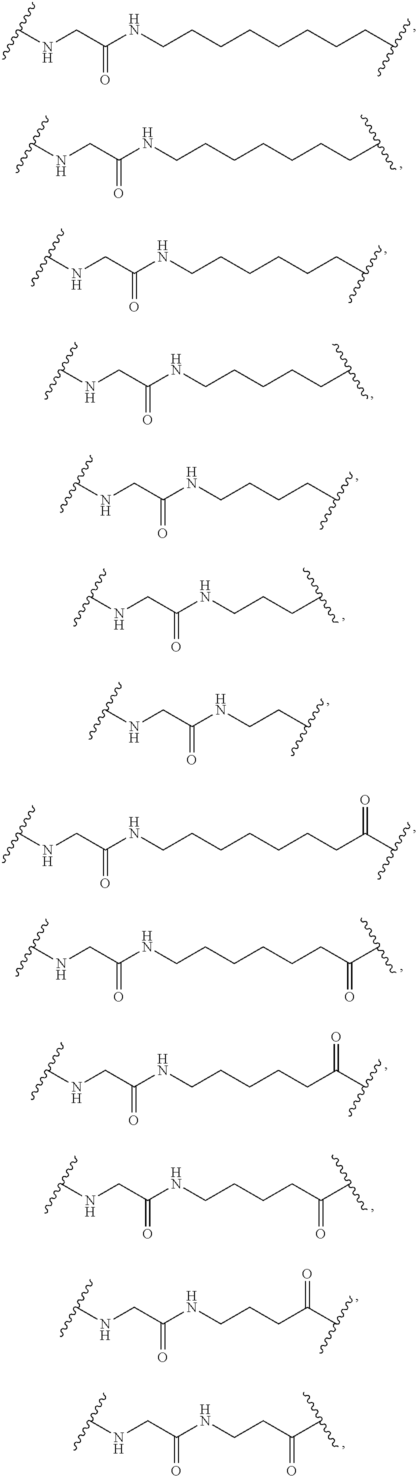

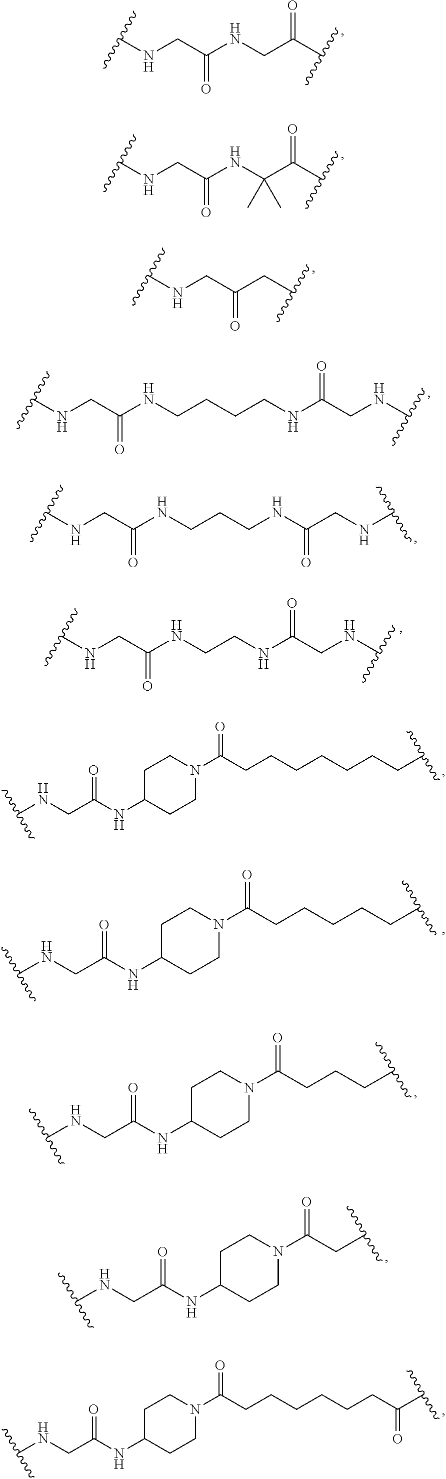

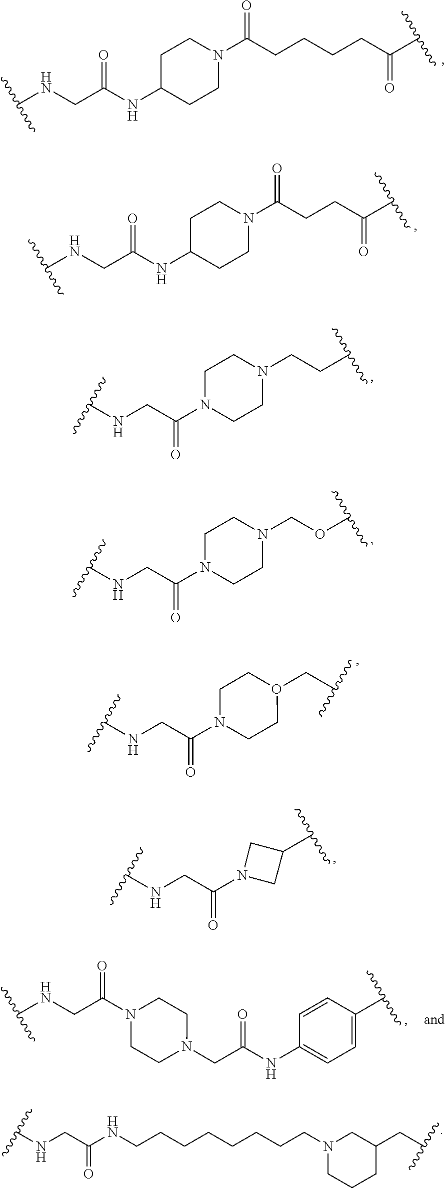

In some embodiments, the Linker may comprise, either in whole or in part, a moiety selected from:

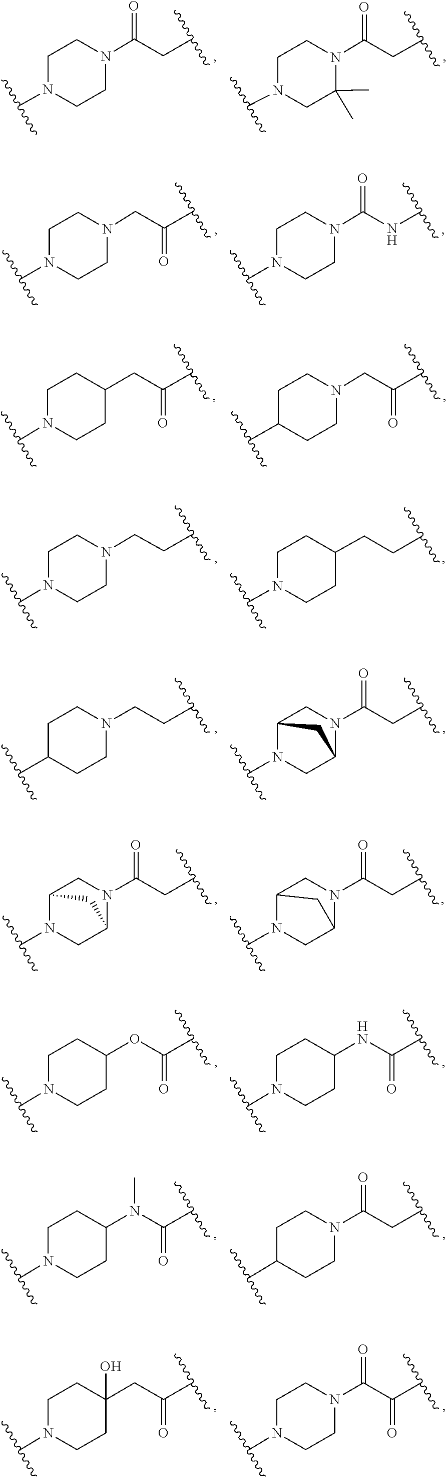

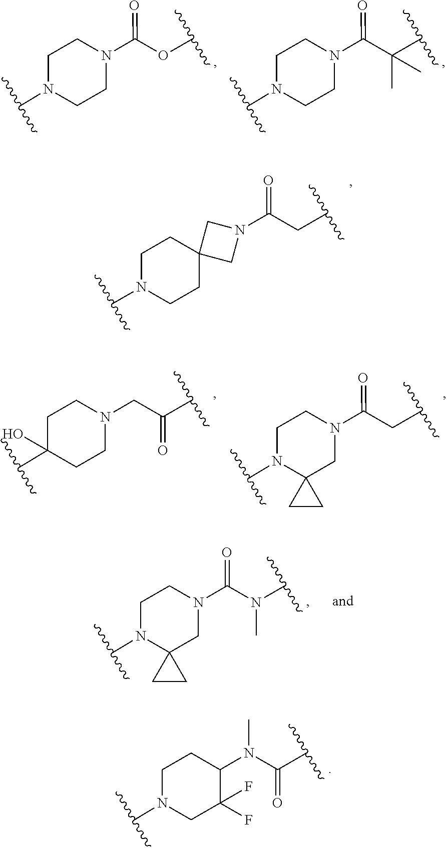

In some embodiments, the Linker may comprise, either in whole or in part, a moiety selected from:

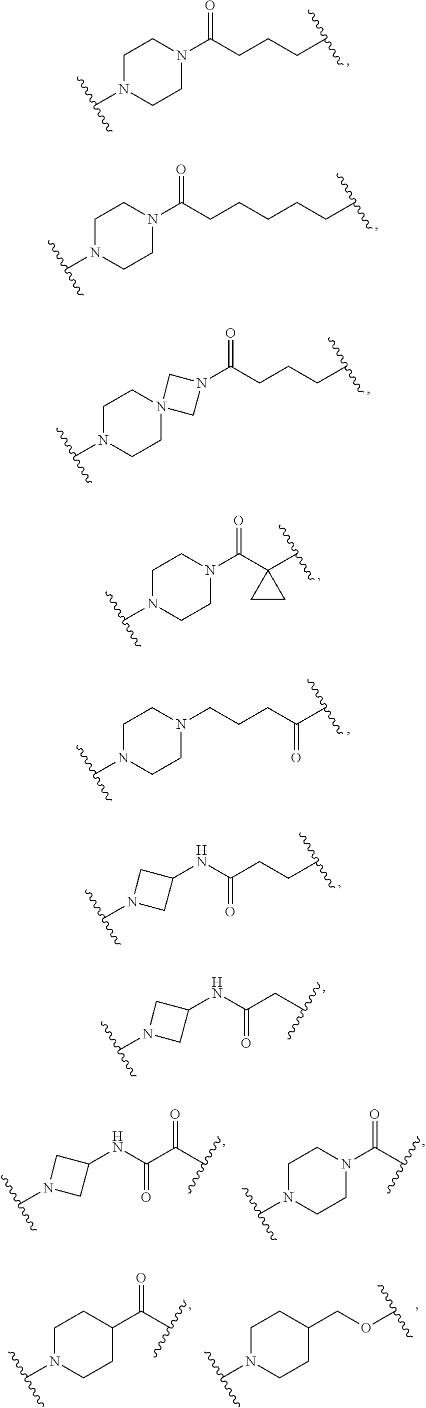

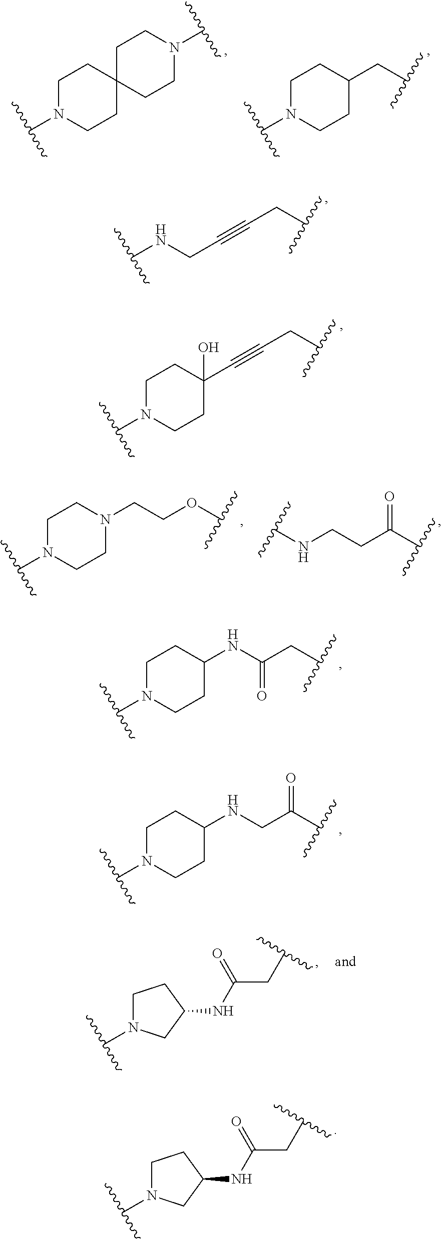

In some embodiments, the Linker may comprise, either in whole or in part, a moiety selected from:

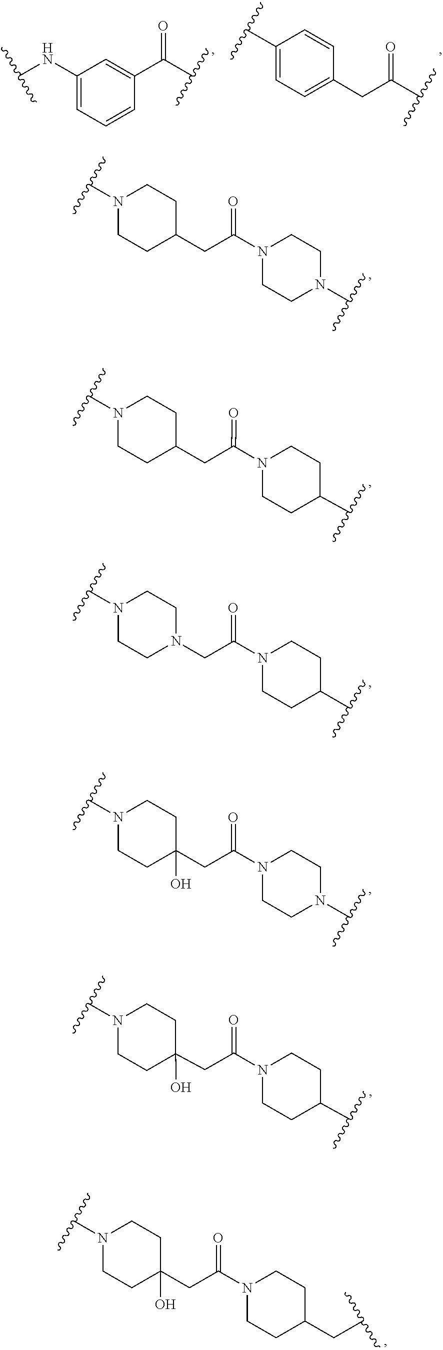

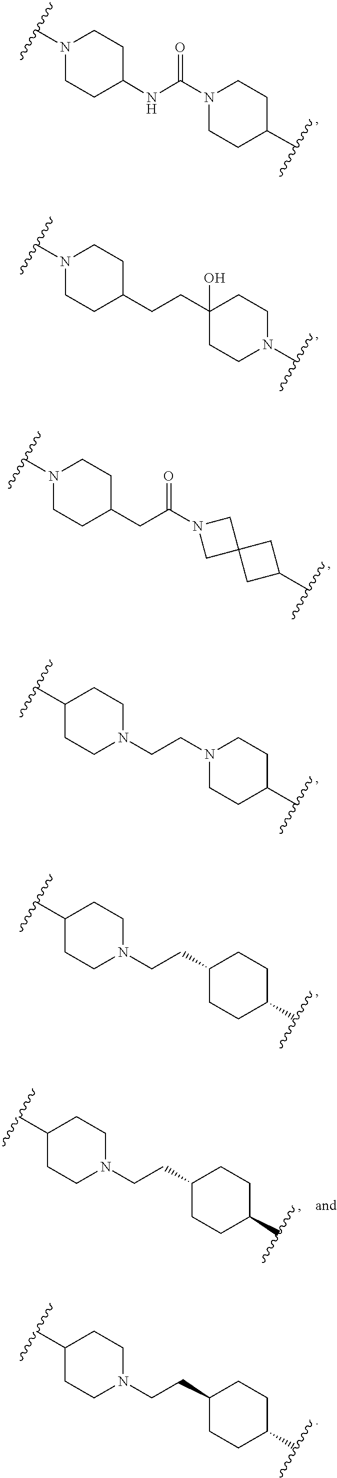

In some embodiments, the linker may comprise, at least in part, a cleavable moiety. In some embodiments, the cleavable moiety may be cleaved under reductive conditions. In some embodiments, the cleavable moiety may be cleaved under oxidative conditions. In some embodiments, the cleavable moiety may be cleaved under acidic conditions. In some embodiments, the cleavable moiety may be cleaved under alkaline conditions. In some embodiments, the cleavable moiety may be cleaved photolytically. In some embodiments, the cleavable moiety may be cleaved by a transition metal catalyst. In some embodiments, the cleavable moiety may be cleaved enzymatically.

In some embodiments, the cleavable moiety may comprise, at least in part, a disulfide (—S—S—) or an azo (—N═N—) moiety. In some embodiments, the disulfide or azo moiety may be cleaved under reductive conditions. In some embodiments, a disulfide or azo moiety may replace the moiety represented by any one of R¹⁰⁰, R¹⁰¹, R¹⁰², R¹⁰³, and R¹⁰⁴ in the linker of Formula L1.

In some embodiments, the cleavable moiety may be cleaved by a protease. In some embodiments, the cleavable moiety may comprise, at least in part, a moiety having the structure:

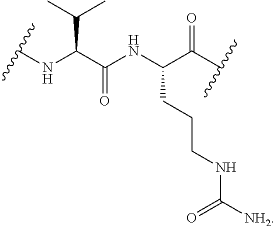 [SMILES: *N[C@H](C(=O)N[C@@H](CCCNC(N)=O)C(*)=O)C(C)C]

In some embodiments, the above moiety may replace the moiety represented by any one of R¹⁰⁰, R¹⁰¹, R¹⁰², R¹⁰³, and R¹⁰⁴ in the linker of Formula L1.

In some embodiments, the cleavable moiety is photocleavable. In some embodiments, the cleavable moiety may comprise, at least in part, a moiety selected from:

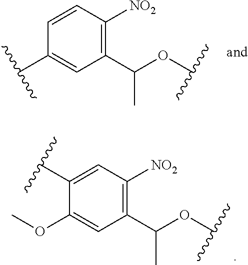 [SMILES: *OC(C)c1cc(*)ccc1[N+](=O)[O-] ; *OC(C)c1cc(OC)c(*)cc1[N+](=O)[O-]]

### 2. Nucleic Acid Delivery

In the methods described above which include the administration and uptake of exogenous DNA into the cells of a subject, the disclosed aptamers can be in the form of naked peptides, DNA, or RNA. The disclosed aptamers can be conjugated to a variety of molecules including small molecules (e.g., cholesterol, bile acids, and lipids), peptides, polymers, proteins (e.g., antibody), to improve the stability, cellular internalization, or cell-specific active targeting delivery

In some embodiments, an aptamer, and/or an aptamer construct is prepared with a carrier that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of an aptamer, and/or an aptamer construct may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

### 3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. Methods of using the compositions.

4. Methods of Detecting the Presence and/or Abundance of a Protein and/or Peptide Target

It is understood and herein contemplated that the disclosed aptamer-handle conjugates can be used for the detection and quantification of any protein and/or peptide target to which an aptamer can be designed to bind. By utilizing a detectable label the disclosed aptamer-handle conjugates can be used as an alternative to Western Blot, quantitative dot blot, immunostaining, ELISA, ELISpot, and flow cytometry for the detection and abundance determination of a protein or peptide target. Accordingly, disclosed herein are methods of detecting a target protein and/or peptide comprising contacting the protein and/or peptide with an aptamer-handle conjugate wherein the aptamer-handle conjugate comprises any of the detectable labels disclosed herein (such as, for example, biotins, a chemiluminescent marker, a fluorescent marker (e.g., fluorophores), radiomarkers, organocatalysts, dye, quantum dot, enzyme, enzyme substrate, and other detectable markers) and assaying for the detectable label on the handle. Thus, in one aspect, disclosed herein are methods of detecting the presence or quantifying the amount of a target protein comprising contacting the target protein with any of the modified aptamer-handle conjugates disclosed herein. For example, disclosed herein are methods of detecting the presence or quantifying the amount of a target protein comprising contacting the target protein with a modified aptamer-handle conjugate comprising an aptamer, an electrophilic leaving group, and a handle; wherein the handle comprises or can be modified with a detectable label (such as, for example, (biotin, a bioconjugation handle, a chemiluminescent marker (such as, for example, horseradish peroxidase), a fluorescent marker, a radiomarker, a dye, a quantum dot, an enzyme, an enzyme substrate, a catalyst, or a small molecule ligand). In some aspects, the method can further comprise measuring the presence, absence or quantity of the bound aptamer relative to a control or standard. Additionally, in some aspects the methods can further comprise transferring the target protein to a membrane after contacting the target protein with the aptamer-handle conjugate.

### 5. Diagnosis and Treatment

In one aspect, disclosed herein are methods for the diagnosis and/or detection of a specific disease state in a tissue or cell sample (including, but not limited to fresh and/or fixed samples such as, for example, formalin fixed paraffin embedded (FFPE) samples) comprising: (a) contacting a tissue sample or cell sample with an aptamer, wherein the aptamer comprises at least one aptamer of any preceding aspect; (b) measuring the presence, absence or quantity of the aptamer; and (c) diagnosing the disease state of the tissue or cell based on the presence, absence or quantity of the aptamer measured. In some aspects, the aptamer has a dissociation half-life from the target of from about 15 minutes and about 240 minutes (including, but not limited to 15, 20, 25, 30, 35, 40, 4 5, 50, 55, 60, 65, 70, 75, 80, 85, 90, 95, 100, 105, 110, 115, 120, 130, 140, 150, 160, 170, 180, 190, 200, 210, 220, 230, or 240 minutes).

6. Methods of Detecting and/or Treating Cardiac Disease

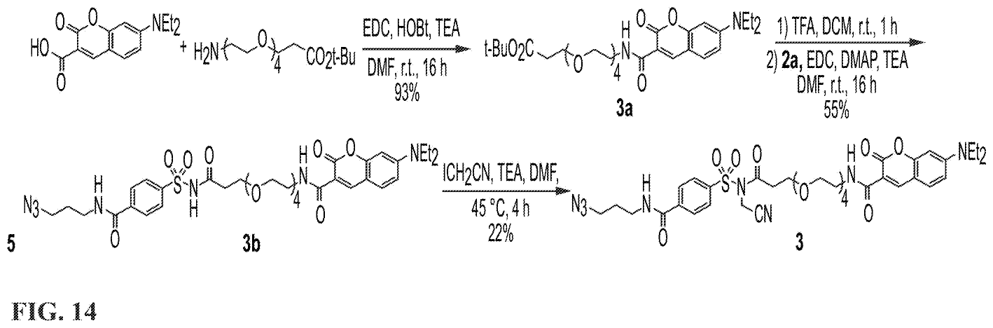

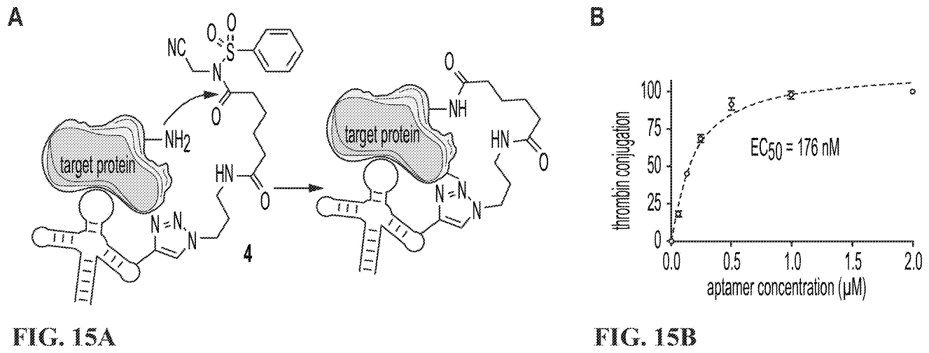

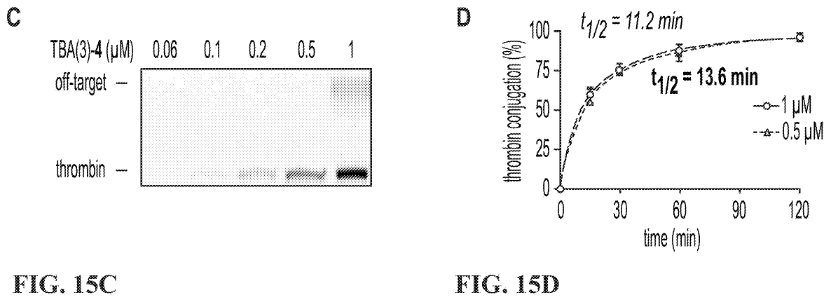

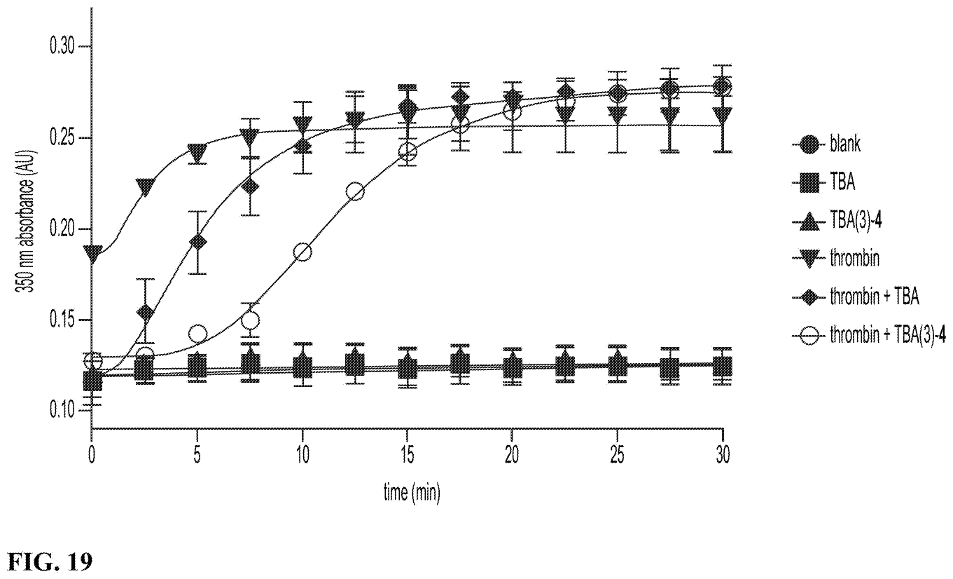

Thrombin, a serine protease, converts fibrinogen to insoluble fibrin, thereby contributing to clotting. Thrombin is a key enzyme in maintaining hemostasis and hyperactivation leads to thrombosis—one of the most common causes of death. Consequently, thrombin misregulation can lead to myocardial infraction, pulmonary embolism, stroke, and venous thrombosis. We selected TBA as the initial target due to its facile synthesis, robust protein binding, and therapeutic potential as an anticoagulant by inhibiting thrombin's fibrinogen binding site with nanomolar affinity. As shown herein, the disclosed aptamers have particular selectivity for thrombin. and can deliver a detectable handle to thrombin. To assess enzymatic activity, we capitalized on thrombin's cleavage of fibrinogen into insoluble fibrin, which can be quantified via absorbance at 350 nm. Fibrinogen was added and absorbance was recorded over 30 min (FIG. 19), followed by extrapolation of clotting times (FIG. 15E). Compared to unmodified TBA, TBA(3)-4 lead to a 2-fold prolonged clotting time, demonstrating that the potency of an aptamer can be enhanced through covalent crosslinking. Thus, in one aspect disclosed herein are methods of diagnosing and/or detecting a circulatory condition (such as, for example, thrombosis, thromboembolism, Paget-Schroetter disease, fibrosis, stroke, or myocardial infarction) in a subject comprising a) contacting a tissue sample or cell sample with any of the modified aptamer handle complexes conjugates disclosed herein; (b) measuring the presence, absence or quantity of the aptamer; and (c) diagnosing and/or detecting the disease state of the tissue or cell based on the presence, absence or quantity of the aptamer measured. For example, in one aspect, disclosed herein are methods of diagnosing and/or detecting a circulatory condition (such as, for example, thrombosis, thromboembolism, Paget-Schroetter disease, fibrosis, stroke, or myocardial infarction) in a subject comprising a) contacting a tissue sample or cell sample with a modified aptamer-handle conjugate comprising a modified aptamer that targets thrombin (such as, for example, SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8), an electrophilic leaving group (such as, for example, N-acyl sulfonamide cleavable electrophile or tosyl cleavable electrophile) for crosslinking a target or a modified aptamer-handle conjugate, and a detectable handle (such as, for example, biotins, a chemiluminescent marker, a fluorescent marker (e.g., fluorophores), radiomarkers, organocatalysts, dye, quantum dot, enzyme, enzyme substrate, and other detectable markers). In one aspect, the method can further comprise obtaining a tissue or cell sample from the subject.

It is understood and herein contemplated that by replacing a detectable label with a handle that can break up a thrombus or fibrotic legion, the disclosed aptamer-handles conjugates and modified aptamers can be used to treat cardiac diseases. Thus, disclosed herein are methods of treating, inhibiting, reducing, decreasing, ameliorating, and/or preventing a circulatory condition (such as, for example, thrombosis, thromboembolism, Paget-Schroetter disease, fibrosis, stroke, or myocardial infarction) in a subject comprising administering to the subject any of the modified aptamers and/or modified aptamer handle conjugates (such as, for example, a modified aptamer-handle conjugates comprising an aptamer that binds a thrombin proteins including, but not limited to, an aptamer as set forth in SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8) disclosed herein. In one aspect, the modified aptamer-handle conjugate can comprise an aptamer that targets thrombin (such as, for example, SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8), and an electrophile (such as, for example, N-acyl sulfonamide cleavable electrophile or tosyl cleavable electrophile).

7. Methods of Detecting and/or Treating a Coronavirus Infection

Having shown that detection of a target protein is possible using the disclosed aptamer-handle conjugates or modified aptamers, the same principle applies to detection of other disease conditions once a target protein and selective aptamer are identified. In one aspect, disclosed herein are methods of detecting and/or diagnosis a coronavirus infection (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) comprising a) contacting a tissue sample or cell sample with any of the modified aptamer handle complexes conjugates disclosed herein, wherein the aptamer selectively binds the coronal virus spike protein or the receptor binding domain of the coronavirus spike or nucleocapsid protein (such as, for example, the aptamer as set forth in SEQ ID NO: 9, SEQ ID NO: 10, SEQ ID NO: 11, SEQ ID NO: 12, SEQ ID NO: 13, SEQ ID NO: 14, and/or SEQ ID NO: 15); (b) measuring the presence, absence or quantity of the aptamer; and (c) diagnosing and/or detecting the a coronavirus infection in based on the presence, absence or quantity of the aptamer measured. For example, disclosed herein are methods of detecting and/or diagnosis a coronavirus infection (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) comprising a) contacting a tissue sample or cell sample with an aptamer-handle conjugate comprising i) an aptamer wherein the aptamer selectively binds the coronal virus spike protein or the receptor binding domain of the coronavirus spike protein or nucleocapsid or the receptor binding domain of the coronavirus spike protein or nucleocapsid (such as, for example, the aptamer as set forth in SEQ ID NO: 9, SEQ ID NO: 10, SEQ ID NO: 11, SEQ ID NO: 12, SEQ ID NO: 13, SEQ ID NO: 14, and/or SEQ ID NO: 15), ii) and electrophile (such as, for example, N-acyl sulfonamide cleavable electrophile or tosyl cleavable electrophile) for crosslinking a target, and iii) a detectable handle (such as, for example, biotins, a chemiluminescent marker, a fluorescent marker (e.g., fluorophores), radiomarkers, organocatalysts, dye, quantum dot, enzyme, enzyme substrate, and other detectable markers); (b) measuring the presence, absence or quantity of the aptamer; and (c) diagnosing and/or detecting the a coronavirus infection in based on the presence, absence or quantity of the aptamer measured. In one aspect, the method can further comprise obtaining a tissue or cell sample from the subject.

Also disclosed herein are methods of treating, inhibiting, reducing, decreasing, ameliorating, and/or preventing a coronavirus infection (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) comprising administering to a subject with a coronavirus infection any of the modified aptamers and/or modified aptamer handle conjugates disclosed herein (including, but not limited to aptamer-handle conjugates and/or modified aptamers, wherein the aptamer selectively binds the coronal virus spike protein or the receptor binding domain of the coronavirus spike or nucleocapsid protein (such as, for example, the aptamer as set forth in SEQ ID NO: 9, SEQ ID NO: 10, SEQ ID NO: 11, SEQ ID NO: 12, SEQ ID NO: 13, SEQ ID NO: 14, and/or SEQ ID NO: 15)).

8. Method of Detecting and/or Treating Cancer

In one aspect, it is understood and herein contemplated that the disclosed compositions can be used to detect, treat, inhibit, reduce, decrease, ameliorate, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, retinoblastoma, gastric cancers, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. In one aspect disclosed herein are methods of treating, inhibiting, reducing, decreasing, ameliorating, and/or preventing a cancer and/or metastasis (such as, for example, retinoblastoma, colon cancer, lung cancer, and/or gastric cancers) in a subject.

In one aspect disclosed herein are methods of diagnosing and/or detecting a cancer and/or metastasis (such as, for example, retinoblastoma, colon cancer, lung cancer, and/or gastric cancers) in a subject comprising contacting a tissue sample or cell sample with any of the modified aptamer handle conjugates disclosed herein (such as, for example an aptamer-handle conjugates comprising an aptamer as set forth in SEQ ID NO: 16 or SE ID NO: 17); (b) measuring the presence, absence or quantity of the aptamer; and (c) diagnosing and/or detecting the disease state of the tissue or cell based on the presence, absence or quantity of the aptamer measured. For example, disclosed herein are methods of diagnosing and/or detecting a cancer and/or metastasis (such as, for example, retinoblastoma, colon cancer, lung cancer, and/or gastric cancers) in a subject comprising contacting a tissue sample or cell sample with an aptamer-handle conjugates comprises an aptamer ((such as, for example an aptamer-handle conjugate comprising an aptamer as set forth in SEQ ID NO: 16 or SEQ ID NO: 17), an electrophile (such as, for example, N-acyl sulfonamide cleavable electrophile or tosyl cleavable electrophile) for crosslinking a target, and a handle (including but not limited to detectable labels (for detection and/or imaging of the cancer such as, for example, biotins, a chemiluminescent marker, a fluorescent marker (e.g., fluorophores), radiomarkers, organocatalysts, dye, quantum dot, enzyme, enzyme substrate, and other detectable markers)); (b) measuring the presence, absence or quantity of the aptamer or label; and (c) diagnosing and/or detecting the disease state of the tissue or cell based on the presence, absence or quantity of the aptamer measured. In one aspect, the method can further comprise obtaining a tissue or cell sample from the subject.

It is understood and herein contemplated that the disclosed modified aptamer-handle conjugates can not only detect a cancer, but can be used for treatment. Accordingly, disclosed herein are methods of treating, inhibiting, reducing, decreasing, ameliorating, and/or preventing a cancer and/or metastasis (such as, for example, retinoblastoma, colon cancer, lung cancer, and/or gastric cancers) in a subject comprising administering to the subject any of the modified aptamer-handle conjugates disclosed herein. For example, disclosed herein are methods of treating, inhibiting, reducing, decreasing, ameliorating, and/or preventing a cancer and/or metastasis (such as, for example, retinoblastoma, colon cancer, lung cancer, and/or gastric cancers) in a subject comprising administering to the subject an aptamer-handle conjugate, wherein the aptamer-handle conjugate comprises an aptamer ((such as, for example an aptamer-handle conjugate comprising an aptamer as set forth in SEQ ID NO: 16 or SEQ ID NO: 17), an electrophile (such as, for example, N-acyl sulfonamide cleavable electrophile or tosyl cleavable electrophile) for crosslinking a target, and a handle (including but not limited to detectable labels (for detection and/or imaging of the cancer such as, for example, biotins, a chemiluminescent marker, a fluorescent marker (e.g., fluorophores), radiomarkers, organocatalysts, dye, quantum dot, enzyme, enzyme substrate, and other detectable markers) and/or a small molecule anti-cancer agent or other anti-cancer agents for treatment)).

It is understood and herein contemplated that in some aspects, the method of detecting, diagnosing, treating, inhibiting, reducing, decreasing, ameliorating and/or preventing a cancer and/or metastasis can further comprise the administration of any of the aptamers disclosed herein alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aligopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### 9. Methods of Recruiting Immune Cells to a Target Microenvironment

In one aspect, it is understood and herein contemplated that the disclosed aptamer-handle conjugates can specifically bind protein and/or peptide targets at the site of an infection or tumor microenvironment. By using a handle that attracts or stimulates immune cells, the aptamer-handle conjugate can be used to direct immune cells (including, but not limited to effector CD8 T cells, central memory CD8 T cells, peripheral memory CD8 T cells, CD4 T cells, Natural Killer (NK) cells, NK T cells, macrophage, chimeric antigen receptor (CAR) T cells, CAR NK cells, CAR Macrophage (CARMA), activated B cells and/or memory B cells) to a target microenvironment. In one aspect, the handle can comprise proinflammatory chemokines such as, for example, CCL2, CCL3, CCL4, CCL5, CCL7, CCL8, CCLJ1, CCL13, CCL17, CCL22, CCL24, CCL26, CXCL8, CXCL9, CXCL10, CXCL11. As the cell type attracted by a given chemokine are known, the immune cells attracted can be tailored to the response desired. For example, T cells can be attracted by an aptamer-handle conjugate comprising a handle comprising CCL1, CCL2, CCL17, CCL22, CXCL9, CXCL10, and CXCL11; macrophage can be attracted by an aptamer-handle conjugate comprising a handle comprising. CCL2, CCL3, CCL5, CCL7, CCLSCCL13, CCL17, and CCL22. Additionally, an aptamer-handle conjugate comprising a handle comprising inflammatory cytokines including by not limited to Il-1, IIL-2, IL-3, IL-4, IL-5, L-6, IL-7, IL-8, IL-17, TNF-α, TNF—B, IFN-α, IFN-γ, GM-CSF, and G-CSF. Furthermore, immune cells, including T cells and NK cells, can be engineered to recognize and be activated by small molecule handles, such as biotin and benzylguanine, covalently attached to tumor cell surfaces through covalent aptamers. In other aspects, the handle can comprise an designed target antigen for an antigen binding molecule such as, for example, an antibody, scFV, nanobody, diabody, CAR T cell, CAR NK cell, and/or CARMA. Accordingly, in one aspect disclosed herein are methods of attracting immune cells to a target microenvironment (such as a tumor microenvironment or site of infection or injury) comprising delivering an aptamer-handle conjugate to the target microenvironment; wherein the aptamer-handle conjugate comprises an aptamer that binds to a target protein and/or peptide and the handle comprises a chemokine, inflammatory cytokine, and/or a target antigen.

### 10. Methods of Delivering a Handle to a Target Protein

In one aspect, disclosed herein are methods of delivering a handle to a target protein (such as biotin, a bioconjugation handle, a fluorescent marker, radiomarker, organocatalyst, or small molecule ligand, PROTAC® (E3 ligase ligand), or LYTAC® (cation-independent mannose-6-phosphate receptor (CI-M6PR) ligand) to a target cell comprising conjugating a labeled electrophile to an aptamer creating an aptamer-handle conjugate; and contacting the target cell with said aptamer-handle conjugate. As the handle can be a detectable label, also disclosed herein are methods of labeling one or more protein targets on or in a cell (including but not limited to intracellular protein targets and protein targets on the cell membrane) comprising conjugating a labeled electrophile to an aptamer creating an aptamer-handle conjugate; and contacting the cell with the aptamer electrophile conjugate.

### C. METHODS OF MAKING APTAMER HANDLE CONJUGATES AND MODIFIED APTAMERS

In one aspect, disclosed herein are methods of making aptamer-handle conjugate comprising a) constructing a nucleic acid sequence via solid-phase oligonucleotide synthesis; b) replacing one or more thymidines with triisopropylsilyl (TIPS) protected 5-ethynyl-2′-deoxyuridine (EdU) or 5-(octa-1,7-diynyl)-2′-deoxyuridine (OdU) phosphoramidite; and c) conjugating an electrophile (such as, for example, N-acyl sulfonamide cleavable electrophile or tosyl cleavable electrophile) and handle to the aptamer via, for example, copper catalyzed [3+2]cycloaddition. It is understood and herein contemplated that the reaction for the electrophile and handle bioconjugation to the nucleic acid aptamer can include any means known in the art, including, but not limited to disulfide exchange, nucleophilic substitution of halo acetamides with thiols, thiol-maleimide conjugation, NHS-ester conjugation to amines, sulfonyl chloride conjugations to amines, isocyanate conjugation to amines, isothiocyanate conjugation to amines, aldehyde conjugation to amines through reductive amination, amine conjugation through transition-metal catalyzed reductive alkylation, thiolene reaction, copper-catalyzed azide-alkyne [3+2] cycloaddition, strain-promoted azide-alkyne [3+2] cycloaddition, aldehyde-aniline conjugation through a Mannich reaction, palladium-catalyzed allylation, glyoxylate-amine conjugation, native chemical ligation, Suzuki coupling, Sonagashira coupling, Heck coupling, inverse electro demand Diels-Alder reaction, tetrazine ligation, oxime ligation, Staudinger ligation, traceless Staudinger ligation, photoclick 1,3-dipolar cycloadditions, [4+1] Isonitrile cycloadditions, 2-acylboronic acid condensation, hydrazone formation, and olefin metathesis. In some aspects, the electrophile to the aptamer occurs via the aryl sulfonamide of the electrophile. In some aspects, the conjugation of the electrophile to the aptamer occurs via the amide motif of the electrophile.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Modification of Native Proteins Through Covalent Aptamers

### a) Results and Discussion

Covalent modification of proteins via bioorthogonal reactions is essential for many biological investigations and the development of protein-based therapeutics. Common approaches to generate protein conjugates include metabolic labeling, activity-based labeling, fusion of the protein of interest to an enzyme or peptide tag, and unnatural amino acid incorporation. Specific covalent modification of native proteins in their biological environment has been challenging. One approach has been the modification of small molecule inhibitors and ligands with electrophilic “warheads”. In contrast to traditional, reversible protein-ligand interactions, a covalent ligand is designed such that the reversible association is followed by proximity-driven irreversible crosslinking between a strategically placed electrophile on the ligand and a nucleophilic residue on the target protein. Small molecule covalent ligands offer several advantages over their non-covalent counterparts, including the ability to outcompete endogenous ligands with similar affinities, dosage at lower concentration, and a duration of action that matches the protein turnover rate. Prominent examples include ibrutinib and afatinib, FDA-approved inhibitors that covalently crosslink to Bruton's tyrosine kinase and epidermal growth factor receptor, respectively. In a complementary approach, small molecule ligands bearing an electrophilic warhead capable of transferring a functional label to the target protein were developed. Like covalent inhibitors, these electrophiles are escorted to a nucleophilic residue by a protein binding ligand. In contrast, however, the protein ligand is released after label (e.g., biotin) transfer. This approach enables specific modification of native proteins and was utilized, e.g., to covalently modify carbonic anhydrase with a ¹⁹F NMR probe to measure inhibitor binding in live cells.

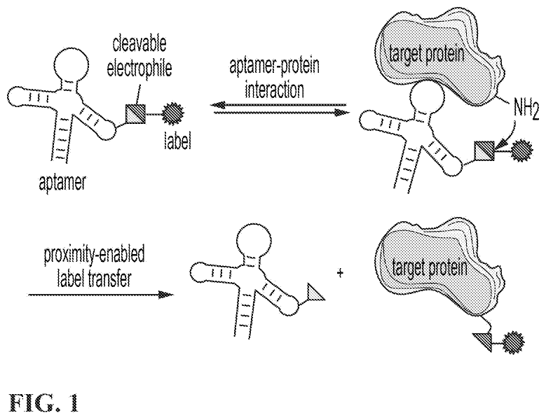

Here, we describe the first examples of aptamers modified with cleavable electrophiles that provide transfer of chemical motifs to a protein (FIG. 1). The aptamer serves as an affinity ligand for a protein target, bringing its conjugated electrophile in the vicinity of a nucleophilic residue, e.g., lysine, followed by selective label transfer to the protein through covalent bond formation. The aptamer is cleaved from the electrophile at the same time as the covalent crosslink is formed and can then dissociate from its target. In this first study demonstrating the overall viability of this concept, we investigated the installation of electrophilic warheads into the thrombin binding aptamer, TBA, a 15-nucleotide DNA molecule. Thrombin, a serine protease, converts fibrinogen to insoluble fibrin, thereby contributing to clotting. Consequently, thrombin misregulation can lead to myocardial infraction, pulmonary embolism, stroke, and venous thrombosis. We selected TBA as the initial target due to its facile synthesis, robust protein binding, and therapeutic potential as an anticoagulant by inhibiting thrombin's fibrinogen binding site with nanomolar affinity.

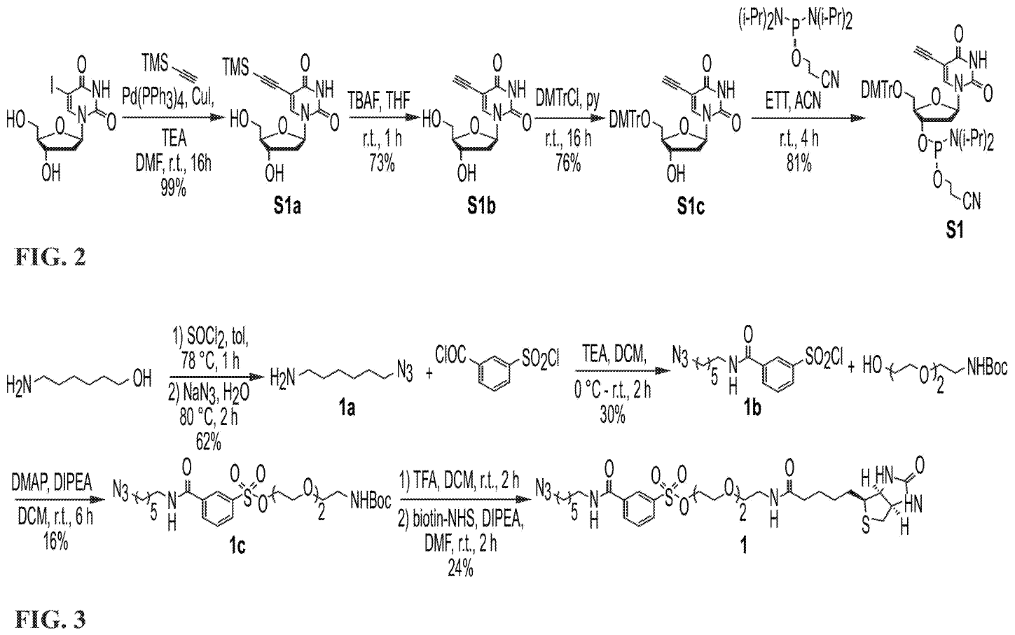

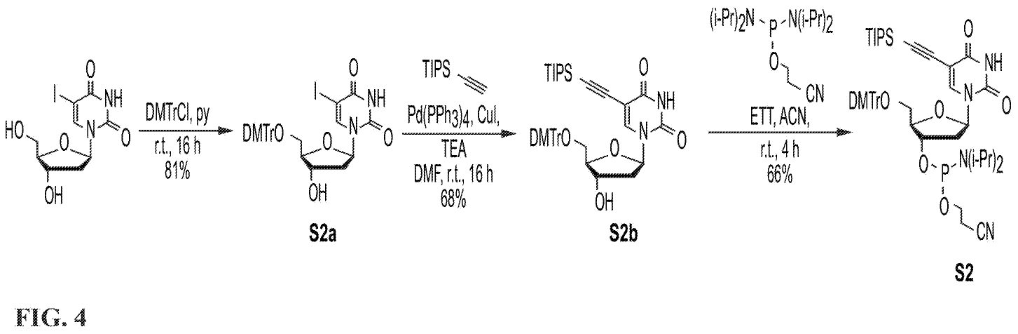

Electrophilic aptamers were generated by incorporating alkyne-modified phosphoramidites into TBA via solid-phase oligonucleotide synthesis and conjugated with the electrophilic warheads. Electrophiles could not be directly incorporated due to their sensitivity to basic conditions required to remove nucleobase protecting groups post-oligomerization. The modified nucleotide 5-ethynyl-2′-deoxyuridine (EdU) phosphoramidite was synthesized (FIG. 2). By individually synthesizing each modified aptamer, the six thymidines on TBA (5′-GGTTGGTGTGGTTGG-3′)(SEQ ID NO: 1) were systematically replaced with EdU at each residue or combination of residues to screen for positions best suited for selective thrombin modification without disrupting the aptamer structure (Table 1). Oligonucleotides were purified by PAGE and characterized by MALDI (Table 2). The azido-modified tosyl electrophile 1 capable of transferring a biotin handle was synthesized in four steps (FIG. 3). Biotin was chosen as the first transferrable handle since it enables both sensitive detection and facile enrichment of the targeted protein. Each alkyne-bearing aptamer was then individually conjugated to the cleavable electrophile and handle through a copper-catalyzed [3+2]cycloaddition that yielded ˜60% conversion and purified by HPLC. This conjugation strategy was chosen over other common methods (e.g., amine-NHS ester coupling) since it is compatible with the electrophile intended for protein conjugation (e.g., tosylate). Conjugation yields were modest due to partial hydration of the ethynyl group to a methyl ketone during oligonucleotide synthesis and basic deprotection. We addressed this problem by employing a triisopropylsilyl (TIPS) protected EdU phosphoramidite, synthesized in three steps from 5-iodo-2′-deoxyuridine (FIG. 4). After oligonucleotide synthesis and basic deprotection, the TIPS group was removed through a brief exposure to TBAF. Subsequent bioconjugation via [3+2] cycloaddition resulted in >95% conversion.

TABLE 1

| protein target | aptamer | aptamer sequence (5′→3′) |
|---|---|---|
| thrombin | TBA | GGTTGGTGTGGTTGG (SEQ ID NO: 1) |
| thrombin | mTBA | GGT-5′-5′TGGTGTGGTTGG-3 (SEQ ID NO: 2) |
| thrombin | HD22 | AGTCCGTGGTAGGGCAGGTTGGGGTGACT (SEQ ID NO: 3) |
| thrombin | Toggle 25t | GAACAAAGCUGAAGUACUUACCCAAGAUCAUCCCGAACG |
| | | A (SEQ ID NO: 4) |
| thrombin | NU172 | CGCCTAGGTTGGGTAGGGTGGTGGCG (SEQ ID NO: 5) |
| thrombin | RE31 | GTGACGTAGGTTGGTGTGGTTGGGGCGTCACC (SEQ ID |
| | | NO: 6) |
| thrombin | RA36 | GGTTGGTGTGGTTGGTGGTTGGTGTGGTTGG (SEQ ID NO: |
| | | 7) |
| thrombin | M08 | AGCAGCACAGAGGTCAGATGATGGGGATGGGGGGTTGGA |
| | | GGAATGGATGACCTATGCGTGCTACCGTGAA (SEQ ID NO: |
| | | 8) |
| SARS-CoV-2 | RBA | CAGCACCGACCTTGTGCTTTGGGAGTGCTGGTCCAAGGG |
| RBD | | CGTTAATGGACA (SEQ ID NO: 9) |
| SARS-CoV-2 | RBD 4C | ATCCAGAGTGACGCAGCATTTCATCGGGTCCAAAAGGGG |
| RBD | | CTGCTCGGGATTGCGGATATGGACACGT (SEQ ID NO: 10) |
| SARS-CoV-2 | SBA | GGGAGAGGAGGGAGATAGATATCAACCCATGGTAGGTAT |
| S protein | | TGCTTGGTAGGGATAGTGGGCTTGATGTTTCGTGGATGC |
| | | CACAGGAC (SEQ ID NO: 11) |
| SARS-CoV-2 | SP6 | GGGAGAGGAGGGAGATAGATATCAACCCATGGTAGGTAT |
| S protein | | TGCTTGGTAGGGATAGTGGGCTTGATGTTTCGTGGATGC |
| | | CACAGGAC (SEQ ID NO: 12) |
| SARS-CoV-2 | nucleocapsid | GCAATGGTACGGTACTTCCGGATGCGGAAACTGGCTAAT |
| nucleocapsid | binding aptamer 1 | TGGTGAGGCTGGGGCGGTCGTGCAGCAAAAGTGCACGCT |
| protein | | ACTTTGCTAA (SEQ ID NO: 13) |
| SARS-CoV-2 | nucleocapsid | GCAATGGTACGGTACTTCCGGATGCGGAAACTGGCTAAT |
| nucleocapsid | binding aptamer 2 | TGGTGAGGCTGGGGCGGTCGTGCAGCAAAAGTGCACGCT |
| protein | | (SEQ ID NO: 14) |
| SARS-CoV-2 | nucleocapsid | GCAATGGTACGGTACTTCCGGATGCGGAAACTGGCTAAT |
| nucleocapsid | binding aptamer 3 | TGGTGAGGCTGGGGCGGT (SEQ ID NO: 15) |
| protein | | |
| PTK7 | P7BA | ATCTAACTGCTGCGCCGCCGGGAAAATACTGTACGGTTA |
| | | GA (SEQ ID NO: 16) |
| PTK7 | Sgc8 | ATACCAGCTTATTCAATTAGTCACACTTAGAGTTCTAACT |
| | | GCTGCGCCGCCGGGAAAATACTGTACGGTTAGATAGTAA |
| | | GTGCAATCT (SEQ ID NO: 17) |
| PSMA | PBA | TAATACGACTCACTATAGGGACCGAAAAAGACCTGGCTT |
| | | CTATACTAAGTCTACGTTCCC (SEQ ID NO: 18) |
| FOXM1 | FBA | TCACTTCACACCGCATCTCTACGTCCGGTTGCGCTTTCCTT |
| | | T (SEQ ID NO: 19) |
| IgG1 | ABA | GGAGGUGCUCCGAAAGGAACUCC (SEQ ID NO: 20) |
| c-MET | SL1 | ATCAGGCTGGATGGTAGCTCGGTCGGGGTGGGTGGGTTGG |
| | | CAAGTCTGAT-3′ (SEQ ID NO: 25 |

Protein targets and their aptamers. Aptamers can be individually synthesized to contain a single alkyne modification at each bolded position and conjugated to electrophilic warheads post-synthesis.

TABLE 2

| Oligonucleotide | Calculated mass | Measured mass |
|---|---|---|
| TBA(3)-alkyne | 4736.1 | 4734.0 |
| TBA(3)-1 | 5419.4 | 5416.7 |
| TBA(3)-2 | 5284.2 | 5282.6 |
| TBA(3)-3 | 5548.4 | 5547.9 |
| TBA(3)-4 | 5142.2 | 5140.7 |
| 3′fluorescein-TBA(3)-4 | 5572.2 | 5577.9 |

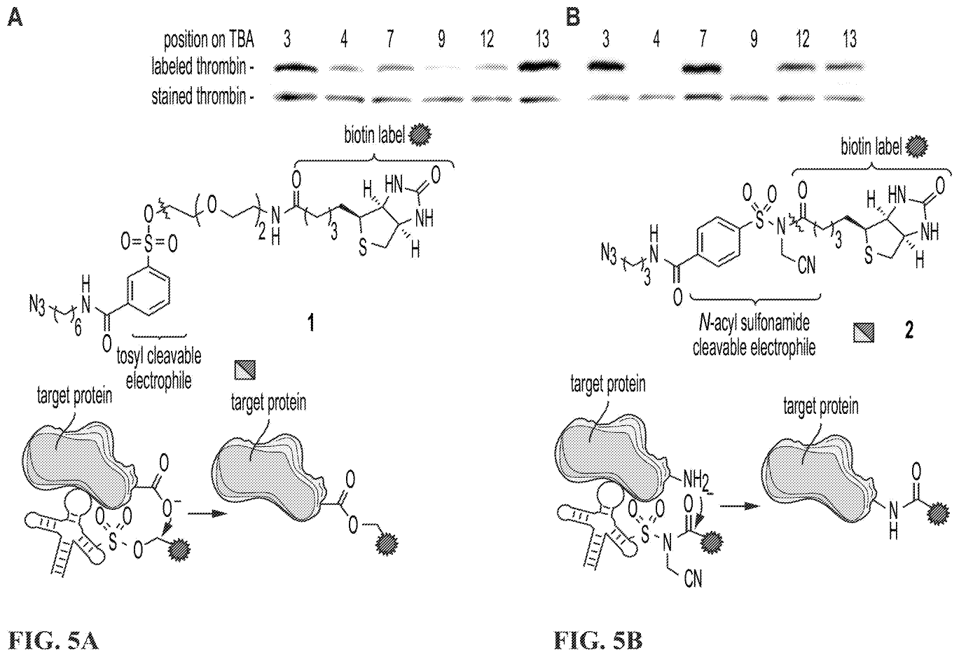

Aptamers modified with the tosyl electrophile 1, a warhead that has broad reactivity toward His, Lys, Tyr, Glu, Asp, and Cys, and has been used for protein labeling in live cells, were individually incubated with recombinant thrombin. A 300 nM thrombin concentration was chosen in order to mimic its native setting during coagulation, and the reaction mixtures were incubated for four hours in PBS (pH 7.4) at 37° C. The handle transfer efficiency of each aptamer was evaluated by western blot using a streptavidin-horse radish peroxidase (RP) fusion protein and a luminol-peroxide solution for visualization (FIG. 5A). Importantly, a distinct structure-activity relationship was observed, as the electrophile position on the aptamer had a significant impact on its ability to transfer biotin onto thrombin, with position 13 (numbered from 5′ to 3′) appearing to be most efficient. Furthermore, on the protein side, enrichment of biotinylated thrombin via streptavidin pull-down followed by mass spec sequencing revealed that only 5 lysines out of 19 were labeled and that these lysines are located <25 Å of the modified thymidine, based on a co-crystal structure. Notably, K149 and K109-110 were 91% and 74% labeled, respectively, as determined by mass spec sequencing. However, when the same experiment was performed with a shortened, 1 h incubation period, no labeling was detected, indicating that the warhead can exhibit slow handle-transfer kinetics. Nuclease-mediated oligodeoxynucleotide degradation—a limitation to traditional aptamers—occurs on relatively fast (t_{1/2}˜1 h) timescales; we reasoned this issue can be alleviated using the electrophile conjugates if labeling occurred at timescales faster than degradation. Moreover, the overall rate of covalent bond formation is defined by the ratio of the labelling rate constant to the dissociation constant; therefore, for efficient covalent protein labeling it is key to utilize electrophilic warheads with fast reaction rates.

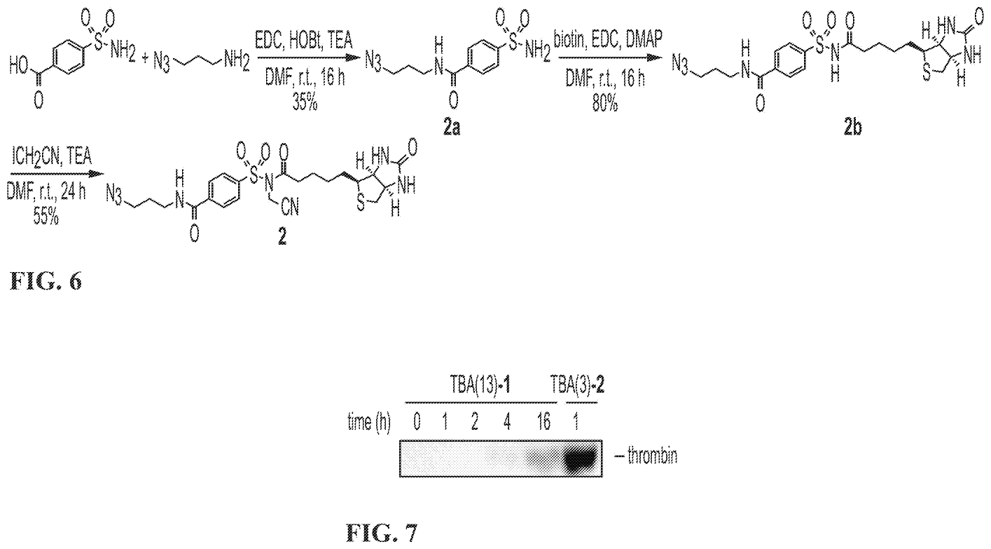

To address this, the higher reactivity N-acyl sulfonamide electrophile 2, which selectively labels lysines, was synthesized in four steps (FIG. 6) and conjugated to TBA. Aptamers modified with 2 were incubated with thrombin for one hour under the same conditions as the previous labeling reaction (FIG. 5B). Like electrophile 1, this warhead showed distinct, position-dependent reactivity, with positions 3 and 7 being the most efficient, while 12 and 13 also showed some labeling. When comparing labeling kinetics of the aptamers TBA(13)-1 and TBA(3)-2, the N-acyl sulfonamide 2 showed significantly faster kinetics than the tosyl 1 and was employed in all further experiments (FIG. 7).

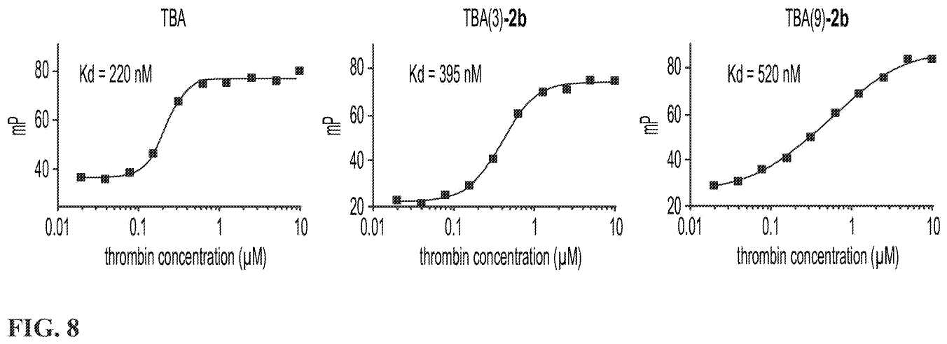

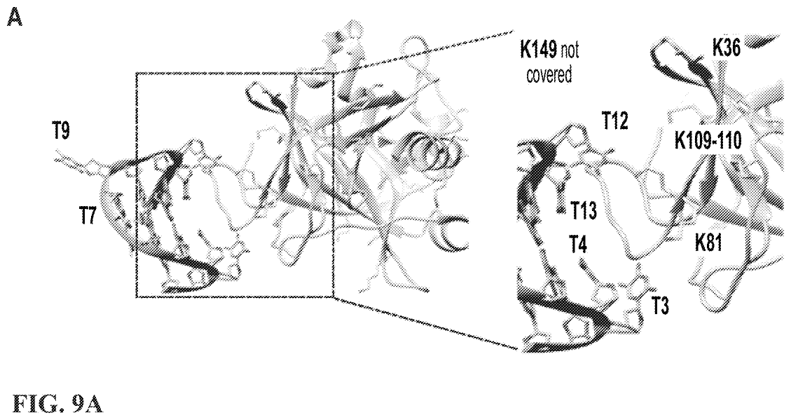

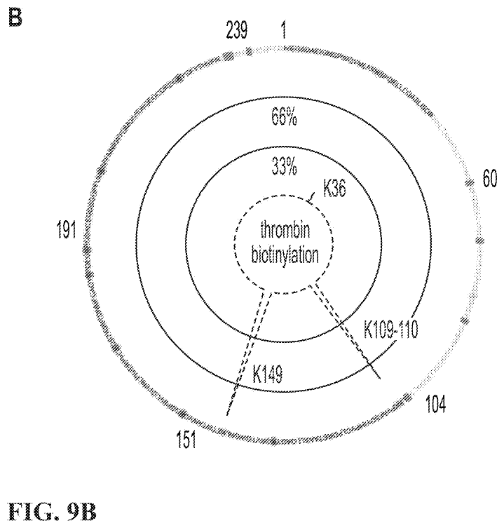

Next, to ensure that the installed electrophile did not reduce the aptamer's binding affinity—thereby impeding overall potency—and that the positional preferences observed in FIG. 5B did not stem from steric interference caused by the warhead in certain positions, the binding constants of TBA modified at position three (TBA(3)) and nine (TBA(9)), which exhibited high and low handle-transferring efficiencies, respectively, were measured. Both aptamers were synthesized to contain the inactivated N-acyl sulfonamide warhead 2b (FIG. 3) as well as a 3′ fluorescein. A fluorescence polarization assay (FIG. 8) revealed very similar binding constants for TBA(3)-2b (395 nM) and TBA(9)-2b (520 nM), which also matched the unmodified aptamer (220 nM). This confirms that the differences in labeling efficiencies were not due to interference with the aptamer-protein interaction (e.g., due to steric hinderance), but are likely due to distinct positioning of the warhead in relation of accessible Lys residues on the protein surface. This is further supported by the fact that TBA(3)-2 only biotinylated 5 out of 19 lysines when incubated with thrombin, as determined by mass spec sequencing. Not surprisingly, these lysines are located at the aptamer-protein binding interface, based on a TBA-thrombin crystal structure (FIG. 9A). Quantification revealed that K149, K109-110, and K36 were 91%, 74%, and 4% biotinylated, respectively, thus demonstrating some site-selectivity of the handle-transferring aptamers (FIG. 9B). The peptide containing K81 was detected only when biotinylated and therefore labeling efficiency cannot be quantified. K109 and K110 are grouped because they appear on the same peptide after tryptic digestion and hence cannot be differentiated. There is also a small part of the protein that we were unable to cover by mass spec sequencing, despite repeated attempts even in the case of unmodified thrombin.

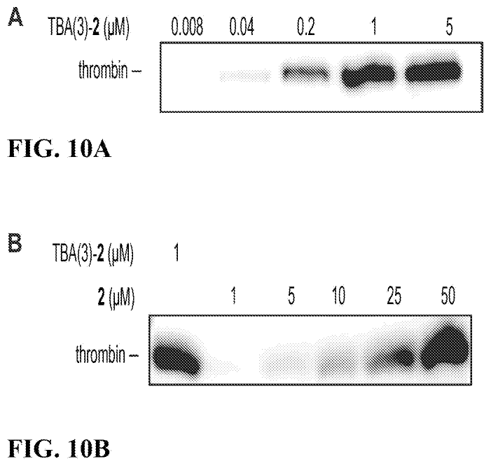

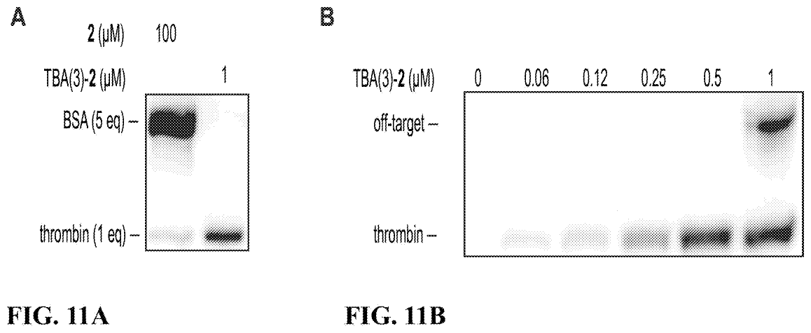

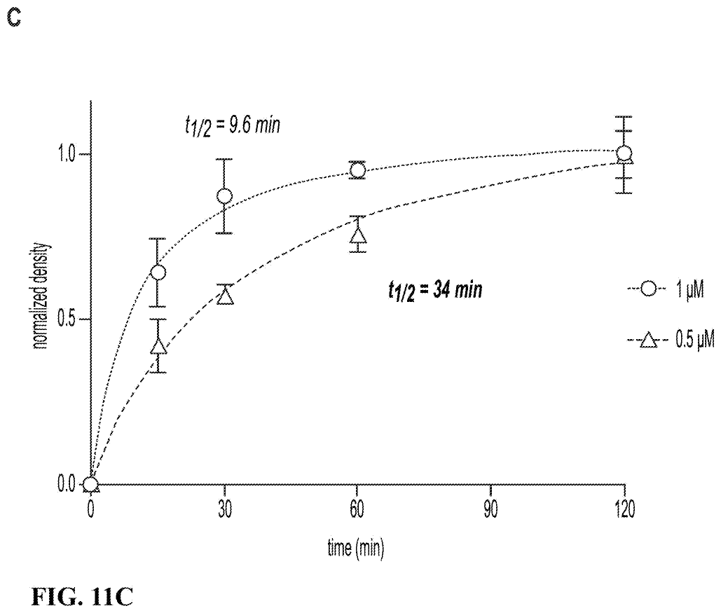

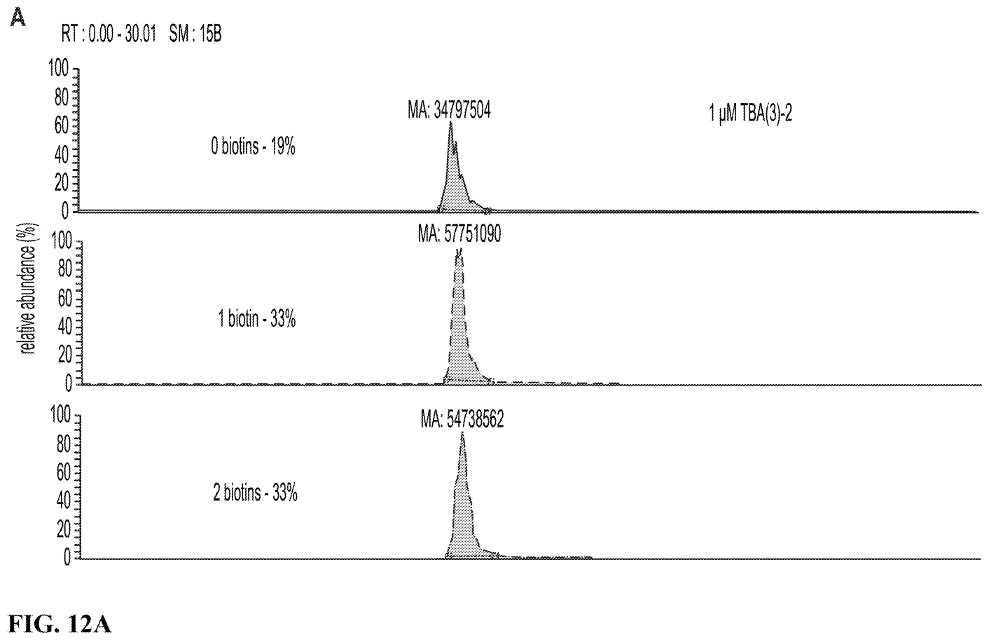

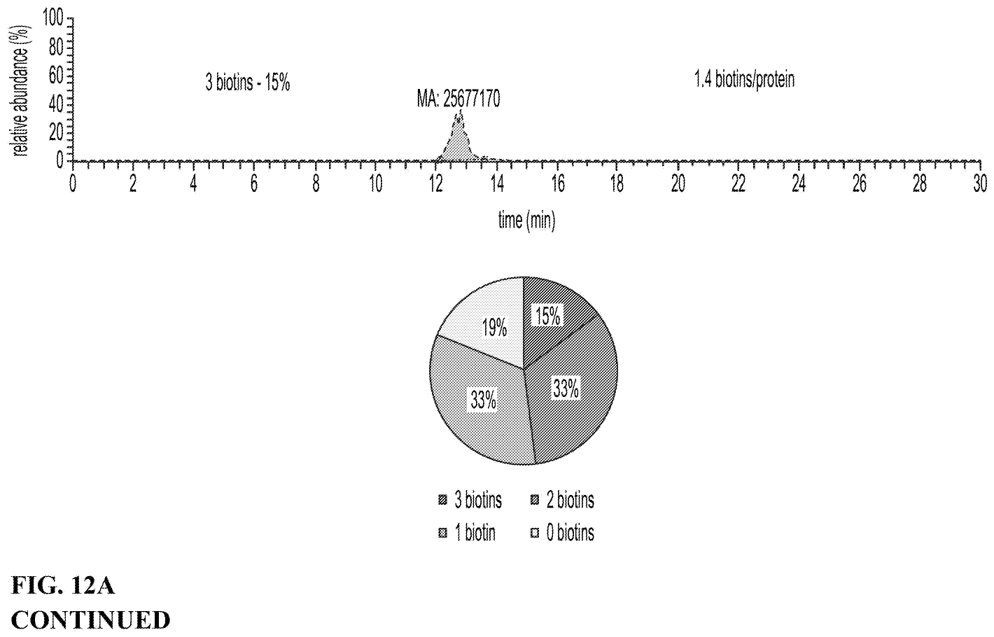

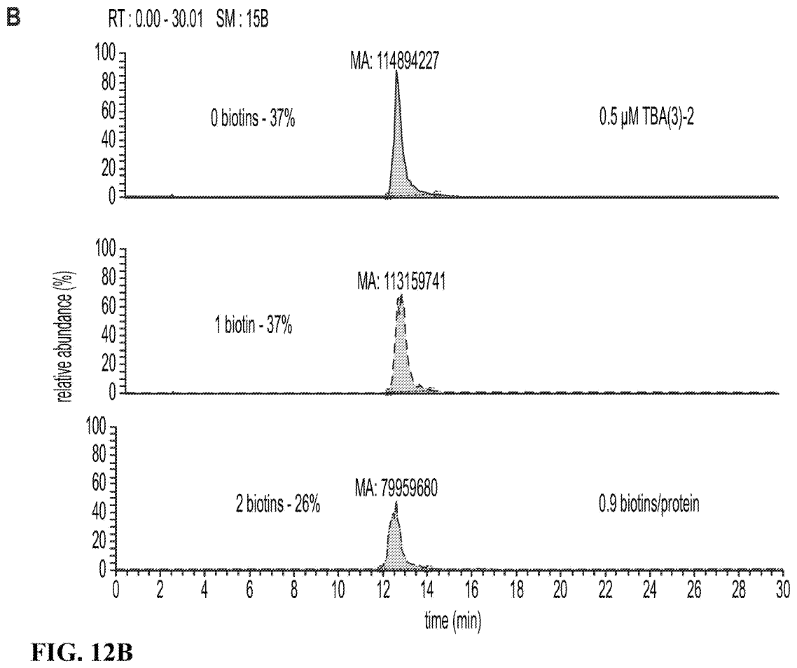

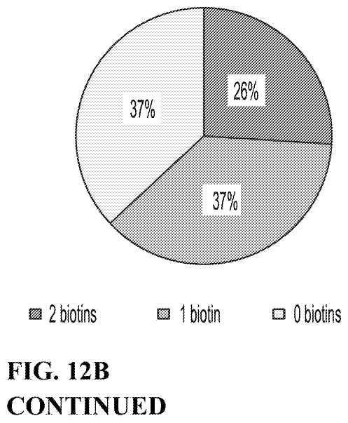

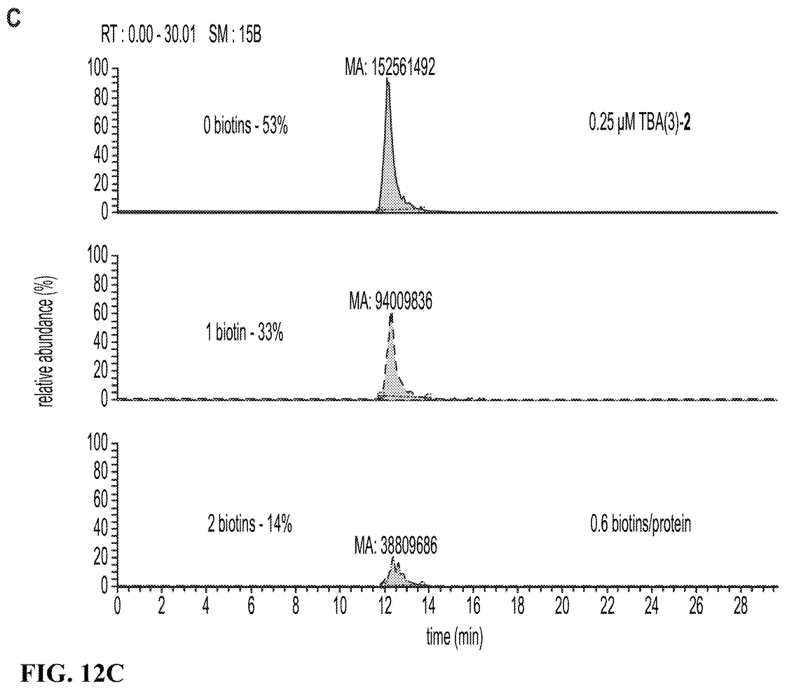

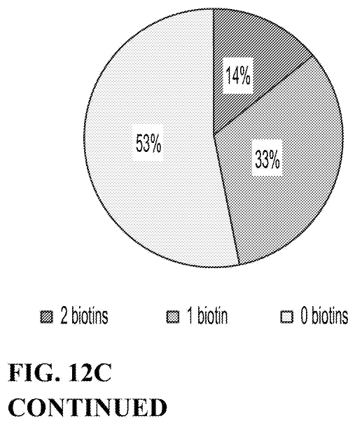

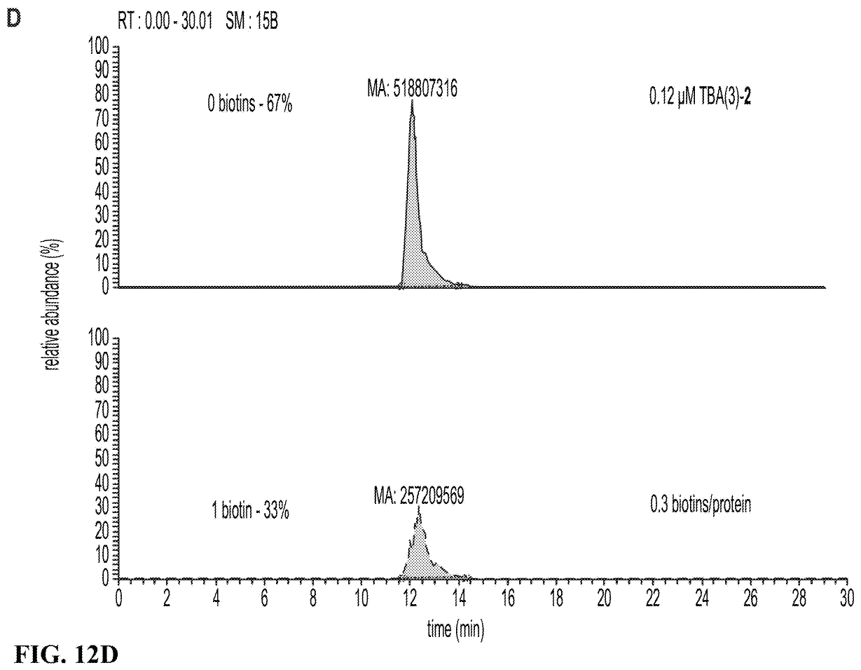

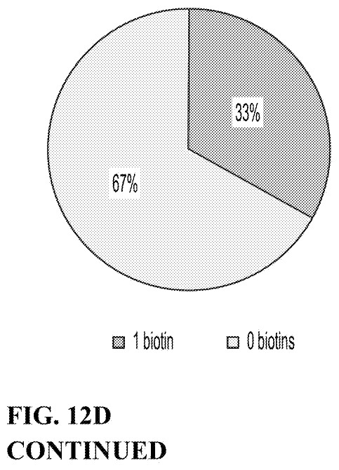

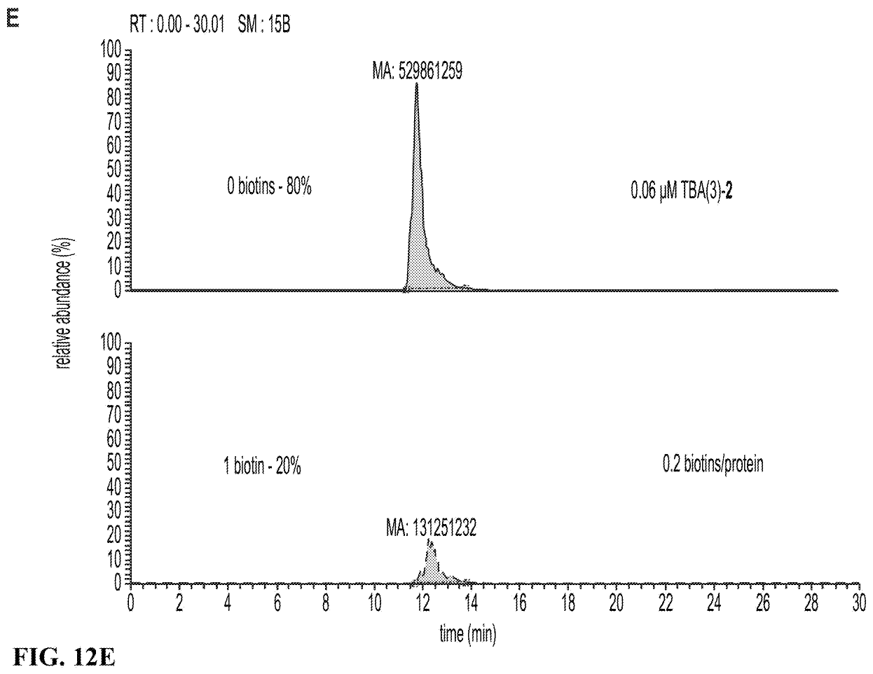

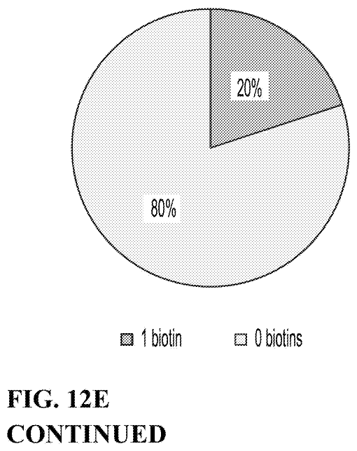

Using the newly established TBA(3)-2, we determined the maximal protein labeling that can be achieved. Thrombin was treated with increasing concentrations of TBA(3)-2 and biotinylation was first observed at 40 nM and reached a maximum at 1 μM (FIG. 10A). Enhancement of labeling kinetics through aptamer-based electrophile presentation to proximal nucleophilic residues was then confirmed by comparing labeling at 1 μM of TBA(3)-2 with that of the unconjugated small molecule electrophile 2 at increasing concentrations. The small molecule alone required a 50-fold higher concentration than the aptamer for similar labeling yields (FIG. 10B). Thus, the proximity effect imparted by the aptamer provided significant enhancements in labeling kinetics, efficiency, and residue specificity. Next, we investigated protein target selectivity of the covalent aptamers and found that thrombin was exclusively biotinylated in the presence of an excess BSA (FIG. 11A), a protein that has three times the number of lysines (60). Additionally, to assess the potential use of the handle-transferring aptamers in thrombin's native environment, thrombin and TBA(3)-2 were incubated in human plasma (˜1.5 mM total protein concentration), and again highly efficient target labeling was observed while no other proteins were biotinylated at aptamer concentrations of up to 500 nM (FIG. 11B). A time-course experiment then revealed that covalent modification of thrombin is not only selective but also fast, with a t/2 of 9.6 min and 34 min at 1 and 0.5 μM of TBA(3)-2, respectively (FIG. 11C). Mass spectrometry analysis showed that 82% of thrombin was modified within 30 min at 37° C. with an average of 1.5 biotins per protein in the presence of 1 μM of TBA(3)-2 (FIG. 12).

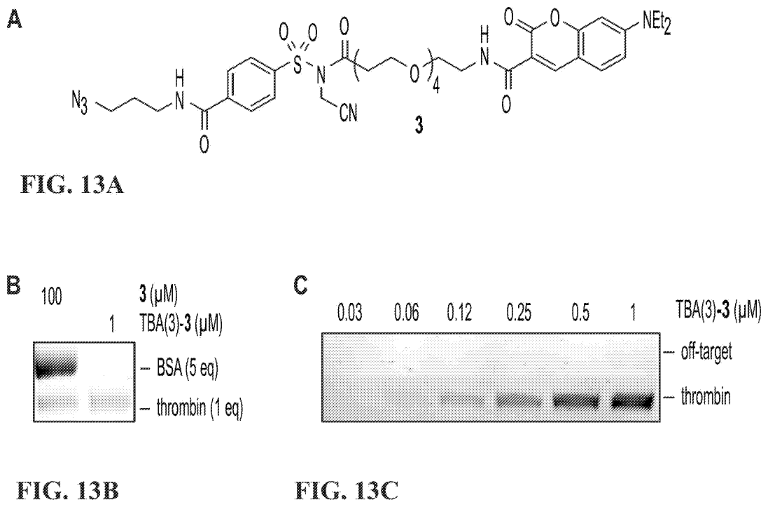

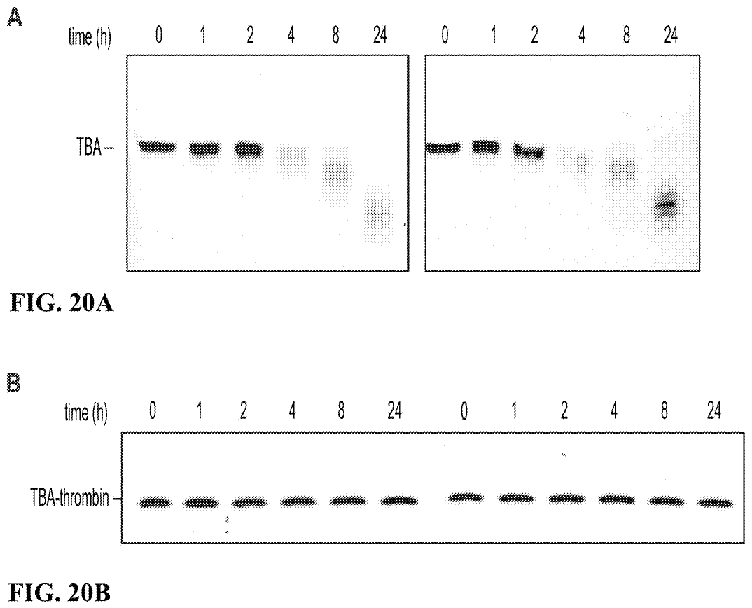

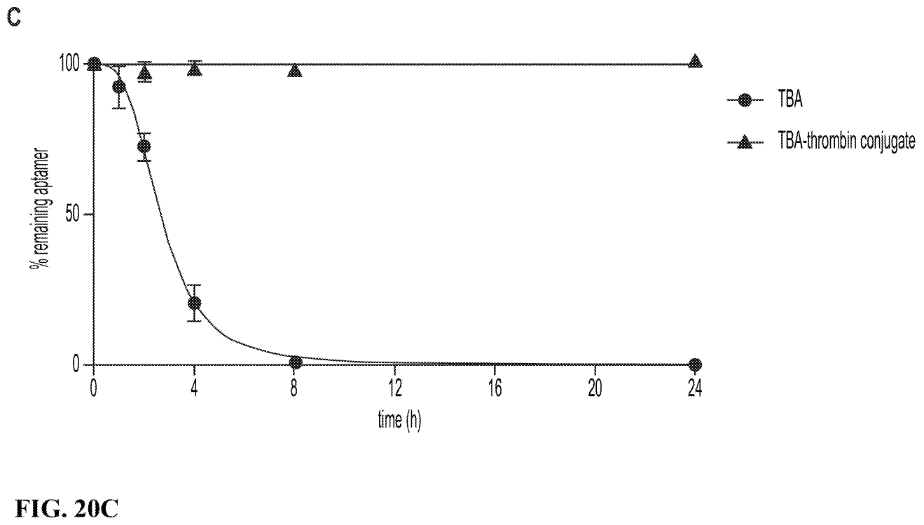

To demonstrate versatility in the delivery of different handles and to simplify detection of labeled proteins, a diethylamino coumarin (DEACM) transferring electrophile 3 was synthesized and then conjugated to T3 of TBA (FIGS. 13A and 20A-20C). The fluorescent conjugate (1 μM) was then used in a thrombin (300 nM) labeling reaction (FIG. 13B), and DEACM was selectively transferred to thrombin, even in the presence of excess BSA (1.5 μM). Furthermore, the covalent aptamer was again selective for the target protein when tested in human plasma and the labeled thrombin was readily visualized through gel-fluorescence imaging (FIG. 13C).

While all covalent labeling reactions so far resulted in release of the aptamer ligand from the protein target, we realized that “inverting” the warhead can result in a covalent crosslink between the targeted protein and the aptamer, rather than a transfer label (FIG. 15A). This was achieved by conjugating the nucleic acid with the electrophile via its amide motif, rather than the aryl sulfonamide as before. Covalent protein-oligonucleotide conjugates have found diverse applications including protein immobilization, assay development, and drug delivery. Furthermore, owing to fast labeling timescales, it was reasoned that the protein can shield the crosslinked aptamer from nuclease-mediated degradation. Few examples exist in which nucleic acids were engineered to covalently bind proteins, including DNA decoys, nucleic acids that the consensus binding site of transcription factors, and aptamers crosslinking to purified and cell-surface proteins.

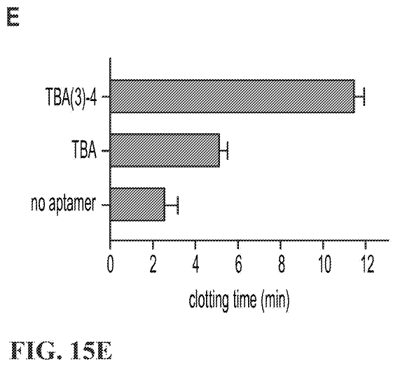

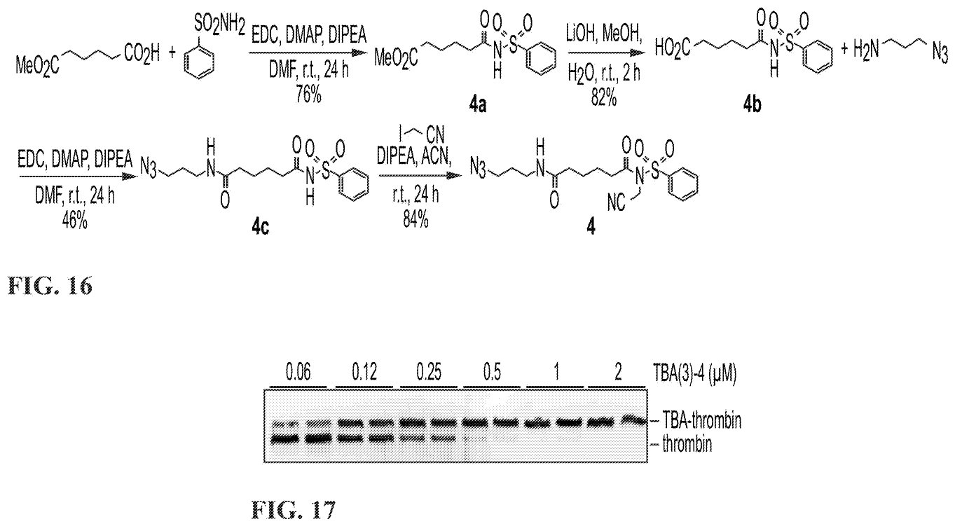

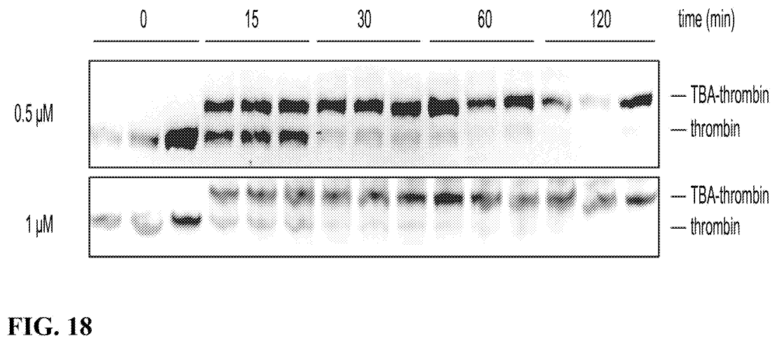

The inverted electrophile 4 was synthesized in 3 steps (FIG. 16) and conjugated to TBA to form TBA(3)-4. It was then incubated at increasing concentrations with 300 nM of thrombin in PBS at 37° C. for 1 h and analyzed via SDS-PAGE. As expected, the aptamer crosslinked to its target, as indicated by a band shift, and the bands were integrated to quantify conjugation yield (FIGS. 15B and 17). Interestingly, only a single aptamer crosslinked per protein, in contrast to the multiple biotin transfers seen before, likely due to the crosslinked aptamer blocking further target interaction with other aptamers. To examine its selectivity, a 5′ fluorescein-modified TBA(3)-4 was generated and incubated in human plasma spiked with thrombin. A denaturing gel (FIG. 15C) again revealed selective on-target crosslinking at aptamer concentrations up to 500 nM (1 h at 37° C.), matching results with the biotin-delivery aptamer (FIG. 11B). A time-course experiment revealed that thrombin crosslinking was nearly quantitative within 1 h (FIGS. 15D and 18). However, while biotinylating and crosslinking kinetics at 1 μM using TBA(3)-2 and TBA(3)-4 were comparable (t_{1/2}=9.6 and 13.6 min respectively), at 500 nM TBA(3)-4 crosslinked to thrombin was significantly faster (t_{1/2}=13.6 min) than biotinylation by TBA(3)-2 (t_{1/2}=34 min). These differences in kinetics can be attributed to competition for thrombin binding that arises between TBA(3)-2 and the electrophile-free aptamer generated and released after biotin transfer. At 1 μM, competition is negligible due to aptamer excess; at 500 nM aptamer and 300 nM protein target, however, competition decreases the labeling kinetics. Competition is not an issue for TBA(3)-4 since once crosslinked, the aptamer is not released and cannot compete for additional binding sites.

Once fast and selective crosslinking to thrombin was confirmed, we sought to determine whether TBA-thrombin complex stabilization via the generated covalent bond can enhance the aptamer inhibition of thrombin's function. Thrombin is a key enzyme in maintaining hemostasis and hyperactivation leads to thrombosis—one of the most common causes of death. To assess enzymatic activity, we capitalized on thrombin's cleavage of fibrinogen into insoluble fibrin, which can be quantified via absorbance at 350 nm. Thrombin was incubated with TBA for 1 h in PBS (pH 7.4) at 37° C. Fibrinogen was added and absorbance was recorded over 30 min (FIG. 19), followed by extrapolation of clotting times (FIG. 15E). Compared to unmodified TBA, TBA(3)-4 lead to a 2-fold prolonged clotting time, demonstrating that the potency of an aptamer can be enhanced through covalent crosslinking.

Because TBA(3)-4 was capable of crosslinking to thrombin in spite of nucleases present in human plasma, we next explored the aptamer's stability once crosslinked. Although nuclease-mediated aptamer degradation has been partially addressed by modification of the 2′ position on the individual nucleotides, tedious structure-activity relationship studies are required to identify positions that can be modified without significantly reducing binding affinity. Fluorescein-modified TBA(3)-4 (1 μM) was incubated with thrombin (300 nM) for 1 h in PBS (pH 7.4) at 37° C. The crosslinking reaction was quenched through addition of glycine (10 mM) followed by a 1 h incubation period. The conjugate was then diluted 10-fold into human plasma and continued to incubate at 37° C. When analyzed by SDS-PAGE, no band shift and no reduction in fluorescence was observed after 24 h, indicating that the crosslinked aptamer is fully stable in human plasma (FIG. 20).

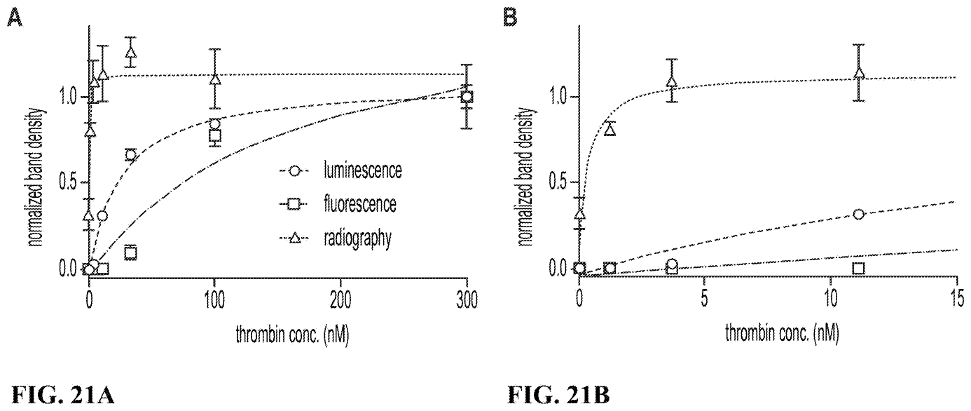

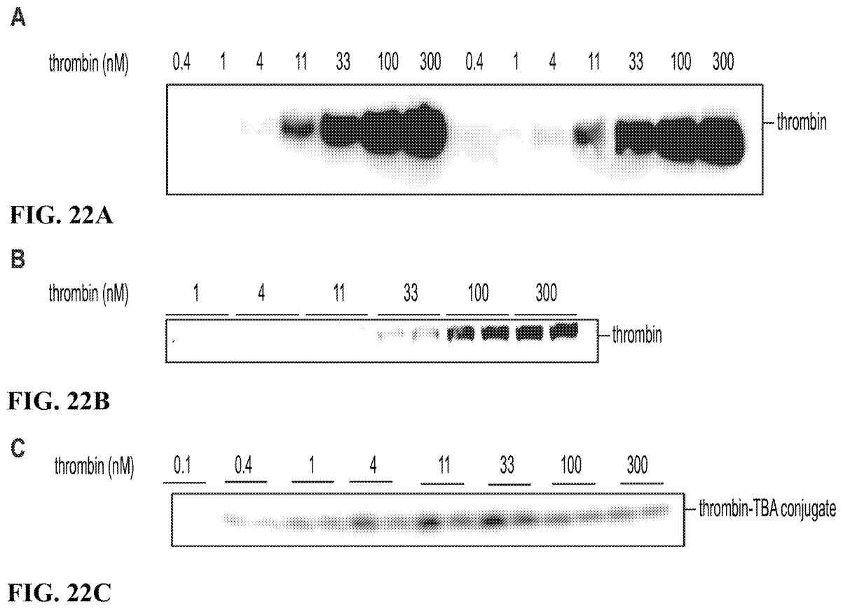

With electrophiles 2, 3, and 4, conjugating biotin, coumarin, and a nucleic acid molecule to thrombin established, we evaluated the diagnostic potential of covalent aptamers by measuring the protein detection limit of the target protein using three approaches. Sensitive thrombin detection has therapeutic implications as concentrations are highly varied between individuals, indicating that thrombotic disorder treatments can be personalized based off these concentrations. Thus, we incubated the biotinylating electrophile 2 with decreasing thrombin concentrations in PBS (pH 7.4) at 37° C. for 1 h. The detection limit was 4 nM when measured via western blot (FIGS. 21 and 22). Compared to traditional western blots, this approach is more economical since oligonucleotides, as opposed to antibodies, can be quickly generated via chemical synthesis, are homogenous, have little batch-to-batch variability, and exhibit long shelf-lives. Additionally, membrane incubation periods are reduced to 1 h due to the high affinity biotin-streptavidin interaction, enabling a faster workflow. To simplify this assay, the detection limit was measured using the fluorophore transferring electrophile 3. The detection limit was 33 nM; however, the same assay has lower limits when used with an electrophile that transfers a brighter fluorophore. Transfer of different fluorophores also lends itself easily to multiplexing, as this approach offers a fast (˜2 h) and convenient method for native protein detection. In an attempt to decrease the detection limit, TBA modified with the crosslinking electrophile 4 was phosphorylated at the 5′ end using a T4 polynucleotide kinase in the presence of γ-³²P ATP. The radiolabeled conjugate was used at a greatly reduced concentration (20 nM) and was incubated with thrombin at decreasing concentrations under the same conditions as before. Radiolabeling of thrombin was analyzed on a denaturing gel that was visualized with a phosphorimager. Using this approach, the detection limit was reduced to 400 fM, thus demonstrating that covalent aptamers provide a platform to detect and quantify proteins of low abundance.

In summary, an aptamer capable of fast and selective protein covalent labeling was developed; it transferred functional motifs such as biotins and fluorophores to native thrombin in buffer as well as in human plasma. Labeling occurs with complete protein specificity as well as good site-selectivity, as only lysines at the aptamer binding interface are modified. Inversion of the crosslinking electrophilic warhead enabled rapid protein-oligonucleotide conjugate formation, with only a single aptamer transferred per protein. The covalent aptamer inhibited thrombin's enzymatic function with greater efficacy than its noncovalent counterpart. Additionally, labeling occurs on timescales that outcompete degradation by endogenous nucleases, and crosslinked aptamers remain immune to nuclease-mediated degradation for >24 h. Each of the three crosslinked chemical motifs enabled a unique approach to protein detection, at concentrations in the femto- to nanomolar range, using very simple western blot, fluorescence, and radiography protocols.

### b) Methods

### (1) Chemical Synthesis

### 5-Trimethylsilylethynyl-2′-deoxyuridine (S1a

Pd(PPh₃)₄ (320 mg, 0.282 mmol), 5-iodo-2′-deoxyuridine (1 g, 2.82 mmol), CuI (130 mg, 0.68 mmol), TEA (1.1 mL, 7.9 mmol), and trimethylsilylacetlene (2 mL, 14.1 mmol) were dissolved in DMF (35 mL) and stirred for 16 h at room temperature. The reaction mixture was concentrated in vacuo and the residue was purified by flash chromatography on silica gel, eluting with MeOH/DCM (3:47) to give Sla (910 mg, 99%) as a brown solid. NMR and HRMS data matched those previously reported.

### 5-Ethynyl-2′-deoxyuridine (SIb)

TBAF dissolved in THF (4.22 mL, 4.22 mmol) was added to a solution of compound S1a (910 mg, 2.81 mmol) in THE (10 mL). The reaction mixture was stirred at room temperature for 1 h. Volaties were evaporated in vacuo and the residue was purified by flash chromatography on silica gel, eluting with MeOH/DCM (1:9) to give Sib (520 mg, 73%) as a yellow solid. NMR and HRMS data matched those previously reported.

### 2′-Deoxy-5′-DMTr-5-ethynyluridine (S1c)

DMTrCl (805 mg, 2.38 mmol) was added to a solution of compound S1b (500 mg, 1.98 mmol) in pyridine (10 mL). The reaction mixture was stirred for 16 h at room temperature and then quenched with MeOH (1 mL). The reaction mixture was concentrated in vacuo and the residue was purified by flash chromatography on silica gel, eluting with EtOAc/hexanes (7:13) to give S1c (830 mg, 76%) as a white solid. NMR and HRMS data matched those previously reported.

### 2′-Deoxy-5′-DMTr-5-ethynyluridine-3′-cyanoethylphosphoramidite (S1)

Ethylthio-1H-tetrazole (8.4 mL of 0.25 M solution in ACN, 2.08 mmol) was added to a solution of compound 1c (580 mg, 1.04 mmol) and 2-cyanoethyl N,N,N′,N′-tetraisopropylphosphorodiamidite (0.65 mL, 2.08 mmol) in ACN (20 mL). The reaction mixture was stirred at room temperature for 3 h. The reaction mixture was diluted with DCM (80 mL) and washed with sat. NaHCO₃ (1×20 mL). The aqueous phase was extracted with DCM (3×40 mL). The combined organic layers were dried over Na₂SO₄ (1 g), filtered, and concentrated in vacuo. The residue was purified by flash chromatography on silica gel, eluting with EtOAc/hexanes (9:11) to give 1 (638 mg, 81%) as a white solid. NMR and HRMS data matched those previously reported.

### 6-Azidohexan-1-amine (1a)

Thionyl chloride (3.2 mL, 44 mmol) was added to a solution of 6-amino-1-hexanol (1.29 mL, 10 mmol) in toluene (10 mL). The reaction mixture was heated to 78° C. for 1 h and then concentrated in vacuo. The remaining residue and sodium azide (1.95 g, 3 mmol) were dissolved in water (10 mL). The reaction mixture was then stirred for 2 h at 80° C. It was then cooled to 0° C. and basified with potassium hydroxide (500 mg) to pH 12. The aqueous solution was then extracted with DCM (3×30 mL) and the combined organic layers were dried over Na₂SO₄ (1 g), filtered, and concentrated in vacuo to give compound 1a (754 mg, 62%) as a clear oil. NMR and HRMS data matched those previously reported.

### 3-((6-Azidohexyl)carbamoyl)benzenesulfonyl chloride (1b)

compound 1a (744 mg, 5.3 mmol) and TEA (2.96 mL, 21 mmol) were dissolved in DCM (15 mL). In a separate flask, 3-(chlorosulfonyl)benzoyl chloride (2 mL, 13.2 mmol) was dissolved in DCM (7.4 mL) and the solution was cooled to 0° C. The solution containing compound 1a was then added dropwise. The reaction mixture was stirred for 30 min at 0° C. and then an additional 1.5 h at r.t. The reaction mixture was concentrated in vacuo and the residue was purified by flash chromatography on silica gel, eluting with DCM to give compound 1b (541 mg, 30%) as a yellow oil. ¹H NMR (500 MHz, CDCl₃) δ 8.13 (s, 1H), 7.96 (d, 1H, J=8), 7.92 (d, 1H, J=8), 7.57 (t, 1H, J=8) 3.18 (t, 2H, J=7), 1.53 (m, 2H), 1.30 (m, 6H). ¹³C NMR (500 MHz, CDCl₃) δ 164.9, 144.7, 136.5, 133.9, 130.3, 129.4, 125.0, 51.3, 40.4, 29.5, 28.7, 26.5, 26.4. HRMS (ESI⁻) calcd for C₁₂H₁₅ ClN₄O₃S (M−H)⁻ 329.0553, found 329.0556.

### 2,2-Dimethyl-4-oxo-3,8,11-trioxa-5-azatridecan-13-yl3-((6-azidohexyl)carbamoyl)benzenesulfonate (1c)

compound 1b (541 mg, 1.57 mmol) then N-Boc-PEG2-alcohol (183 μL, 0.78 mmol) were dissolved in DCM (4.9 mL). DIPEA (403 μL, 2.35 mmol) and DMAP (29 mg, 0.24 mmol) were added and the reaction mixture was stirred at r.t. for 6 h. The reaction mixture was concentrated in vacuo and the residue was purified by flash chromatography on silica gel, eluting with MeOH/DCM (1:49) to give compound 1c (72 mg, 16%) as a yellow oil. ¹H NMR (500 MHz, CDCl₃) δ 8.20 (s, 1H), 7.81 (d, 1H, J=7), 7.98 (d, 1H, J=12), 7.57 (t, 1H, J=8), 6.20 (s, 1H), 3.65-3.45 (m, 10H), 3.18 (t, 2H, J=7), 2.91 (quart, 2H, J=7), 1.53-1.48 (m, 13H), 1.39-1.35 (m, 6H). ¹³C NMR (500 MHz, CDCl₃) δ 173.4, 165.4, 136.4, 135.8, 133.6, 130.2, 129.6, 126.2, 70.6, 70.0, 69.9, 69.8, 68.5, 61.8, 60.1, 59.5, 55.5, 51.4, 40.6, 40.2, 39.2, 38.2, 35.9, 31.9, 31.2, 29.7, 29.3, 28.8, 28.1, 28.0, 26.6, 26.4, 25.7, 22.7. HRMS (ESI⁻) calcd for C₂₄H₃₉N₅O₈S (M−H)⁻ 556.2436, found 556.2453.

### 2-(2-(2-(5-((3aS,4S,6aR)-2-Oxohexahydro-1H-thieno[3,4-d]imidazol-4-yl)pentanamido)ethoxy)ethoxy) ethyl3-((6-azidohexyl)carbamoyl)benzenesulfonate (1)

compound 1c (15 mg, 0.03 mmol) was dissolved in a DCM/TFA (4:3, 3.5 mL) mixture and stirred for 2 h at r.t. The reaction mixture was concentrated in vacuo and remaining solvent was co-evaporated with toluene (1 mL) three times. The remaining residue was dissolved in DMF (4 mL) then biotin-NHS (10 mg, 0.03 mmol) and DIPEA (16 μL, 0.09 mmol) were added to the solution and the reaction mixture was stirred for 2 h at r.t. The reaction mixture was concentrated in vacuo and the residue was purified by flash chromatography on silica gel, eluting with MeOH/DCM (3:47) to give compound 1 (4.5 mg, 24%) as a white solid. ¹H NMR (500 MHz, CDCl₃) δ 8.31 (s, 1H), 8.15 (d, 1H, J=7), 7.95 (d, 1H, J=12), 7.60 (t, 1H, J=8), 6.51 (s, 1H), 5.92 (s, 1H), 4.43 (m, 1H), 4.25 (m, 1H), 4.15 (m, 2H), 3.63 (m, 3H), 3.51-3.45 (m, 6H), 3.40-3.34 (m, 4H), 3.22 (t, 2H, J=6.5), 3.06-3.02 (m, 2H), 2.86-2.80 (m 1H), 2.14 (t, 2H J=7), 1.62-1.50 (m, 8H), 1.39-1.31 (m, 8H). ¹³C NMR (500 MHz, CDCl₃) δ 173.4, 165.4, 163.5, 136.4, 135.8, 133.6, 130.2, 129.6, 126.2, 70.6, 70.0, 69.9, 69.8, 68.5, 61.8, 60.1, 59.5, 55.5, 51.4, 40.6, 40.2, 39.2, 38.2, 37.3, 35.9, 35.5, 31.9, 31.2, 29.7, 29.3, 28.8, 28.1, 28.0, 26.6, 26.4, 25.6, 24.9, 22.7. FIRMS (ESI+) calcd for C₂₉H₄₅N₇O₈S₂ (M+H)⁺684.2844, found 684.2837.

### 5-Iodo-5′-DMTr-2′-deoxyuridine (S2a)

Starting material 5-iodo-2′-deoxyuridine (1 g, 2.82 mmol) was dissolved in pyridine (20 mL). DMTrCl (1 g, 2.96 mmol) was added to the solution and the reaction mixture was stirred for 16 h at r.t. The reaction was quenched with 2 mL MeOH and the resulting solution was stirred an additional 15 min. The reaction mixture was concentrated in vacuo and the residue was purified by flash chromatography on silica gel, eluting with EtOAc/hexanes (7:3) to give compound S2a (1.5 g, 81%) as a white solid. NMR and HRMS data matched those previously reported.

### 5-(Triisopropylsilyl)ethynyl-5′-DMTr-2′-deoxyuridine (S2b)

Compound S2a (1.5 g, 2.28 mmol) was dissolved in DMF (30 mL). Tetrakis(triphenylphosphine)palladium (266 mg, 0.23 mmol) and copper(I) iodide (88 mg, 0.46 mmol) were then added followed by TEA (953 μL, 6.84 mmol) and (triisopropylsilyl)acetylene (2.5 mL, 11.4 mmol). The reaction mixture was allowed to stir for 16 h at r.t. It was then concentrated in vacuo and the residue was purified by flash chromatography on silica gel, eluting with EtOAc/hexanes (6:4) to give compound S2b (1.1 g, 68%) as a white solid. NMR and HRMS data matched those previously reported.

### 5-(Triisopropylsilyl)ethynyl-5′-DMTr-2′-deoxyuridine-3′-cyanoethyl phosphoramidite (S2)

Compound S2b (100 mg, 0.14 mmol) was dissolved in acetonitrile (3 mL) followed by 2-Cyanoethyl N,N,N′,N′-tetraisopropylphosphorodiamidite (67 μL, 0.21 mmol) and 5-(ethylthio)-1H-tetrazole (27 mg, 0.21 mmol). The reaction mixture was stirred for 4 h at r.t. It was then diluted with DCM (50 mL) and washed with sat. NaHCO₃ (1×10 mL). The aqueous layer was then reextracted with DCM (3×40 mL). The combined organic layer was dried over Na₂SO₄ (1 g), filtered, and concentrated in vacuo. The residue was purified by flash chromatography on silica gel, eluting with EtOAc/hexanes (3:7) to give 2b (84 mg, 66%) as a white solid. NMR and HRMS data matched those previously reported.

### N-(3-Azidopropyl)-4-sulfamoylbenzamide (2a)

EDC (2.3 g, 12 mmol), TEA (3.4 mL, 24 mmol), 4-sulfamoylbenzamide (1.6 g, 8 mmol), HOBt (1.6 g, 12 mmol), and 3-azido-1-propylamine (800 mg, 8 mmol) were dissolved in DMF (32 mL). The reaction mixture was stirred at room temperature for 16 h. It was then diluted with EtOAc (200 mL) and washed with sat. NaHCO₃ (1×300 mL), 5% citric acid (1×300 mL), and brine (1×300 mL). The organic layer was dried over Na₂SO₄ (1 g), filtered, and concentrated in vacuo. The residue was purified by flash chromatography on silica gel, eluting with MeOH/DCM (3:47) to give 2a (793 mg, 35%) as a white solid. ¹H NMR (500 MHz, d6-DMSO) δ 8.70 (t, 1H, J=5.5), 7.98 (d, 2H, J=8.5), 7.90 (d, 2H, J=8.5), 7.48 (s, 1H), 3.44 (t, 2H, J=6.5), 3.36 (t, 2H, J=6.5), 1.79 (quin, 2H, J=6.5). ¹³C NMR (500 MHz, d6-DMSO) δ 165.8, 146.7, 137.9, 128.3, 126.1, 49.0, 37.3, 28.8. HRMS (ESI⁻) calcd for C₂₀H₂₇N₇O₅S₂ (M−H)⁻ 282.0655, found 282.0661.

### N-(3-Azidopropyl)-4-(N-(5-((4S)-2-oxohexahydro-1H-thieno[3,4-d]imidazol-4-yl)pentanoyl)sulfamoyl)benzamide (2b)

Compound 2a (100 mg, 0.35 mmol), biotin (130 mg, 0.53 mmol), TEA (150 μL, 1.1 mmol), EDC (205 m-g, 1.1 mmol), and DMAP (10 mg, 0.07 mmol) were dissolved in DMF (5 mL). The reaction mixture was stirred for 16 h at room temperature. The reaction mixture was concentrated in vacuo and the residue was purified by flash chromatography on silica gel, eluting with AcOH/MeOH/DCM (1:5:94-1:10:89) to give 2b (143 mg, 80%) as a white solid. ¹H NMR (500 MHz, CD₃OD) δ 8.08 (d, 2H, J=8.5), 7.99 (d, 2H, J=8.5), 4.47-4.45 (m, 1H), 4.22-4.20 (m, 1H), 4.49 (t, 2H, J=7), 3.43 (t, 2H, J=7), 3.23-3.18 (m, 1H), 3.10-3.08 (m, 1H), 2.91-2.88 (m, 1H), 2.69 (d, 1H, J=13), 2.26 (t, 2H, J=7), 1.91-1.86 (m, 2H), 1.66-1.45 (m, 4H), 1.32-1.21 (m, 4H). ¹³C NMR (500 MHz, CD₃OD) δ 172.5, 167.2, 164.7, 163.3, 142.0, 139.1, 128.0, 127.5, 61.9, 60.2, 55.4, 48.8, 46.5, 39.6, 37.2, 35.3, 28.3, 28.3, 27.9, 24.1, 7.82. HRMS (ESI⁺) calcd for C₂₀H₂₇N₇O₅S₂ (M+H)⁺ 510.1587, found 510.1576.

### N-(3-Azidopropyl)-4-(N-(cyanomethyl)-N—(S-((4S)-2-oxohexahydro-1H-thieno[3,4-d]imidazol-4-yl)pentanoyl)sulfamoyl)benzamide (2)

Compound 2b (17 mg, 33 μmol), TEA (23 μL, 165 μmol), and iodoacetonitrile (24 μL, 330 μmol) were dissolved in DMF (250 μL). The reaction mixture was stirred for 24 h at room temperature. The reaction mixture was concentrated in vacuo and the residue was purified by flash chromatography on silica gel, eluting with MeOH/DCM (1:49-3:47) to give 2 (10 mg, 55%) as a white solid. ¹H NMR (500 MHz, CD₃OD) δ 8.15 (d, 2H, J=9), 8.10 (d, 2H, J=9), 4.92 (s, 2H), 4.50-4.47 (m, 1H), 4.27-4.24 (m, 1H), 3.50 (t, 2H, J=7), 3.44 (t, 2H, J=7), 3.15-3.12 (m, 1H), 2.93-2.90 (m, 1H), 2.73-2.47 (m, 3H), 1.92 (quin, 2H, J=7), 1.78-1.55 (m, 4H), 1.50-1.41 (m, 2H), 1.25-1.36 (m, 4H). ¹³C NMR (500 MHz, CD₃OD) δ 172.1, 166.7, 164.7, 140.9, 140.0, 128.3, 127.9, 115.3, 61.9, 60.2, 55.5, 48.8, 39.6, 37.3, 35.3, 33.1, 28.3, 28.0, 27.9, 23.9. HRMS (ESI⁺) calcd for C₂₂H₂₉N₈O₅S₂ (M+H)⁺549.1696, found 549.1674.

### tert-Butyl 3-(2-(7-(diethylamino)-2-oxochromane-3-carboxamido)ethoxy)propanoate (3a)

EDC (18 mg, 92 μmol), TEA (32 μL, 229 μmol), HOBt (13 mg, 92 μmol), and amino-PEG₄-t-butyl ester (25 mg, 76 μmol) were added to a solution of 7-(Diethylamino)coumarin-3-carboxylic acid (20 mg, 76 μmol) dissolved in DMF (500 μL). The reaction mixture was stirred for 16 h at room temperature and then concentrated in vacuo. The residue was purified by flash chromatography on silica gel, eluting with EtOAc/hexanes (17:3) to give 5a (40 mg, 93%) as a yellow oil. ¹H NMR (500 MHz, CDCl₃) δ 9.04 (s, 1H), 8.63 (s, 1H), 7.35 (m, 1H), 6.61 (d, 1H, J=6.5), 6.45 (s, 1H), 3.69 (m, 18H), 3.43 (quart, 4H, J=7), 2.47 (t, 2H, J=6.5), 1.42 (s, 9H), 1.21 (t, 6H, J=7). ¹³C NMR (500 MHz, CDCl₃) δ 170.9, 163.49, 162.5, 157.7, 152.6, 148.1, 131.2, 126.7, 125.9, 117.6, 110.9, 110.1, 109.9, 108.3, 96.6, 80.5, 70.7, 70.6, 70.6, 70.5, 70.5, 70.3, 69.8, 66.9, 45.1, 39.6, 36.3, 28.1, 12.4. HRMS (ESI⁺) calcd for C₂₉H₄₅N₂O₉ (M+H)⁺ 565.3119, found 565.3106.

### tert-Butyl 1-(7-(diethylamino)-2-oxo-2H-chromen-3-yl)-1-oxo-5,8,11,14-tetraoxa-2-azaheptadecan-17-oate (3b)

A solution of compound 5a (40 mg, 71 μmol) in DCM (0.5 mL) and TFA (0.5 mL) was stirred at room temperature for 1 h. The reaction mixture was concentrated in vacuo. The residue was suspended in toluene (1 mL) and concentrated in vacuo three times and dissolved in DMF (1 mL). TEA (30 μL, 212 μmol), EDC (40 mg, 212 μmol), DMAP (spatula tip), and compound 2a (20 mg, 71 μmol) were added to the solution and the reaction mixture was stirred for 16 h at room temperature. It was then concentrated in vacuo and the residue was purified by flash chromatography on silica gel, eluting with MeOH/DCM (1:19) to give 5b (30 mg, 55%) as a yellow oil. ¹H NMR (500 MHz, CD₃OD) δ 8.49 (s, 1H), 7.98 (d, 4H, J=8.5), 7.42 (d, 1H, J=9), 6.69 (d, 1H, J=9), 6.43 (s, 1H), 3.56 (m, 22H), 3.33 (m, 4H), 2.37 (t, 2H, J=5.5), 1.82 (m, 4H), 1.12 (t, 6H, J=7.5). ¹³C NMR (500 MHz, CD₃OD) δ 170.6, 167.5, 167.1, 157.7, 153.2, 147.9, 146.2, 141.9, 139.0, 137.6, 131.3, 128.0, 127.9, 127.6, 127.5, 127.4, 125.9, 110.3, 108.1, 95.9, 78.1, 77.9, 77.6, 70.2, 70.2, 70.1, 70.0, 70.0, 69.2, 65.8, 56.4, 48.8, 44.7, 39.2, 37.2, 36.5, 28.3, 11.4. HRMS (ESI⁺) calcd for C₃₅H₄₈N₇O₁₁S (M+H)⁺774.3127, found 774.3105.

### N-(2-((4-((3-Azidopropyl)carbamoyl)phenyl)sulfonyl)-1-cyano-3-oxo-6,9,12,15-tetraoxa-2-azaheptadecan-17-yl)-7-(diethylamino)-2-oxo-2H-chromene-3-carboxamide (3)

A solution of compound 5b (30 mg, 39 μmol), TEA (27 μL, 194 μmol), and iodoacetonitrile (56 μL, 776 μmol) in DMF (2 mL) was stirred for 1 h at 45° C. The reaction mixture was concentrated in vacuo and the residue was purified by flash chromatography on silica gel, eluting with MeOH/DCM (1:24) to give 5 (7 mg, 22%) as a yellow solid. ¹H NMR (500 MHz, CD₃OD) δ 9.11 (s, 1H), 8.65 (s, 1H), 8.51 (s, 1H), 8.03 (d, 2H, J=8.5), 7.96 (d, 2H, J=8.5), 7.90 (m, 4H), 7.45 (d, 1H, J=9), 6.73 (s, 1H, J=14), 6.48 (s, 1H), 4.80 (s, 2H), 3.30-3.62 (m, 24H), 2.89 (t, 2H, J=6), 1.82 (m, 4H), 1.13 (t, 6H, J=7). ¹³C NM/R (500 MHz, CD₃OD) δ 170.6, 167.5, 166.7, 164.0, 162.6, 157.8, 153.2, 147.9, 146.3, 140.7, 139.9, 137.6, 131.2, 128.2, 127.9, 127.6, 127.0, 126.5, 125.9, 115.2, 110.3, 108.8, 108.1, 95.9, 78.1, 77.9, 77.6, 70.3, 70.2, 70.2, 70.1, 70.1, 70.0, 69.2, 65.8, 48.8, 44.6, 39.2, 37.3, 37.2, 36.3, 33.2, 28.3, 28.3, 11.1. HRMS (ESI⁺) calcd for C₃₇H₄₉N₈O₁₁S (M+H)⁺ 813.3236, found 813.3201.

### Methyl 6-oxo(phenylsulfonamido)hexanoate (4a)

Benzenesulfonamide (518 mg, 3.3 mmol), monomethyl adipate (481 mg, 3.0 mmol), DMAP (439 mg, 3.6 mmol), and DIEA (1.9 mL, 10.8 mmol) were dissolved in DMF (15 mL). EDC (690 mg, 3.6 mmol) was added, and the reaction mixture was stirred at r.t. for 24 h. The reaction mixture was concentrated in vacuo. The residue was dissolved with EtOAc (100 mL), washed with 1M HCl (1×15 mL), H₂O (1×15 mL), and brine (1×5 mL), dried over Na₂SO₄ (1 g), and concentrated in vacuo. The product was purified by flash chromatography MeOH/DCM (1:20) to give compound 4a (756 mg, 76%) as a white solid. NMR and HRMS data matched those previously reported.

### 6-Oxo(phenylsulfonamido)hexanoic acid (4b)

LiOH (88 mg, 3.5 mmol) was added to a solution of 4a (210 mg, 0.70 mmol) in MeOH (2.9 mL) and H₂O (0.7 mL) at r.t. and the reaction mixture was stirred for 2 h. The reaction was quenched with 1 M HCl (5 mL) and the product was extracted with EtOAc (1×20 mL). The organic layer was washed with H₂O (1×5 mL) and brine (1×5 mL), dried over Na₂SO₄ (1 g), and concentrated in vacuo to give compound 4b (163 mg, 82%) as a white solid. NMR and HRMS data matched those previously reported.

### N′-(3-Azidopropyl)-N⁶-(phenylsulfonyl)adipamide (4c)

compound 4b (100 mg, 0.35 mmol) was added to a solution of 2 (59 mg, 0.29 mmol) in dry DMF (14 mL) followed by EDC (54 mg, 0.35 mmol), DMAP (43 mg, 0.35 mmol) and DIPEA (0.30 mL, 1.68 mmol). The reaction mixture was stirred at r.t. for 24 h and then concentrated in vacuo. The residue was purified by flash chromatography MeOH/DCM (3:7-1:1) to give compound 4c (49 mg, 46%) as a white solid.

### N¹-(3Aazidopropyl)-N-(cyanomethyl)-N-(phenylsulfonyl)adipamide (4)

DIPEA (0.2 mL, 1.12 mmol) and iodoacetonitrile (40 uL, 0.56 mmol) were added to a solution of 4c (50 mg, 0.14 mmol) in dry DMF (3.4 mL). The reaction mixture was stirred at room temperature for 24 h in the dark. The residue was dissolved with EtOAc (20 mL), washed with brine (3×10 mL), dried with Na₂SO₄ (1 g), and then the solvent was evaporated in vacuo. The residue was purified by flash chromatography (MeOH:DCM-0→5% MeOH) to yield compound 8 (47 mg, 84%) as a white solid. ¹H NMR (400 MHz, CD₃OD) δ 8.07 (d, 2H, J=7.6), 7.83 (t, 1H, J=7.6), 7.73 (t, 2H, J=7.6), 4.88 (s, 2H), 3.25 (quart, 2H, J=5.6), 2.76 (t, 2H, J=6), 2.17 (t, 2H, J=6.8), 1.79 (quin, 2H, J=6.8), 1.57 (m, 4H). ¹³C NMR (400 MHz, CD₃OD) δ 174.4, 172.0, 163.5, 138.6, 134.5, 129.5, 127.5, 115.3, 36.3, 35.5, 35.2, 32.9, 30.2, 28.3, 24.7, 23.7. HRMS (ESI⁺) calcd for C₁₇H₂₃N₆O4S (M+H)⁺ 407.1496, found 407.1478.

### (2) General Protocol of Oligonucleotide Ethanol Precipitation

Ethanol precipitation was achieved through addition of sodium acetate (3 M, pH 5.2, 1/10 volume) to an oligonucleotide solution (10-500 μM, 1 volume) in nuclease free water followed by 100% ethanol (3 volumes). The resulting solution was vortexed and cooled in a −80° C. freezer for 1 h. Oligonucleotides were pelletted using a centrifuge (>16,000 g, 4° C., 10 min). The supernatant was then discarded, the pellet was washed with 70% ethanol (3 volumes), and pelleted once again using the same conditions. The supernatant was then removed and the remaining pellet was dried using a speedvac (ThermoFisher) for 5 min.

### (3) Oligonucleotide Synthesis and Purification

Oligonucleotide syntheses were conducted using an Expedite 8909 DNA/RNA Synthesizer (Distribio, NY, USA) employing standard β-cyanoethyl phosphoramidite chemistry. Oligonucleotides were synthesized on a 200 nanomole scale using 500 Å derivatized CPG solid phase supports obtained from Glen Research (VA, USA) using a 5′-DMTr-OFF approach. Reagents and phosphoramidites for automated DNA synthesis were also obtained from Glen Research. Synthesis cycles with coupling times of 2 and 10 mins were employed for unmodified and modified phosphoramidites, respectively, at 0.07 M concentration. Coupling efficiency in each cycle was monitored by following the release of dimethoxytrityl (DMTr) cations after each deprotection step. No significant loss of DMTr was noted following the addition of any of the modified phosphoramidites to the oligonucleotide. Following synthesis, cleavage from support and deprotection of the oligonucleotides were carried out with 1 mL of concentrated NH₄OH solution at room temperature for 16 h.

Purification was performed on 20% native PAGE (140 V, 1.25 h). The gel was visualized via UV shadowing and the major band was excised from the gel. It was then passively diffused into 1 mL nuclease free water (4 h, 37° C.). The supernatant was collected and concentrated in vacuo using a speedvac. Residues were ethanol precipitated to remove remaining salts and then dissolved in 100 μL nuclease free water to achieve ca. 100 μM stock solution. The concentrations of purified oligonucleotides were determined by a NanoDrop Spectrophotometer (ND1000).

DNA synthesis reagent and phosphoramidite list:
dA-CE phosphoramidite (10-1000)
Ac-dC-CE phosphoramidite (10-1015)
dmf-dG-CE phosphoramidite (10-1029)
dT-CE phosphoramidite (10-1030)
tetrahydrofuran/2,6-lutidine/acetic anhydride (40-4010)
5-ethylthio-1H-tetrazole (ETT) (30-3040)
0.02 M iodine in tetrahydrofuran/pyridine/water (88:10:2) (40-4032)
3% dichloroacetic acid in dichloromethane (40-4040)
acetonitrile, anhydrous (40-4050)

### (4) TIPS Deprotection General Protocol

Oligonucleotides modified with TIPS-EdU using phosphoramidite S2 were suspended in DMF (0.4 mL). TBAF (0.1 mL, 1 M in THF) was added and the resulting solution was incubated at 45° C. for 30 min with vigorous shaking. The reaction was quenched with TEAA (Glen Research, 2 M, pH 7). Oligonucleotides were precipitated with ethanol and redissolved in 200 μL of water. They were then purified by HPLC, injecting 100 μL at a time using a gradient of 5-30% buffer B over 30 min at a flow rate of 1 mL/min on a Waters XBridge Oligonucleotide BEH C18 column (4.6 mm×50 mm, 130 Å, 2.5 μm). Buffers used were 0.1 M TEAA as buffer A and ACN as buffer B and the column oven was set to 40° C.

### (5) Alkyne-Bearing Oligonucleotide Post-Synthesis Modification

Alkyne-bearing oligonucleotides were modified using an optimized [3+2]cycloaddition protocol. In a 1.7 mL centrifuge tube triethylammonium acetate buffer (5 μL, 2 M, pH 7) was added to 50 μL of the alkyne-bearing oligonucleotide (20-200 μM) in nuclease-free water. DMSO (55 μL) was added and the solution was vortexed. A solution of the azide (5 μL, 10 mM) in DMSO was added and the reaction mixture was vortexed. An ascorbic acid solution (12 μL, 5 mM) was added, and the reaction mixture was vortexed again. Argon was bubbled through the reaction mixture for 30 seconds and copper(II)-tris[(1-benzyl-1H-1,2,3-triazol-4-yl)methyl]amine (TBTA) in 55% DMSO (6.5 μL, 10 mM), prepared by dissolving copper (II) sulfate pentahydrate (25 mg) in deionized water (5 mL), TBTA (58 mg) in deionized water (5.5 mL), and combining the two solutions, was quickly added. After briefly vortexing, the reaction mixture was incubated at room temperature for 2 h. Oligonucleotides were isolated through ethanol precipitation. Products were analyzed via HPLC on a Waters XBridge Oligonucleotide BEH C18 column (4.6 mm×50 mm, 130 Å, 2.5 μm). Buffers used were 0.1 M TEAA as buffer A and ACN as buffer B and the column oven was set to 40° C. The injected sample (5 μL) was then analyzed using a gradient of 5-30% buffer B over 30 min at a flow rate of 1 mL/min. Since conjugation proceeded to completion, products were not purified further.

### (6) General Protocol for Aptamer-Mediated Handle-Transfer and Crosslinking

Thrombin (Haemotologic Technologies) or BSA (Thermo Fisher) were diluted to 3 μM or 15 μM, respectively, in PBS pH 7.4. The thrombin solution (2 μL) was further diluted with PBS (16 μL) or human plasma (16 μL, Millipore Sigma P9523) doped with yeast tRNA (G-Biosciences 786058, 0.1 mg/mL) as a competitor for nonspecific protein-oligonucleotide interactions. If performing a selectivity experiment, thrombin was diluted with PBS (14 μL) instead and the BSA solution (2 μL) was added. The electrophilic aptamer was diluted to 3 μM in PBS and then added (2 μL) to the thrombin solution. It was then incubated at 37° C. for the indicated amount of time. Total reaction volume was 20 μL.

### (7) Laemmli Loading Buffer (6×) Recipe

Tris buffer (1 M, pH 6.8, 15 mL), SDS (6 g), Glycerol (30 mL), 2-mercaptoethanol (15 mL), and bromophenol blue (0.1 g) were combined in a 50 mL conical tube. Deionized water was added to 50 mL and the solution was vortexed until the solids dissolved. The loading buffer was aliquoted and stored at −20° C.

### (8) Analysis of Biotinylated Proteins

After aptamer-mediated biotin transfer, samples (20 μL) were boiled in Laemmli sample buffer (4 μL) and separated by 10% (v/v) SDS-PAGE gel electrophoresis (140 V, 1.25 h). Afterwards, proteins were transferred (80 V, 1.5 h) to a PVDF membrane (GE Healthcare) and the membrane was blocked in blocking buffer (5% BSA in 1×TBS with 0.1% [v/v] Tween 20) for 1 h at room temperature. The blots were probed with streptavidin-HRP (1:10000 dilution, Thermo Fisher N100) in 1×TBS with 0.1% [v/v] Tween 20 (10 mL) at room temperature for 1 h with rocking. Chemiluminescence was developed using SuperSignal West Pico Chemiluminescent Substrate (Thermo Fisher, 5 mL) and imaged on a ChemiDoc Imaging System (BioRad) using automated exposure settings. Bands were integrated using ImageJ by drawing rectangles around the lanes and analyzing using the plot lanes function. Band integrations were normalized to the highest intensity band. Errors represent standard deviations from the mean.

### (9) Analysis of Coumarin-Modified Proteins

After aptamer-mediated coumarin transfer, samples (20 μL) were boiled in Laemmli sample buffer (4 μL) and separated by 10% (v/v) SDS-PAGE gel electrophoresis (140 V, 1.25 h). Afterwards, the gel was imaged on a ChemiDoc Imaging System (BioRad) using automated exposure settings. Bands were integrated using ImageJ by drawing rectangles around the lanes and analyzing using the plot lanes function. Band integrations were normalized to the highest intensity band. Errors represent standard deviations from the mean.

### (10) Analysis of Aptamer-Crosslinked Protein Through Staining

After aptamer-protein crosslinking, samples (20 μL) were boiled in Laemmli sample buffer (4 μL) and separated by 10% (v/v) SDS-PAGE gel electrophoresis (140 V, 1.25 h). Afterwards, the gel was developed using a silver stain (ThermoFisher 24612) and imaged on a ChemiDoc Imaging System (BioRad) using automated exposure settings. Bands were integrated using ImageJ by drawing rectangles around the lanes and analyzing using the plot lanes function. Band integrations were normalized to the highest intensity band. Errors represent standard deviations from the mean.

### (11) Analysis of Aptamer-Crosslinked Protein Through Radiography

For ³²P-labelling, aptamer (4 μL, 1 μM) was mixed with 10× T4 PNK buffer (NEB, 2 μL), gamma ³²P-ATP (PerkinElmer, 3000 Ci/mmol, 1 μL), T4 PNK (NEB, 1 μL), and nuclease-free water (12 μL), and incubated at 37° C. for 1 hr. The isotope labelled dsDNAs were purified using Microspin™ G-25 Columns (GE Healthcare) and used for gel shift assays, assuming 100% recovery from the G-25 column. Conjugation to protein target was carried out as described in the general protocol using 20 nM as the final concentration of the aptamer. Afterwards, samples (20 μL) were boiled in Laemmli sample buffer (40 μL) and separated by 10% (v/v) SDS-PAGE gel electrophoresis (140 V, 1.25 h). Then the gels were exposed to a phosphor screen for 2 h and scanned by Typhoon FLA7000 IP Phosphorimager (GE Healthcare). Bands were integrated using ImageJ by drawing rectangles around the lanes and analyzing using the plot lanes function. Band integrations were normalized to the highest intensity band. Errors represent standard deviations from the mean.

### (12) Biotinylated Thrombin Pull-Down and Analysis by MS

Pull-down and elution was performed using a modified protocol. An aptamer-mediated biotinylation of thrombin using the general protocol was performed (20 μL total volume). Protein biotinylation was quenched through addition of glycine (2 μL, 100 mM) and boiling at 95° C. for 5 min. The solution was then diluted with PBS (178 VL, pH 7.4). Streptavidin sepharose resin (Cytiva 17511301, 5 μL), washed 2× with buffer A (NP-40 (1 mL), SDS (0.1 g) in PBS (100 mL total volume), 0.5 mL) and two times with PBS (0.5 mL), was added to the solution and incubated for 2 h at 4° C. with gentle agitation. The resin was washed 3× with buffer A, 2× with buffer B (NaCl (23.4 g), NP-40 (1 mL), SDS (0.1 g) in PBS (100 mL total volume), 0.5 mL), and once with 50 mM Tris-HCl (pH 8, 0.5 mL). The resin was pelleted (100 g, 30 sec) after each incubation or wash step. The resin was then resuspended in elution buffer (0.2 g SDS, biotin (73 mg) in PBS (100 mL total volume), 50 μL) and boiled at 95° C. for 15 min. Samples were allowed to cool, diluted with Laemmli buffer (10 μL), and analyzed by 10% (v/v) SDS-PAGE gel electrophoresis (140 V, 1.25 h). The gel was developed with a silver stain (ThermoFisher 24612). The band was cut from the gel and submitted to the University of Pittsburgh Biomedical Mass Spectrometry Center for protein sequencing.

### (13) Stability in Human Serum

Fluorescein-modified TBA(3)-4 (2 μL, 10 μM) was incubated with or without thrombin (2 μL, 2 μM)) in PBS (16 μL, pH 7.4) for 1 h at 37° C. in 20 μL total volume. Crosslinking was quenched by diluting samples with glycine (2 μL, 100 mM) and incubating an additional 1 h at 37° C. The sample (20 μL) was diluted with human plasma (180 μL). The sample was incubated at 37° C. for the indicated amount of time. Samples (20 μL) were diluted with Laemmli buffer (4 μL) and boiled at 95° C. for 5 min. Conjugate samples (20 μL) were analyzed by 10% (v/v) SDS-PAGE gel electrophoresis (140 V, 1.25 h). Unmodified aptamer samples (20 μL) were diluted with Laemmli buffer (4 μL) and analyzed by 10% (v/v) SDS-PAGE gel electrophoresis (140 V, 1.25 h). The gel was then imaged on a ChemiDoc Imaging System (BioRad) using automated exposure settings. Bands were integrated using ImageJ and normalized to the highest intensity band. Errors represent standard deviations from the mean.

### (14) Thrombin Activity Assay

Thrombin (5 μL, 5 μM) was diluted with PBS (40 μL, pH 7.4) and then the indicated aptamer (5 μL, 5 μM) was added. The solution was incubated for 1 h at 37° C. in duplicate in a 96-well plate. Then, fibrinogen was added to a final concentration of 2 mg/mL and 350 nm absorbance was measured every 2.5 min using a plate reader (Tecan Infinite M1000 Pro). Errors represent standard deviations from the mean.

### (15) Fluorescence Polarization Assay

The indicated 3′-fluorescein-modified aptamer was diluted with Tris-HCl (50 mM, pH 7.5) to 4 nM in a 384-well plate. Thrombin was then serially diluted to 2× the final intended concentration in Tris-HCl (50 mM, pH 7.5) and added to the aptamer solution in a 1:1 ratio, such that the final aptamer concentration was 2 nM and the final volume was 20 μL. The solution was incubated at r.t. for 15 min and fluorescence polarization was analyzed using a plate reader (Tecan Infinite M1000 Pro, 494 nm excitation, 521 nm emission, 200 flashes, 300 ms settle time).

### (16) Time-Course Experiments

Thrombin (2 μL, 3 μM) was diluted with PBS (16 μL, pH 7.4) and TBA(3)-2 (2 μL, 5 μM or 10 μM) was added. Starting times were staggered such that all reactions were simultaneously quenched through addition of glycine (2 μL, 100 mM), diluting with Laemmli loading buffer (4 μL), and boiling samples (20 μL) at 95° C. for 5 min. They were then analyzed by 10% (v/v) SDS-PAGE gel electrophoresis (140 V, 1.25 h). Gels were visualized depending on label according to general protocols.

(17) Oligonucleotide characterization by MALDI

Aptamers (100 μM, 1 μL) were co-spotted on a MALDI plate with matrix (1 μL, 17.8 mg 5-methoxysalicylic acid and 11.3 mg diammonium hydrogen citrate in 1 mL 50% acetonitrile). The spot was dried under a gentle stream of air. The plate was again spotted with matrix (1 μL) and dried. Mass spectra were then collected on a Bruker Ultraflextreme MALDI-MS equipped with a Smartbeam II™ (Nd:YAG) laser in linear-negative ion mode and 10 laser shots were accumulated at a 2000 Hz acquisition rate. The diameter of the acquisition area was restricted to 2000 μm using a random walk path.

### 2. Example 2: Modify the Reaction Conditions, Electrophile, Nucleotide Linkage, and Nucleotide Position to Understand and Optimize Both Protein and Amino Acid Residue Specificities

### a) Improve Site-Specificity Through Reaction Conditions, Electrophile Selection, and Linker Rigidity.

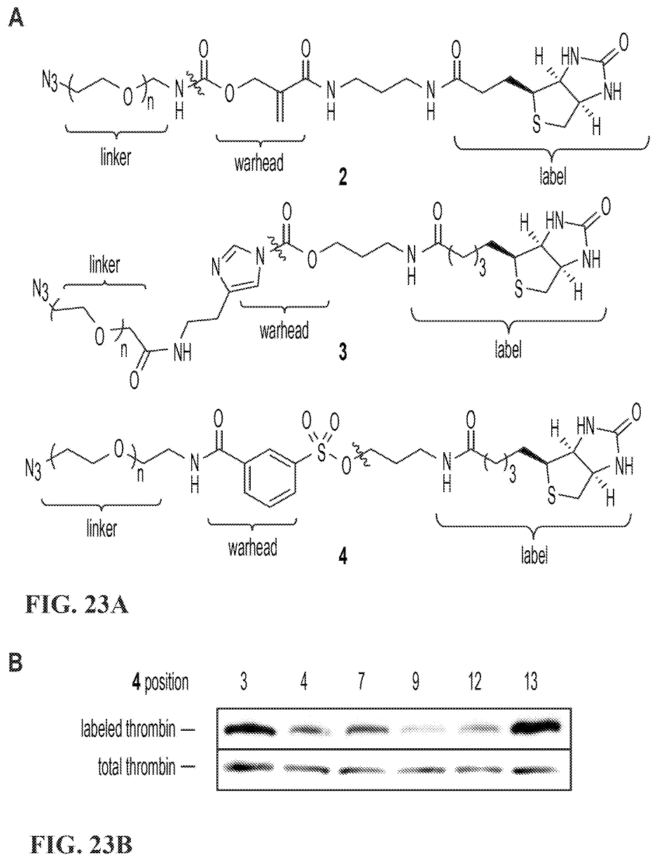

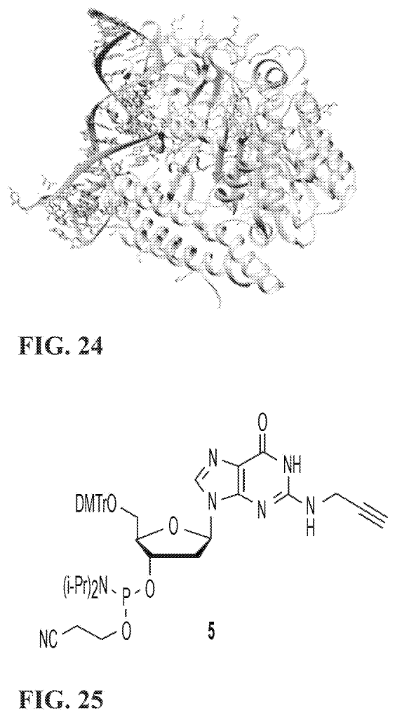

Bioconjugation approaches benefit not only from complete target specificity, but also from selective reactions of certain amino acid residues on the surface of a protein of interest. Although we observed fast labeling kinetics and exquisite protein specificity using N-acyl sulfonamide warheads (see FIG. 9), site-selectivity can still be improved, as 5 lysine residues were labeled. The site-selectivity of the other TBAs can be investigated due to different distances from the warhead position to the nearest lysine:e.g., 19 Å in TBA(7)-1 versus 11 Å in TBA(3)-1. Shorter reaction times (e.g., 5 min instead of 30 min) and lower temperatures (e.g., 4° C. instead of 37° C.) can occur, since labeling of the most proximal residue likely occurs with faster kinetics than others. Furthermore, both hydrophobic (CH₂)ₙ, PEG linkers, and rigid oxime linkers have interactions with the protein which affects warhead positioning. Modification of these parameters improves labeling of fewer/select lysines. By utilizing a novel acrylamide-derived warhead capable of covalent handle transfer exclusively to Cys—a rare residue compared to Lys (FIG. 23A) increasing residue specificity can be achieved. For example, when PDB-6RTI is complexed with PTK7, the aptamer is proximal to lysines and cysteines at its protein binding surface (FIG. 24)

### b) Broaden Target Scope Through Electrophile Selection, Linker Length, and Attachment to Other Nucleotides

Not all applications for covalent aptamers require site-specific labeling. For example, for protein detection, protein degradation, inhibition of protein function, and immune response stimulation, the transfer of multiple handles to different sites at the protein surface may be preferred.

Linker lengths can be increased to allow for more residues to be reached on the protein surface. Alkyne-bearing nucleotides other than thymidine can be generated, particularly guanidine (FIG. 25), to expand the repertoire of positions that the warhead can be installed on. Many aptamers, including TBA, utilize a G-quadruplex structure to bind their targets and thus are abundant in guanidines. The alkyne is installed on 5 such that it does not interfere with G-quadruplex formation, based on hydrogen bonding patterns. Furthermore, to expand upon the targetable residue repertoire, two additional warheads: the N-acyl imidazole 3 and the tosylate 4 can be used. The former has been reported to transfer a label to Lys, Ser, Thr, and Tyr while the latter transfers it to His, Lys, Tyr, Glu, Asp, and Cys. These warheads have been utilized in conjunction with small molecule ligands to deliver handles to intracellular and membrane-bound proteins on live cells. We have already tested the tosylate 4 in conjunction with TBA (FIG. 23B), and observed position-dependent biotinylation of thrombin.

### 3. Example 3: Protein Bioconjugation

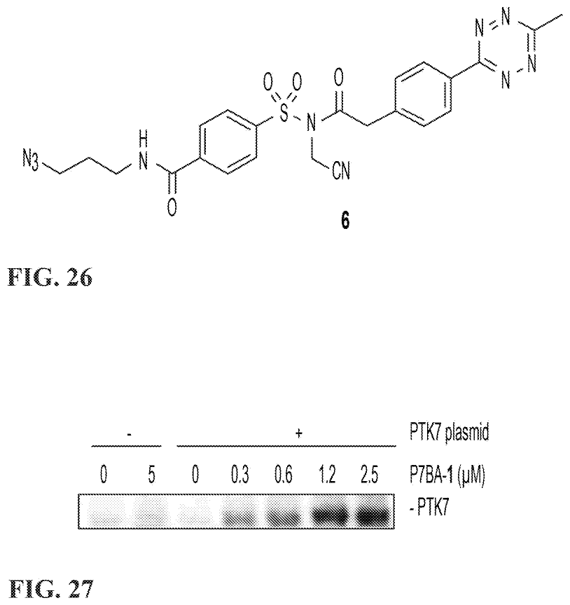

Herein, we have demonstrated successful delivery of biotin and fluorophore motifs. In order to broaden the protein bioconjugation scope of this methodology, while maintaining compatibility with our warhead installation on synthetic aptamers via [3+2]cycloaddition, we can also deliver tetrazine handles. Tetrazines are well established bioconjugation handles that enable rapid, high-yielding, and fully orthogonal ligation of virtually any molecule of interest via a rapid inverse-electron demand Diels-Alder (IEDDA) reaction. The acid-bearing tetrazine can be coupled with a sulfonamide and the resulting N-acylsulfonamide can be alkylated with iodoacetonotrile to generate the tetrazine-delivering warhead 6 (FIG. 26). and successful conjugation with 4 can be confirmed through IEDDA with TCO-rhodamine (Click Chemistry Tools), followed by purification and fluorescence readout, as well as mass spec analysis to identify site-specificity and extent of the bioconjugation reactions. In our experience using unnatural amino acids, IEDDA reactions occur rapidly and quantitatively in the modification of proteins.

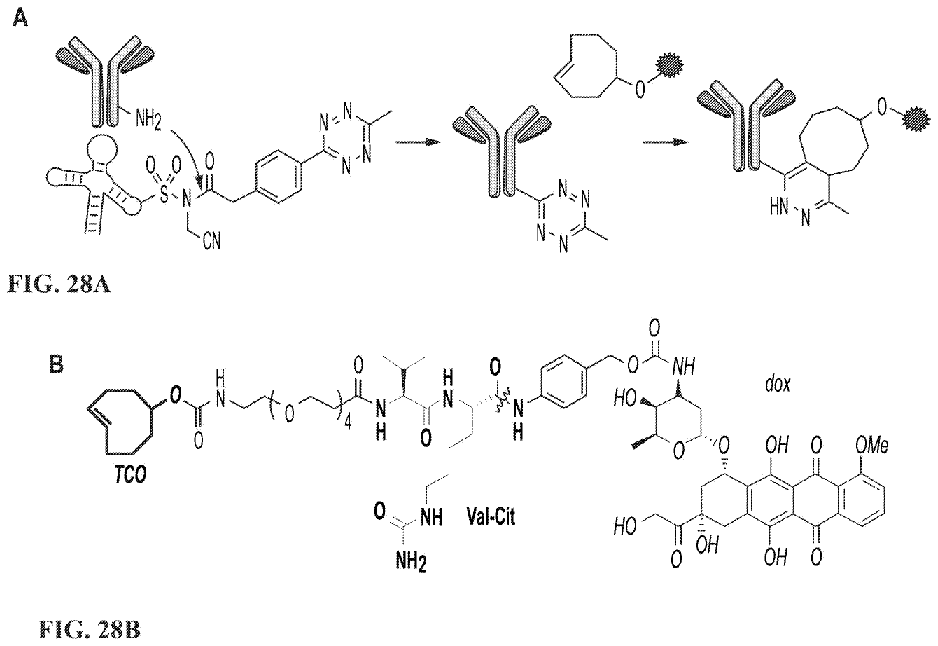

This approach can also be applied to the synthesis of antibody-drug conjugates (ADCs). Comprised of a monoclonal antibody, a cytotoxic payload, and a linker, ADCs hold promise to combine the exquisite target selectivity of immunotherapy with the efficacy of chemotherapy. However, the assembly of ADCs from native antibodies has been plagued by inconsistent payloads, due to heterogenous mixtures resulting from reactions of NHS-esters with lysines. To address this, several approaches (e.g., disulfide re-bridging and chemoenzymatic conjugation) toward homogenous ADCs were developed, each with a unique set of advantages and challenges (e.g., disulfide scrambling, need for glycan removal, limited drug-to-antibody ratio DAR). We can utilize the covalent aptamer approach to generate homogenous ADCs. Covalent aptamers complement current conjugation technologies because 1) they do not require genetic engineering of the antibody, 2) they do not require use of external enzymes, and 3) they are not prone to disulfide scrambling. Furthermore, by adjusting temperatures, reaction times, and aptamer concentrations, or through the selection of new aptamers, we expect to generate covalent aptamers producing different DARs while maintaining complete reproducibility of which lysines are modified. In initial studies, we can utilize the tetrazine-delivering electrophile 6 (FIG. 26) in conjunction with a known antibody-binding aptamer (ABA; Table 1) that targets the constant region of human IgG1 thereby providing a generalizable solution (FIG. 28A). The approach described herien is also generalizable with regard to the cytotoxic payload, due to the robust tetrazine-TCO conjugation chemistry. We can utilize ABA-4 to transfer a tetrazine to rituximab, an FDA approved anti-CD20 antibody, and conjugate to it TCO-modified doxorubicin 7 (FIG. 28B). The position of doxorubicin and the DAR can be determined by mass spec sequencing. The optimal DAR range is 2-4, and handle transfer conditions can be adjusted to achieve DARs of 2, 3, and 4. Following established protocols, we can test ADC toxicity towards CD20⁺ Raji cells while using unmodified Rituximab and CD20⁻ Jurkat cells as negative controls.

The transferred bioconjugation handle allows for additional applications such as protein PEGylation using commercial TCO reagents (Broad-Pharm) for the development of therapeutics, e.g., PEG interferon-α2a via a known aptamer, and protein immobilization for the development of sensors. Warheads that transfer other handles, such as Halotag and SNAPtag ligands for protein-protein assemblies, are readily accessible as well.

### 4. Example 4: Aptamer-Catalyzed Covalent Bond Formation

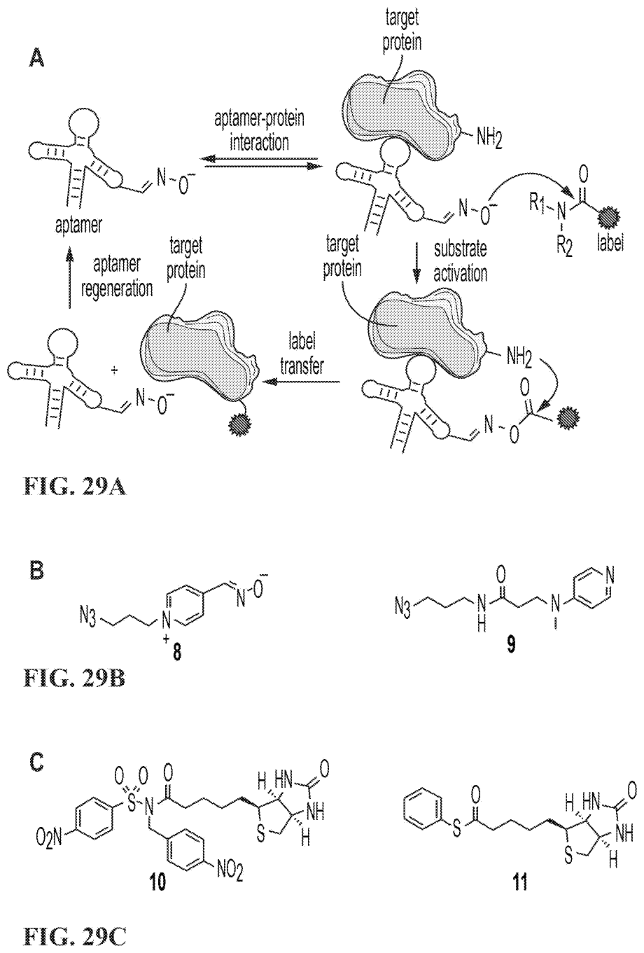

Complementary to aptamer-directed affinity labeling, we propose a electrophile-activating platform for chemically labeling of native proteins (FIG. 29A). This is based on successes with organocatalyst-modified small molecule ligands which activate their substrates only within the vicinity of the target protein. The aptamer-catalyst conjugate functions by selectively binding to the targeted protein, where the catalyst facilitates acyl transfer to a proximal nucleophilic residue on the target protein. To direct acyl transfer, we can conjugate TBA to the oxime 8 and the DMAP analog 9, established organocatalysts that have demonstrated high efficiency in small molecule-mediated electrophile-activating labeling (FIG. 29B). The catalysts can be installed at T and G positions on TBA to determine whether they have positional preferences similar to the label-delivering aptamers (FIG. 11). TBA-7 (0-1 μM) can be incubated with thrombin (300 nM) for up to 3 h at 37° C. along with the known biotin-delivering acyl substrates 10 and 11 (FIG. 29C). The substrates 10 and 11 have been optimized to be activated by catalysts 8 and 9, respectively. Relative labeling efficiencies, residue specificity, and catalyst turnover numbers can be determined.

### Example 5: Aptamer-Catalyzed Protein Labeling

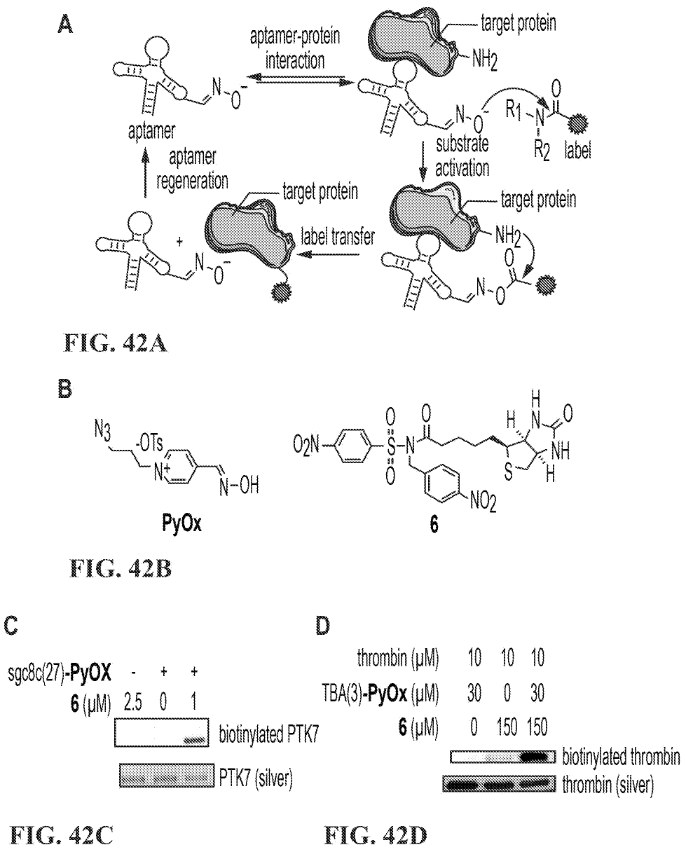

As a complementary approach to aptamer-directed affinity labeling, we have demonstrated aptamer-catalyzed covalent labeling of target proteins. This is based on successes with organocatalyst-modified small molecule ligands that activate substrates within the vicinity of the target protein. In our derived approach (FIG. 42A), the catalyst-functionalized aptamer selectively binds to the target protein and facilitates acyl transfer to a proximal nucleophilic residue only in the presence of an N-acyl sulfonamide (NASA) donor ligand. With this approach, we intend to enhance the degree of selective native protein labeling with covalent aptamers, and we do not need to synthesize different aptamers with different warheads and different labels to be transferred, as one catalyst-bearing aptamer can be combined with differently tuned electrophiles and transfer motifs (e.g., biotin, fluorophores, etc) in a mix-and-match approach based on the desired application. To this end, we have synthesized the pyridinium oxime PyOx (FIG. 42B) organocatalyst as it has demonstrated high efficiency in small molecule-mediated electrophile-activating labeling compared to its DMAP counterpart. Additionally, we synthesized the NASA biotin reagent 6. The catalyst was installed at the previously determined optimal labeling position on sgc8c (SEQ ID NO: 17) and TBA (SEQ ID NO: 1).

We were able to transfer biotin to the PTK7 target in the presence of sgc8c(27)-PyOx and ligand 6 (FIG. 42C). Biotinylation of PTK7 was analyzed by a simplified western blot with SA-HRP and total protein was visualized with silver stain. As a second example, we incubated thrombin with PyOx-functionalized TBA (TBA(3)-PyOx) for one hour at 37° C. The transfer reaction proceeded for five hours following addition of 6 prior to biotinylation analysis by simplified western blot with SA-HRP. Total protein was visualized with silver stain. From this, we observed successful and efficient electrophile-activating transfer of biotin to thrombin (FIG. 42D) and importantly, minimal labeling with just the NASA donor ligand 6 in the absence of aptamer.

### Example 6 Covalent SELEX

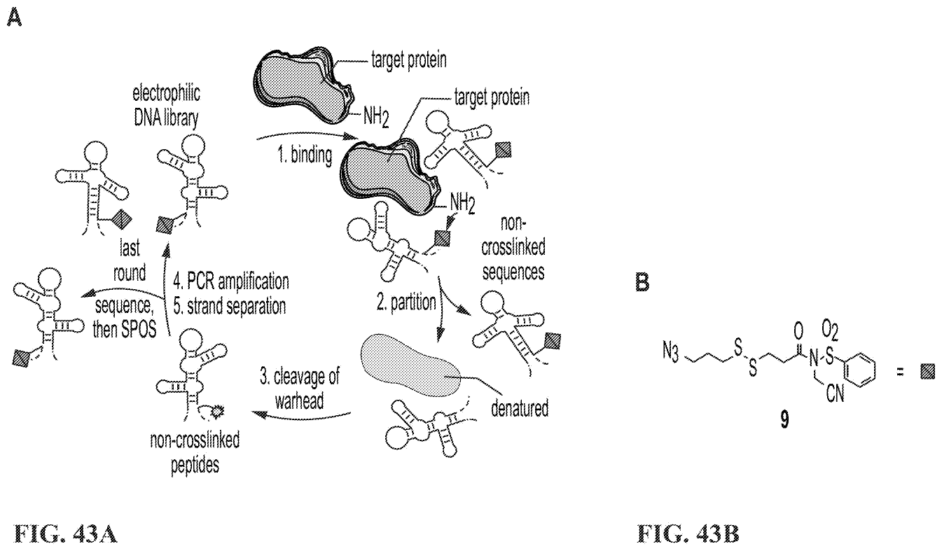

In order to generate covalent aptamers de novo for any protein target, and not having to reprogram existing aptamers, we are developing “covalent systematic evolution of ligands by exponential enrichment” (covalent SELEX or coSELEX)', an in vitro aptamer selection process with distinct advantages to traditional SELEX (FIG. 43A). Two approaches are conceivable when placing the electrophilic warhead onto the oligonucleotide: 1) statistical/random incorporation throughout the sequence, or 2) incorporation at a defined site, such as one of the termini. The covalent selection step allows for the aptamer to selectively bind or label a single nucleophilic residue and allow the creation of user-defined sites and targets, broadening the scope of the new methodology. Our selection strategy reduces the number of selection cycles necessary to generate efficient aptamers due to increased stringency in the wash and partitioning steps, as well as the additional selectivity filter imparted by the covalent bond formation. Since no specialized equipment is needed, covalent SELEX can be performed by any lab and is easily extended to related approaches such as “cell SELEX”. Although we have chosen to utilize DNA aptamers for our selection due to their stability, we expect that a similar approach could also be applied to selections of RNA ligands, as well as non-natural nucleic acids (XNA).

In a first approach, the alkyne was installed on the forward primer at the 5′ end of the randomized region. This approach guarantees that a single warhead can be installed on each oligonucleotide and allows for easy installation of amine-reactive warheads, such as LDNASA. Toward this end, we synthesized a primer-extended TBA(3)-S1, (pr-TBA), allowing for [3+2]cycloaddition of the “inverted”, cleavable disulfide N-acylsulfonamide electrophile 9 (FIG. 43B) to the aptamer. Thrombin was selected as a well-established target for this initial proof-of-concept. The disulfide bond in this electrophile is easily reduced by β-mercaptoethanol (BME) or dithiothreitol (DTT), allowing for quick cleavage of the aptamer-protein interaction. The forward primer (24 nt) was designed with polymerase recognition in mind, can contain a 5-ethynyl dU (S1 or EdU) nucleotide to allow electrophile conjugation. A phosphorylated reverse primer (24 nt) allows for ssDNA generation through lambda exonuclease degradation, as common in traditional SELEX.

Pr-TBA sequence:
(SEQ ID NO: 21)
5′-TAGGGAAGAGAAGGACATATGAT*GGTTGGTGTGGTTGGTTTTTTT
TTTTTTTTTTTTTTTTT TTTGACTAGTACATGACCACTTGA-3′
(S1 indicated as T*)

The aptamer was extended with a poly-T chain to match the length of a library randomized region (40 nt). It was then purified via HPLC, the TMS group was deprotected from the alkyne using TBAF, and the oligo was purified by HPLC once again. The alkyne modified pr-TBA was then conjugated to the crosslinking N-acylsulfonamide warhead 9 via a click reaction. The conjugation and the functionality of the warhead were then confirmed through successful labeling (>90% yield) with a large PEG-amine molecule (Sigma 06679) at 25 mM for 4 h at 37° C. in PBS pH7.4.

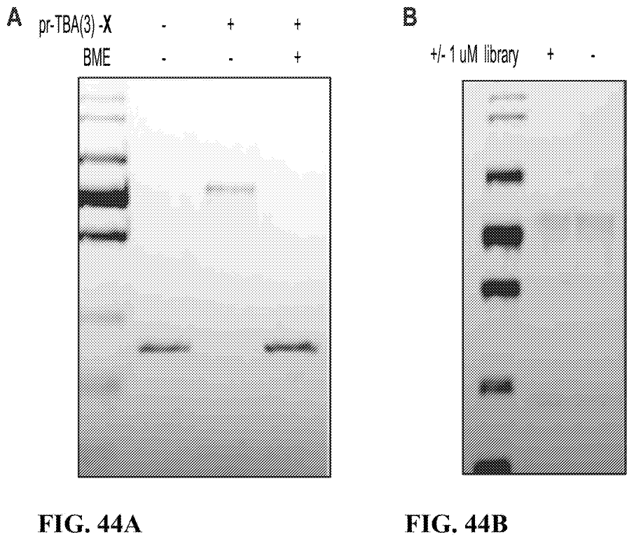

We validated the ability of the cleavable, inverted aptamer pr-TBA(3)-9 to crosslink and subsequently be cleaved from thrombin (FIG. 44A). For this experiment, 300 nM of thrombin was incubated with 1 μM of pr-TBA(3)-9 in PBS pH 7.4 for 1 hour at 37° C. Samples were diluted with Laemlli buffer with or without the reducing agent BME, analyzed by denaturing SDS-PAGE and stained with a silver stain. From this, we observed the expected aptamer-protein crosslinking and subsequent cleavage of the covalent interaction as indicated by the shift in molecular weight. To simulate a SELEX selection, the experiment was repeated using an 10-fold excess of a randomized oligonucleotide library (1 μM), pr-TBA(3)-9 (100 nM), and thrombin (300 nM) in pH 7.4 for 1 hour at 37° C. Samples were diluted with Laemmli buffer without BME and analyzed by denaturing SDS-PAGE and stained with silver stain. We observed the expected aptamer-protein crosslinking even in the presence of the excess random oligonucleotides (FIG. 44B).

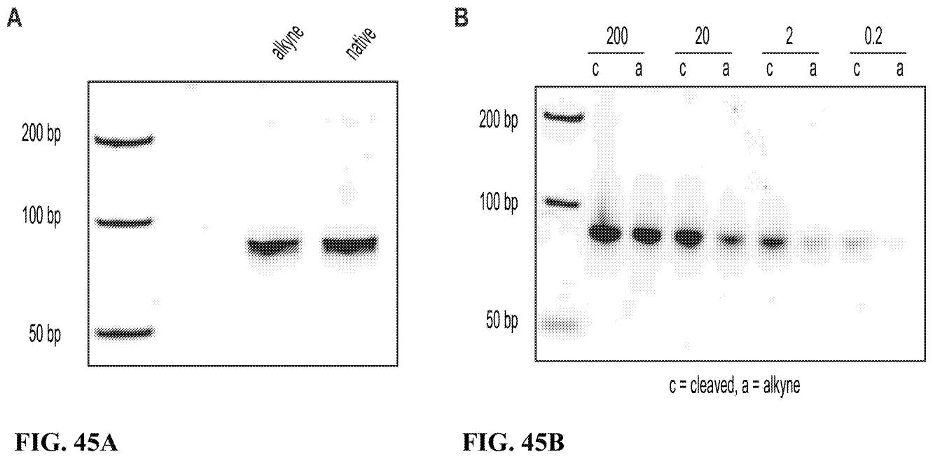

With the successful crosslinking of pr-TBA(3)-9 to thrombin in the presence of excess oligonucleotides, our next step was to ensure that the modified S1-containing library can be accepted by Taq polymerase during PCR amplification. The ds-DNA PCR product of both the native aptamer and the alkyne-modified aptamer were analyzed on a native-PAGE gel and visualized using SYBR gold (FIG. 45A). Our findings indicate no difference in amplification efficiency was observed when the modified primer was compared with an unmodified one. The next step was to ensure the “scarred” aptamer, or pr-TBA(3)-9 cleaved through reduction, could also be accepted by Taq polymerase during PCR amplification (FIG. 45B). A 10 μL, 24 μM solution of pr-TBA(3)-9 was reduced by incubating with 100 mM of dithiothreitol (DTT), another reducing agent capable of cleaving disulfide bonds, for 5 minutes at 95° C. Cleavage was quenched via ethanol precipitation and analyzed via HPLC, showing complete cleavage of warhead. The dsDNA product of the alkyne-modified and scarred aptamers were analyzed on a native-PAGE gel and visualized via SYBR gold. Interestingly, the alkyne-modified aptamer seemed to be more efficiently amplified at lower concentrations.

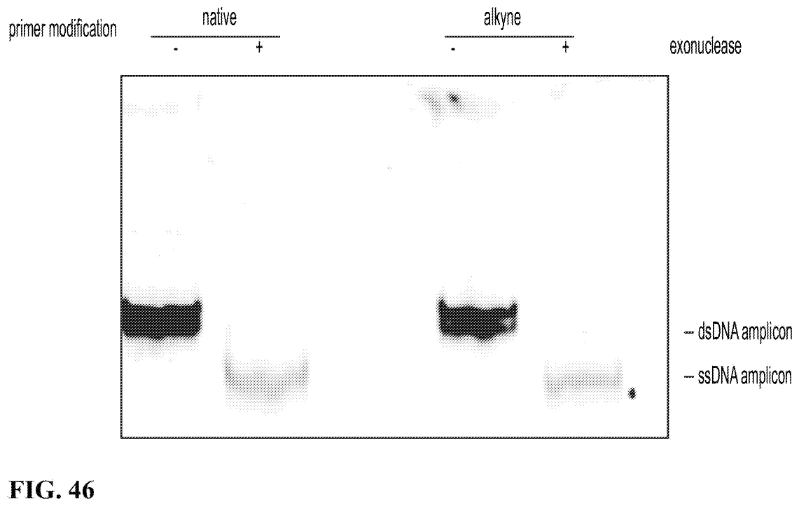

To utilize the amplified oligonucleotides for the subsequent binding step, the strands first had to be separated. To achieve this, a new reverse primer was purchased that contained of 5′ phosphate group. This would allow specific digestion of the antisense strand using lambda exonuclease. Once amplification was repeated using the new primer, the amplicons were incubated at a concentration of 1 μM with 5 U of the exonuclease in 20 μL of NEB buffer (provided with the nuclease) for 2 h at 37° C. The oligonucleotides were then analyzed on a native gel (FIG. 46). It revealed that subjecting the dsDNA amplicon to the nuclease caused a shift in band migration, indicating that the antisense strand was successfully degraded.

Overall, these results lay the foundation for the successful development of a “covalent” SELEX” process where functional aptamers can be eluted after the portioning step through a disulfide reduction.

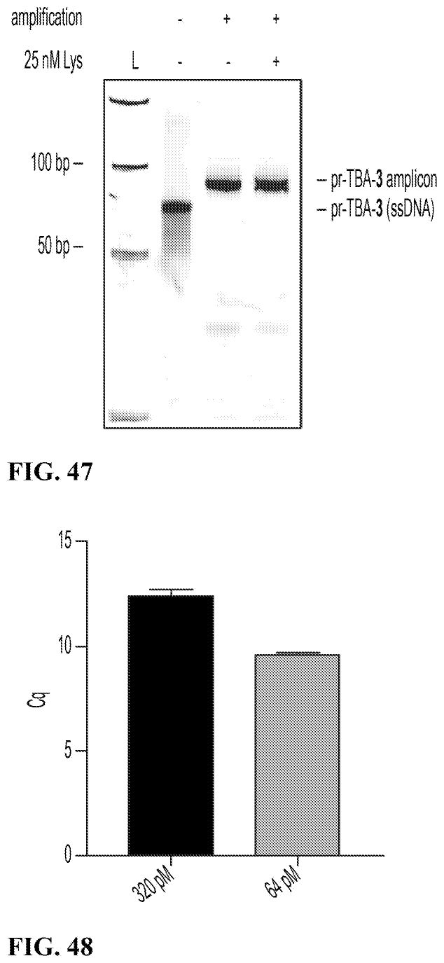

As an alternative release strategy to the aforementioned reductive disulfide cleavage, we also explored post-partitioning aptamer release through target protein degradation by proteinase K. To simulate a partitioning of crosslinking from non-crosslinking sequences, pr-TBA-4 (containing the non-cleavable LDNASA warhead) at 64 pM to 100 nM concentrations was doped into 2 μM of a random oligonucleotide library. The doped library was then incubated with thrombin for 30 min at 37° C. in PBS pH 7.4 to allow TBA-thrombin crosslinking. To separate TBA from remaining non-crosslinking sequences, proteins were immobilized on a nitrocellulose membrane or on an NHS-agarose resin and then washed with a denaturing buffer. Next, to elute crosslinked DNA, proteins were digested using proteinase K. Enriched oligonucleotides were isolated from the eluent using a phenol-chloroform extraction followed by an ethanol precipitation. Since proteinase K leaves a short peptidic scar on the oligonucleotide, amplification of pr-TBA-4 while crosslinked to a Lys residue was tested. Pr-TBA-4 (1 μM) was incubated with lysine (25 mM) for 24 h at 37° C. in PBS pH 7.4. It was then isolated via ethanol precipitation. The crosslinked pr-TBA-4 was then PCR amplified and the amplicons were analyzed by gel and compared to those of non-crosslinked pr-TBA-3 as a control (FIG. 47). No difference in band intensity was detected, indicating that the peptide scar did not interfere with amplification.

Oligonucleotides partitioned through protein reaction with NHS-agarose and proteinase K elution were then quantified via qPCR (FIG. 48). In the absence of pr-TBA-4, no amplification was observed, indicating that non-crosslinking oligonucleotides were all removed in the rigorous wash steps. The same was observed when the library was doped with pr-TBA, which lacks the crosslinking warhead. When the library (2 μM) was doped with as little as 64 pM (6.4 fmol in 100 μL) of pr-TBA-4, amplification was observed, indicating that pr-TBA-4 was enriched with high specificity, and that the mock selection was successful. Overall, this demonstrates successful immobilization, elution, and amplification of crosslinking sequences. Based on protocols for traditional SELEX, the starting library can be comprised of 100 nmol (˜10¹⁵) of random sequences and the first selection round typically retains 0.01-0.1% of the initial library (increases in subsequent rounds). We therefore expect to retain at least 100 pmol of oligonucleotides after the first selection round: several orders of magnitude above our 6.4 fmol amplification/detection limit.

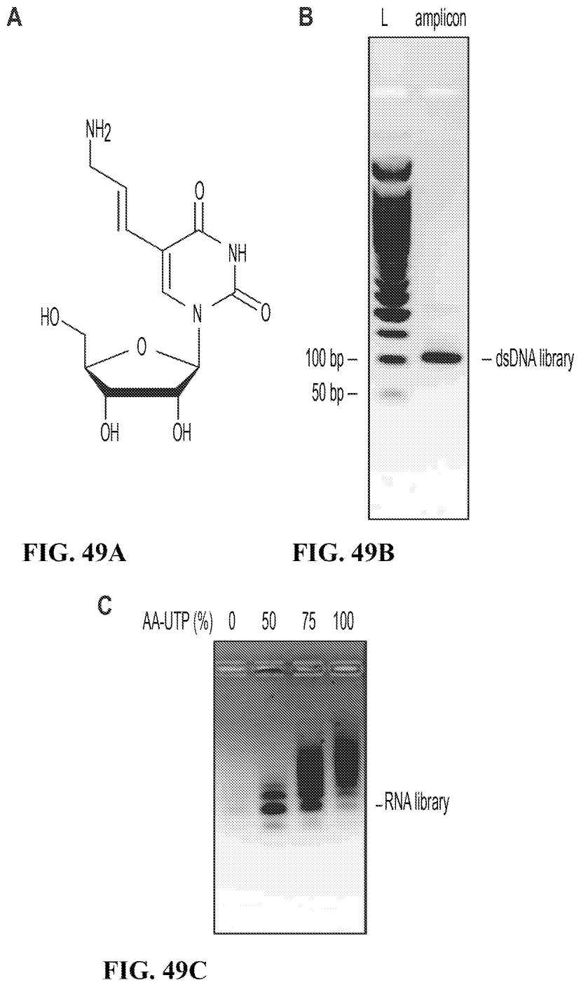

As an alternative covalent SELEX approach, we also laid the foundation for generating RNA-based aptamer libraries with electrophiles statistically dispersed via amine-modified thymidine nucleotides throughout a randomized sequence oligonucleotide library for subsequent warhead installation. This was achieved by enzymatically incorporating amino allyl uridine triphosphate (AA-UTP, ThermoFisher R1091, see FIG. 49A) into a randomized RNA library through transcription. Generation of the library was achieved by using a randomized ssDNA library of 40 nucleotides flanked by forward and reverse primer regions and a 5′ T7 promoter region for transcription (5′ TAG GGA AGA GAA GGA CAT ATG ATN NNN NNN NNN NNN NNN NNN NNN NNN NNN NNN NNN NNN NNN NTT GAC TAG TAC ATG ACC ACT TGA 3′) (SEQ ID NO: 22). A forward primer containing the T7 promoter region (underlined) (5′ TTCAGGTAATACGACTCACTATAGGGAAGAGAAGGACATATGAT 3′) (SEQ ID NO: 23) and a reverse primer (5′ TCAAGTGGTCATGTACTAGTCAA 3′) (SEQ ID NO: 24) were purchased as well.

To prepare the ssDNA library for transcription, it was first amplified via PCR to generate dsDNA, which was analyzed on an agarose gel (FIG. 49B). The gel revealed a single band at the expected molecular weight, indicating successful amplification. The dsDNA library was then transcribed using T7 RNA polymerase in the presence of various UTP to AA-UTP ratios. Transcripts were analyzed on an agarose gel to determine whether AA-UTP interferes with transcription (FIG. 49C). The gel revealed that AA-UTP incorporated efficiently. Interestingly, at high concentrations, AA-UTP caused the oligonucleotide band to smear on the gel. We hypothesized that the positively charged amine interacted with the negatively charged phosphate backbone or partially reduced overall charge, slowing the oligonucleotide migration.

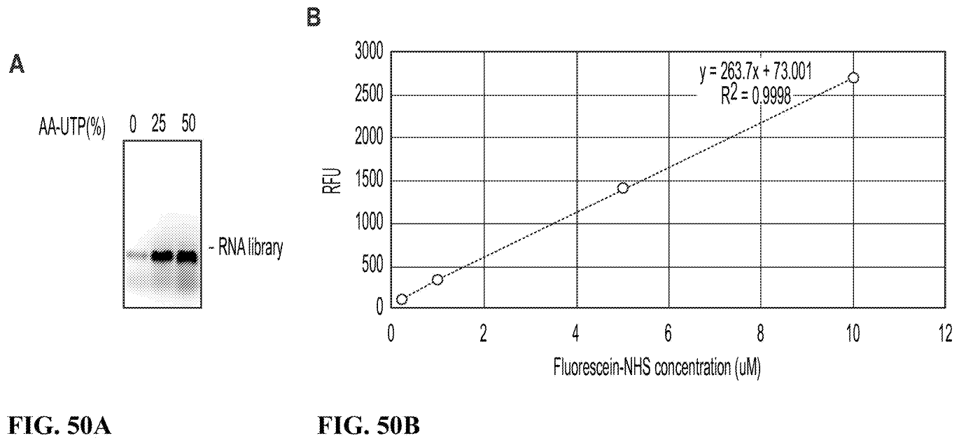

We next aimed to add AA-UTP to the transcription reaction such that, on average, a single AA-UTP would be incorporated into the randomized region of the library. After transcription, the AA-UTP containing RNA was conjugated to fluorescein-NHS (under basic conditions. Oligonucleotides were isolated via ethanol precipitation followed by denaturing PAGE. Bands were visualized via fluorescence imaging (FIG. 50A) and oligonucleotides were extracted and desalted via ethanol precipitation. Gel purification was performed to remove as much fluorescein-NHS as possible. The concentration of oligonucleotide was determined via absorbance at 260 nm while that of fluorescein was determined via absorbance at 494 nm. Based on these concentrations, the number of fluoresceins per randomized region was determined to be 6.6, 12.1, and 24.0 using 0%, 25%, and 50% AA-UTP, respectively. These results demonstrated that the background from this analysis method was too high to obtain accurate results, since it significantly overestimates the number of AA-UTP incorporations. We therefore determined fluorescein concentration using fluorescence instead of absorbance, since fluorescence measurements are more sensitive. A standard curve was generated using fluorescein-NHS (FIG. 50B). The transcription experiment was repeated and fluorescence was measured on a plate reader. Using this approach, the number of fluoresceins per randomized region was 0.26, 0.99, and 1.62 using 0%, 25%, and 50% AA-UTP in the transcription reaction, respectively. The number of fluoresceins should be 0 at 0% AA-UTP. When adjusting for background, the number of fluoresceins at 25% and 50% are 0.73 and 1.36, respectively. These result sets the stage for direct electrophilic warhead installation via amine modification or via an alkyne intermediate, as before, followed by protein target incubation, partitioning via NIH-agarose, and binding aptamer elution via proteinase K treatment.

### 7. Example 7: Protein Detection and Tracking

Aptamer-mediated covalent conjugation provides a powerful approach to monitor proteins and their levels. It is highly versatile, as our covalent aptamer methodology can be tailored to detect proteins at a wide range of concentrations, using different modalities, such as fluorescence, luminescence, or radiography (FIG. 21). In addition to reducing the high costs associated with antibodies and well-documented batch-to-batch reproducibility issues, the workflow of our approach is significantly faster.

By slightly modifying the methodology, we tailored this approach towards the detection of proteins at sub-nanomolar concentrations, by utilizing ³²P-labeled aptamers. We synthesized the “inverted” N-acylsulfonamide electrophilic warhead 4 (see FIG. 16). In contrast to the label-delivering warhead, 12 reacts selectively with proximal lysine residues on the target protein in a manner that covalently crosslinks the protein and DNA aptamer, rather than transferring a label. We demonstrated aptamer-protein covalent crosslinking using the known TBA-thrombin interaction (FIG. 15A). Crosslinking was fast (t_{1/2}=13.6 min using 0.5 μM TBA(3)-12) and protein modification was virtually complete (˜90%) within 1 h (FIG. 15D). Notably, only a single aptamer was conjugated to thrombin, likely due to the crosslinked nucleic acid sterically blocking other aptamers from associating with the protein. Complete selectivity to thrombin was confirmed in human serum using a fluorescein-modified version of TBA(3)-12 (FIG. 15C), matching our results for TBA(3)-1 (FIG. 11C). ³²P-endlabeled TBA(3)-12 allowed for selective thrombin detection in human serum at femtomolar concentration, using SDS-PAGE radiography followed by band integration (FIG. 21A). Lower protein concentrations can most likely be determined since ³²P-oligonucleotides have an estimated detection limit of 1 μM.

Numerous aptamers against cell surface proteins have been generated and have been applied in diagnostics, drug delivery, and therapeutics by dozens of labs. The data provided herein shows that covalent aptamers can also label membrane-bound proteins on live cells, thus enabling their detection as well as other unique applications. We selected protein tyrosine kinase 7 (PTK7), a biomarker of colon, lung, and gastric cancers, as a target. A well-established and highly specific PTK7 binding aptamer (P7BA) bearing a biotin-delivering warhead 1 at position 27 was synthesized and incubated with HEK293T cells (transfected with pcDNA3-PTK7-VSV). Cells were lysed and analyzed via a western blot using a streptavidin-horseradish peroxidase (SA-HRP, ThermoFisher) conjugate (FIG. 27). Cell surface labeling of PTK7 was selective since it was only observed in transfected cells.

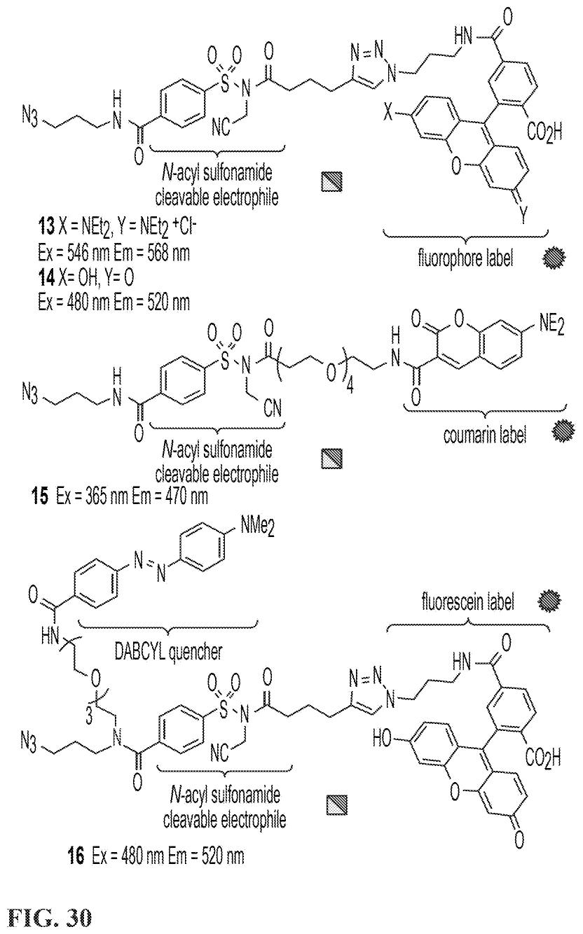

To reduce experimental steps and enable multiplexed, simultaneous detection of different proteins, we can synthesize the photochemically distinct electrophiles 13 and 14 (FIG. 30), we already have the coumarin 15 in hand. These electrophiles can be conjugated to TBA, ABA, and PBA, respectively. Protein labeling can be conducted in serum and HEK293T lysate, and both the target (e.g., thrombin, IgG1 antibody, or PSMA; 0-300 nM) and the aptamer (50-500 nM) can be titrated in order to identify the detection limit and generate standard curves for selective labeling. Labeling can be performed with individual proteins as well as mixtures. SDS-PAGE separation and multiplexed gel imaging (Chemi-Doc), followed by band integration, can demonstrate rapid detection of multiple native proteins at low concentrations in complex mixtures. Additional fluorophores, e.g., cyanine dyes and BODIPY analogs, can also be used to detect proteins in a spectrally resolved fashion.

### 8. Example 8: Targeted Immune Response Elicitation

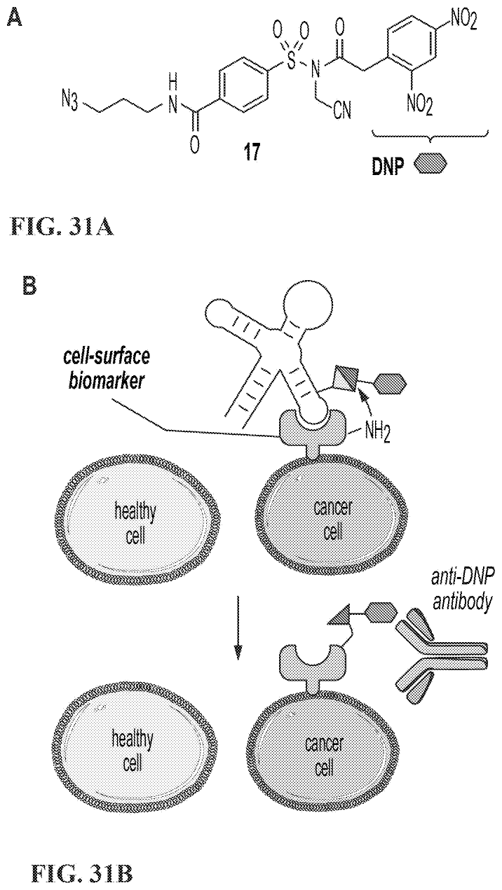

Binding membrane proteins with certain small molecule ligands, called haptens, can redirect an immune response towards normally non-immunogenic cells by recruiting endogenous antibodies capable of binding the hapten. A common hapten is 2,4-dinitrophenyl (DNP), which has been fused to several small molecule ligands and is recognized by approximately 1% of circulating antibodies. Once bound to its target, the antibody-DNP-cell ternary complex elicits opsonization or cytotoxic T cell activation. To expand the target repertoire of this approach and to capitalize on the permanent covalent target engagement, we can synthesize the electrophile 17 and selectively deliver DNP to cell surfaces expressing a cancer biomarker (FIG. 31). The irreversible conjugation can enable target engagement times that match the protein's turnover rate, giving the immune system time to mount an adaptive response.

Specifically, we can target the prominent prostate-specific membrane antigen (PSMA) and modify the PSMA-binding aptamer (PBA, Table 1) with electrophile 17, followed by validation and optimization as described herein. We can incubate PBA with LNCaP cells, as well as PSMA-deficient DUl54 control cells, for 1-3 h to allow for complete handle transfer. Cell-selective DNP delivery can then be confirmed via flow cytometry using an anti-DNP antibody (Sigma). Immune response elicitation can be assessed in a dose-dependent manner by measuring cellular cytotoxicity using a commercially available assay (Promega, ADCC Reporter Bioassay) in response to the presence of the antibody and the aptamer. This assay utilizes natural killer (NK) cells expressing the Fc receptor. Once the receptor binds the anti-DNP antibody decorating the LNCaP cells, it triggers the NFAT pathway, which in turn drives expression of a luciferase reporter. We expect that multiple DNP transfers to the biomarker protein can aid in antibody recruitment and that immune cell activation can occur exclusively in response to PSMA-positive LNCaP cells, demonstrating the ability to elicit a cell-specific immune response. Controls can include a scrambled sequence aptamer bearing electrophile 17 and a PSMA aptamer that is incapable of transferring its conjugated DNP. Based on our successful labeling of PTK7, we can also test the utility of its aptamer, P7BA-17, for DNP transfer and an immune response to HeLa and HEK293T cells as positive and negative controls, respectively.

A crystal structure of DNP bound to an anti-DNP antibody (PDB 1BAF) reveals that the only lysine proximal (19 Å) to DNP is inaccessible to the electrophile due to other amino acids blocking the needed reaction trajectory. Consequently, we expect that covalent labeling exclusively occurs at PSMA rather than the antibody.

### Example 9: Targeted Protein Degradation

While selection (SELEX)-based approaches to aptamer discovery open up the entire proteome for targeting, aptamers are purely selected based on binding and not based on inhibition of their protein targets. Thus, they may not block enzymatic function due to their binding site or because they get outcompeted by endogenous protein binding partners. PROTACs and LYTACs have emerged as promising technologies that quickly and effectively degrade target proteins, thereby offering a unique solution. PROTACs are heterobifunctional molecules that recruit an E3 ligase to a protein of interest. Ternary complex formation initiates degradation through ubiquitin transfer to the target protein, effectively hijacking the cellular ubiquitin-proteasome degradation pathway. While PROTACs capitalize on intracellular protein degradation pathways, LYTACs offer a complementary approach to target the remaining ˜40% of the proteome that is either membrane-bound or extracellular. LYTACs are antibody-glycopeptide conjugates that function by recruiting the protein of interest to the cation-independent mannose-6-phosphate receptor (CI-M6PR), which shuttles it to the lysosome for degradation.

We describe developing a novel nucleic acid-based protein degradation platform that expands the targeting repertoire currently offered. Our covalent labeling methodology can serve to decouple the aptamer-protein binding kinetics from the protein degradation rate, which are inversely correlated, enabling the utilization of aptamers with otherwise too brief target engagement times.

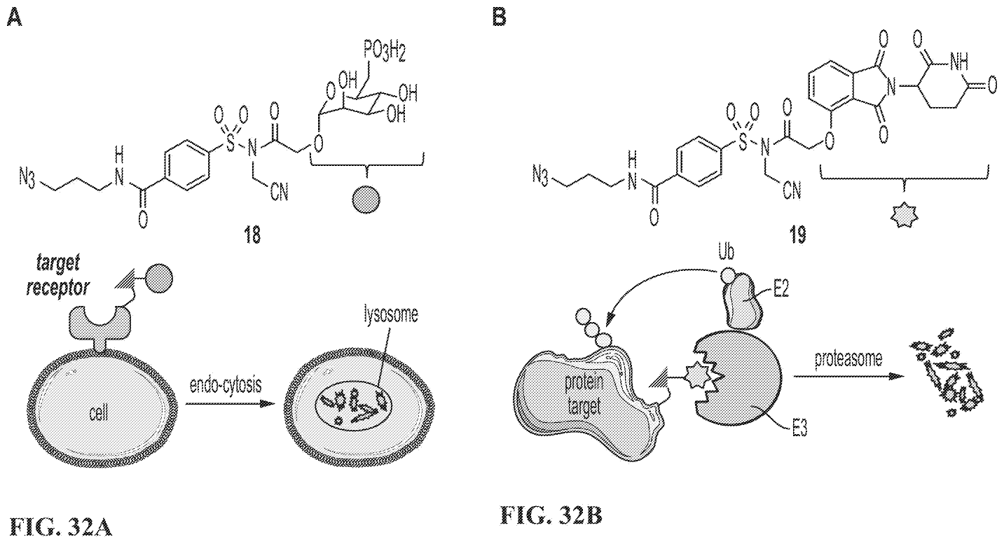

In order to develop nucleic acid-based LYTACs, we can target PTK7 using P7BA. We can synthesize the electrophile 18, which can transfer a mannose-6-phosphate onto PTK7, thereby tagging it for lysosomal degradation (FIG. 32A). The aptamer sgc8c-27 can be incubated with Jurkat or Nalm-6 cells and protein depletion can be monitored over 4 h by western blot (anti-PTK7 antibody, Abcam). To demonstrate that covalent labeling is essential, a sgc8c-27 analogue lacking the CH₂CN group that cannot transfer its label can be generated and used as a control. Based on previous antibody-based LYTAC experiments, we expect that target protein degradation can be achieved within just 1 h.

To establish intracellular applicability of our approach, the FOXM1 binding aptamer (FBA, Table 1), a 42-nt phosphorothioate DNA aptamer with 65 nM affinity, can be our first nucleic acid-based PROTAC proof-of-concept. A total of 15 aptamers can be synthesized. The FOXM1 transcription factor activates a network of proliferation-associated genes and is overexpressed in cancers of the prostate, breast, lung, ovary, colon, and pancreas. Importantly, FOXM1 depletion induced cell cycle arrest and inhibited cell invasion in hepatocellular carcinoma. FOXM1 contains 8 lysine residues, which makes it an attractive target for both covalent labeling and ubiquitination. The FBAs can be conjugated to the biotinylating electrophile 1, transfected (Lipofectamin™ RNAiMAX, ThermoFisher) into MDA-MB-436 breast cancer cells expressing FOXM1, and incubated for 0-16 h. Cell lysate analysis by western blot using both streptavidin-HRP and an anti-FOXM1 antibody (CST) can confirm aptamer specificity. To build upon this system, we can synthesize the electrophile 19, capable of delivering an established CRBN E3 ligase ligand, and conjugate it to FBA (FIG. 32B). Covalent conjugation of the proteasomal/lysosomal machinery recruiting ligand to the target can abrogate catalytic activity typically associated with PROTACs; however, since turnover numbers are low (2.0-3.4 in vitro), we expect that the loss of catalysis can only have a minimal effect on degradation. FOXM1 degradation by the E3 ligand delivering aptamer FBA-7 can be tested in MDA-MB-436 cells by monitoring FOXM1 levels via western blot over 24 h. Based on PROTAC kinetic analyses we expect to achieve maximal degradation within 4 h, with the proteasomal machinery being rate-limiting. We can utilize epoxomicin (1 μM; Sigma) and a non-electrophilic FBA aptamer lacking the CH₂CN moiety as controls to confirm FOXM1 ubiquitination by western blot and to validate that degradation occurs via a proteasomal pathway.

### 10. Example 10: Virus Labeling and Cell Entry Neutralization

The current SARS-CoV-2 outbreak and COVID-19 pandemic has posed a serious threat to public health, making the development of reagents capable of detecting and neutralizing the virus highly desirable. S protein plays a critical role in viral attachment and entry by targeting angiotensin-converting enzyme 2 (ACE2) on host cell surfaces through its receptor-binding domain (RBD). S protein-specific as well as RBD-specific DNA aptamers (RBAs) have been recently identified. The RBAs display KDS of 6 nM and 20 nM (Table 1). Using RBA-1, we already performed a position-dependent RBD biotinylation. We found that, like TBA-1, biotinylation was highly dependent on warhead placement, with position 19 being the most efficient, followed by positions 15 and 43. Crosslinking RBA-12 follows these trends as well. We can modify these aptamers with our “inverted” electrophilic warhead 12 at several positions. Consequently, the aptamer can covalently bind to one of the eleven lysine residues on RBD and sterically block binding to ACE2 through permanent target occupation. The electrophilic aptamers (200 nM) can be individually incubated with RBD (1 μM) for 1 h at 37° C. in PBS pH 7.4. Covalent conjugation can be analyzed by SDS-PAGE, as previously demonstrated for TBA (FIG. 15). When aptamer crosslinking does not induce an electrophoretic shift, the oligo can be ³²P-radiolabeled for detection. Efficient aptamers RBA-15, -19 and -43 can be incubated with RBD in human serum at 37° C. for 0.25-2 h. Conjugation can be analyzed via western blot using an RBD antibody (Mybiosource), and based on our thrombin results, we expect >90% conjugation within 1 h.

To test whether aptamer covalent conjugation to RBD neutralizes SARS-CoV 2, viral particles pseuodotyped with S protein can be generated using a HIV lentiviral system split across three plasmids according to a published protocol. Plasmids expressing a luciferase reporter, S protein, and other HIV machinery required for virion formation (BEI Resources) can be co-transfected into HEK293 cells. The widely infectious VSV-G envelope plasmid (Addgene) can be used as a positive control. Virions can be harvested 48 h post transfection and S protein expression can be confirmed using a luciferase reporter assay (Promega). Virion containing supernatants can be treated with RBA-12, RBA, or a scramble control at 10 nM to 1 μM concentrations for 0.25-2 h. They can then be used for single cycle transfection of HEK293 cells constitutively expressing ACE2 (BEI Resources) and wild type as a negative control. After an additional 60 h, cell infection can be analyzed via luciferase activity.

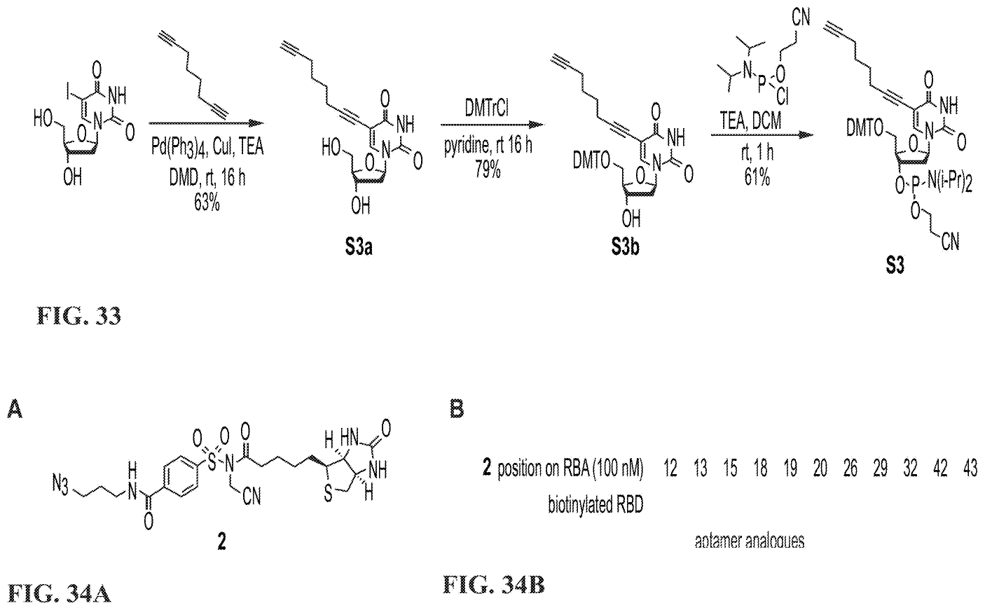

We have modified this approach by converting a reported RBD binding aptamer (RBA) into a covalent aptamer that transfers a functional motif to RBD. To accomplish this, we have synthesized modified RBA derivatives with 11 thymidines individually replaced with the alkyne analogue S3 that we synthesized (FIG. 33). The incorporated thymidines are bolded in the RBA sequence:

(SEQ ID No: 9):
5′-CAGCACCGACCTTGTGCTTTGGGAGTGCTGGTCCAAGGGCGTTAAT
GGACA-3′

We performed a structure-activity relationship (SAR) study through the incubation of RBD (250 nM) with biotinylating aptamers RBA(n)-2 (500 nm) for four hours at 37° C. (FIG. 34). RBD biotinylation was analyzed with a simplified western blot using a streptavidin-horseradish peroxidase fusion (SA-HRP, ThermoFisher). From this, a distinct SAR was observed—aptamers with 2 incorporated at positions 29 and 42 demonstrated the most efficient proximity-induced RBD biotinylaton. Warhead installation at other positions only showed diminished or no target labeling activity.

### 11. Example 11: Detecting and Treating Cancer

a) Covalent Aptamers Targeting c-MET

Signaling pathways involved in cell growth and migration, inhibition of apoptosis, and angiogenesis, are induced by the hepatocyte growth factor (HGF) and c-MET receptor interaction. Both HGF and c-MET are clinical targets for cancer, as the HGF-MET pathway has been correlated to poor prognosis as well as an increased risk for metastases. Due to its clinical relevance, small molecule inhibitors and antibody-based therapeutics targeting c-MET have been developed. A DNA aptamer, CLN0003, has been shown to bind to c-MET, and a truncated derivative, SL1, has demonstrated inhibition of c-MET signaling and as a result, suppression of metastatic behavior of cancer cells. Capitalizing on this, we have incorporated the handle-transferring electrophile 2 into SL1 as a novel approach to hinder the interaction between HGF and c-MET. We site-specifically replaced thymidines at positions 23 and 29 (bolded in sequence below) of the SL1 sequence with the alkyne phosphoramidite S3 prior to their conjugation to the NASA warhead 2:

(SEQ ID NO: 25)
5′-ATCAGGCTGGATGGTAGCTCGGTCGGGGTGGGTGGGTTGGCAAGTC
TGAT-3′

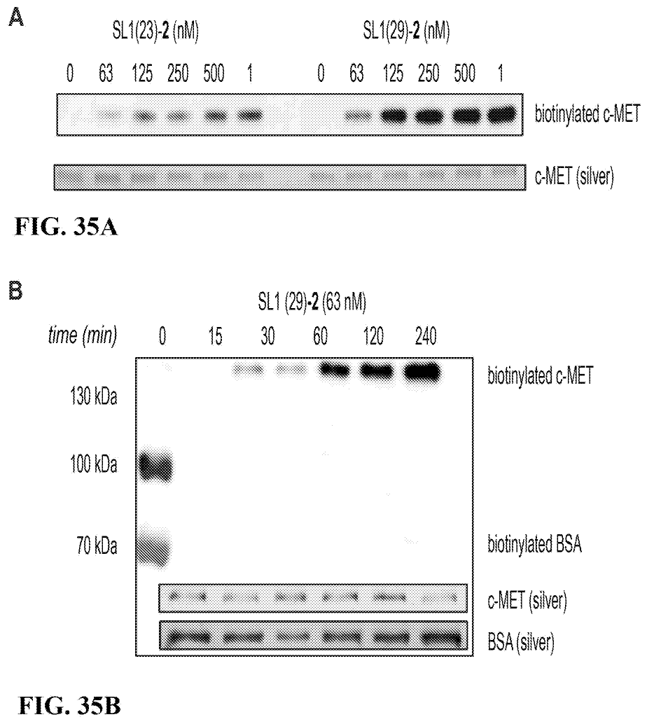

These positions were strategically chosen because they are embedded within the expected G-quadruplex structure of SL1 and thus are likely to interact with c-MET, as G-quadruplex motifs found in nucleic acid structures are frequent interaction points with target proteins. The chemically modified aptamers were incubated at various concentrations (63 nM to 1 μM) with recombinant c-MET (100 nM) in the presence of BSA for one hour at 37° C. (FIG. 35A). c-MET biotinylation was analyzed with a simplified western blot using SA-HRP and total protein was visualized with silver stain. From this, SL1 (29)-2 displayed greater labeling efficiency than SL1(23)-2, with c-MET labeling observed as low as 63 nM concentration of aptamer.

A time-course experiment was performed with SL1 (29)-2 (63 nM) in the presence of c-MET (100 nM) and excess BSA to evaluate the extent and selectivity of the labeling reaction (FIG. 35B). c-MET biotinylation at this low concentration of aptamer was already detected at fifteen minutes, and BSA was not labeled at all. This demonstrated fast and highly selective c-MET biotinylation with our covalent aptamer SL1 (29)-2.

### b) Covalent Aptamers Targeting PTK7

Numerous aptamers against cell surface proteins have been generated and have been applied in diagnostics, drug delivery, and therapeutics by dozens of labs. The data shows that covalent aptamers can label membrane-bound proteins on live cells, thus enabling detection and other applications. We selected protein tyrosine kinase 7 (PTK7), a biomarker of colon, lung, and gastric cancers, as a target. A well-established and highly specific PTK7 binding aptamer (sgc8c) bearing a biotin-delivering warhead 2 at position 27 was synthesized and incubated with HEK293T cells (transfected with pcDNA3-PTK7-VSV).

Protein tyrosine kinase 7 (PTK7) is a catalytically active, transmembrane receptor pseudokinase that plays a role in the Wnt/planar cell polarity pathways and affects numerous essential cellular functions including cell growth, movement, and survival. High expression levels of PTK7 are a biomarker in several cancer types, including leukemia, colon cancer, non-small-cell lung cancer, gastric cancer, and cervical cancer. Thus, PTK7 is an attractive target for anticancer therapeutic development, which is emphasized by the development of PTK7 CAR-T cells as well as the completion of Pfizer's first in-human study of a PTK7-targeting antibody-drug conjugate for the treatment of solid tumors.

As a co-receptor of the Wnt pathway, PTK7 was recently shown to translocate from the membrane to the cytoplasm in response to Wnt3a. Researchers conducting localization studies typically express the proteins of interest fused to reporters (e.g., GFP). This requires genetic manipulation and aberrant protein expression can lead to unwanted artifacts, potentially masking the native role and localization of the protein of interest. To complement genetic engineering methods for studying protein localization, we can utilize P7BA to deliver 16, a turn-on fluorescent probe based on quencher release that should obviate the need for wash steps, to HeLa cells, which endogenously express PTK7. Handle transfer can be quantified via fluorescent imaging and flow cytometry. PTK7 internalization in response to 0-10 μg/mL of Wnt3a (R&D Systems) can be monitored via microscopy, following previous studies, using fluorescent imaging. Controls can include P7BA incapable of transferring 16 and Wnt5a (R&D Systems), which should not induce receptor internalization.

The known PTK7-targeting DNA aptamer sgc8c, has been utilized in various applications including as an imaging probe for the detection of melanoma and as an anti-cancer aptamer-drug conjugate. We have transformed sgc8c into a unqiue covalent aptamer, replacing nine thymidine residues (bolded in sequence below) with the alkyne phosphoramidite S3 and subsequent conjugation to the NASA electrophile 2:

(SEQ ID NO: 16)
5′-ATCTAACTGCTGCGCCGCCGGGAAAATACTGTACGGTTAGA-3′

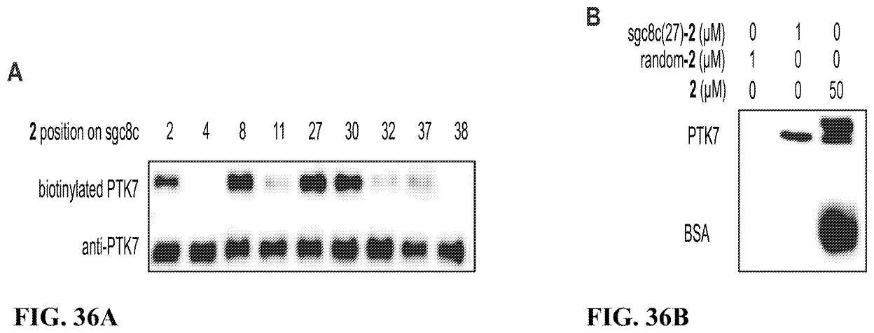

Modified aptamers (500 nM) were individually incubated with recombinant PTK7 (100 nM) for one hour at 37° C. (FIG. 36A). From this, a distinct SAR was observed, with electrophiles installed at positions 8, 27, and 30 showing high labeling efficiency, while warheads at positions 4, 11, 32, 37, and 38 did not perform PTK7 labeling. We further demonstrated the excellent selectivity of sgc8c(27)-2 by incubating it in the presence of PTK7 (100 nM) as well as an excess of BSA (1.5 μM, FIG. 36B). We chose to move forward the position 27 aptamer due to its favorable labeling efficiency, though either sgc8c(8)-2 or sgc8c(30)-2 could have also been used in subsequent experiments.

The aptamer sgc8c was discovered as an oligonucleotide ligand for PTK7 through cell-based SELEX, which is a selection process that enables the assembly of an aptamer library for diseased cell recognition. Because of this, we expected that covalent sgc8c(27)-2 would exclusively biotinylate lysines on the extracellular domain of PTK7 (amino acids 31-703). To evaluate where biotinylation occurs, PTK7 (500 nM) was incubated with and without sgc8c(27)-2 for one hour at 37° C., separated on a denaturing gel, and submitted for mass spec sequencing. Results indicated three unique biotinylated peptides that contained modified lysines K132, K501, and K636, which are in fact located on the extracellular domain of PTK7.

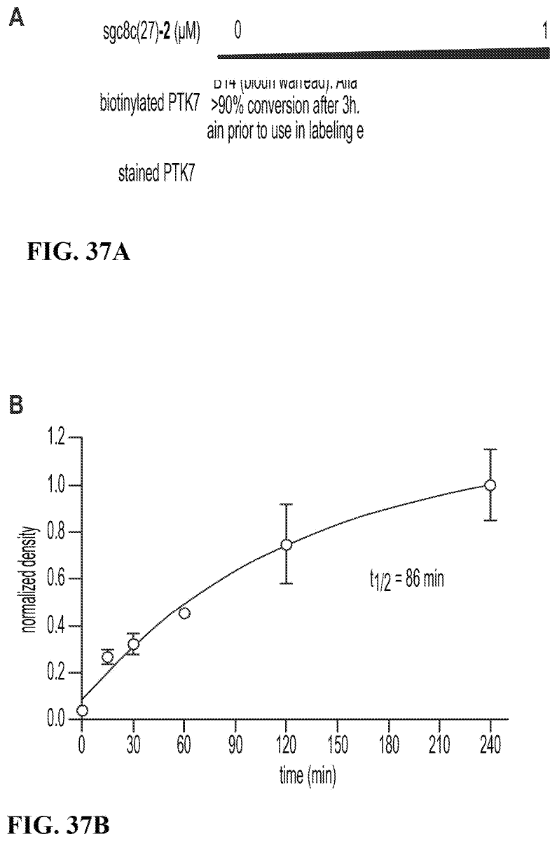

The optimal labeling that could be achieved with sgc8c(27)-2 was determined by incubating various concentrations with PTK7 (100 nM) for one hour at 37° C. (FIG. 37A). Aptamer-mediated biotinylation of PTK7 is observed at concentrations as low as 63 nM, with a plateau being reached at 500 nM aptamer. For the examination of labeling kinetics, a time-course was performed by incubating sgc8c(27)-2 and PTK7 (1:1, 100 nM). This experiment showed that PTK7 biotinylation occurred within fifteen minutes and continued over four hours with a t_{1/2} of 1.5 hours (FIG. 37B).

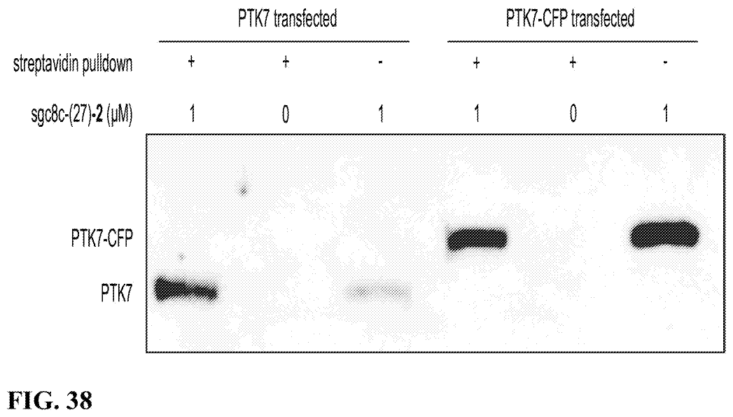

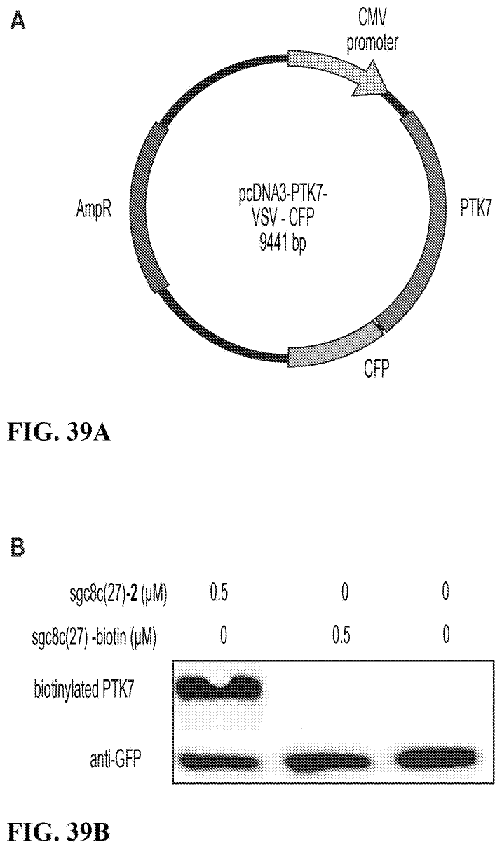

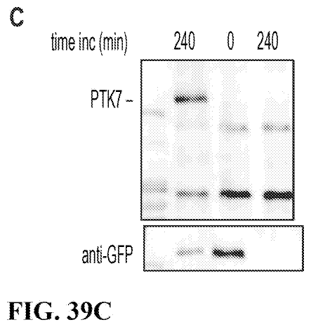

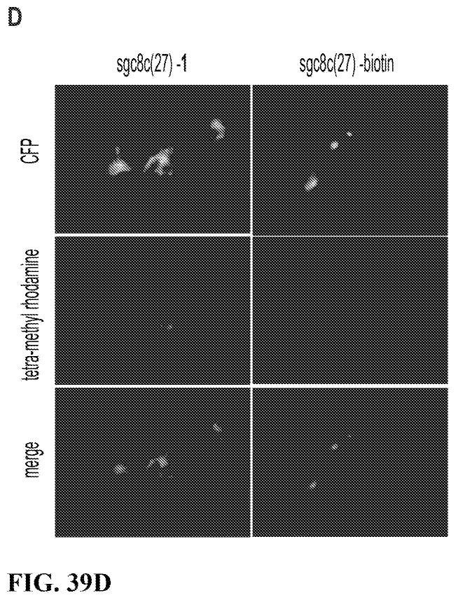

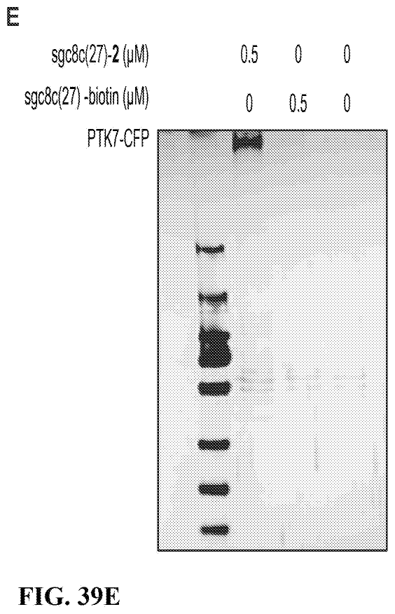

Next, we demonstrated the first use of covalent aptamers to modify protein on cell surfaces using our modified sgc8c(27)-2 and mammalian cells transiently expressing a PTK7-cyan fluorescent protein (CFP) fusion construct (FIG. 39A). The CFP tag was added to the C-terminus of PTK7 as it is cytosolic and therefore should not interfere with aptamer binding as we have determined that biotinylation occurs on the extracellular domain. A labeling experiment was performed in cells expressing both constructs and subsequent pulldown was performed in parallel with PTK7 and PTK7-CFP, which showed comparable labeling of both wild-type and fluorescently labeled protein, indicating that the CFP tag does not interrupt the aptamer-protein interaction (FIG. 38).

Once the expression construct was confirmed to express in mammalian cells, cell surface modification was tested with covalent aptamers by incubating HEK293T cells expressing PTK7-CFP with 0.5 μM of sgc8c(27)-2 or sgc8c(27)-biotin—the same aptamer with a non-transferable biotin serving as a negative control—for one hour. Cells were lysed, biotinylated proteins were pulled down with streptavidin resin, and subsequently analyzed via a simplified western blot, with an additional anti-GFP blot to confirm PTK7-CFP expression (FIG. 39B). In contrast, no labeling is observed when the HEK293T cells are not transiently expressing the PTK7-CFP fusion construct, again indicating the high selectivity of the covalent aptamers (FIG. 39C). Labeling of PTK7 was only observed when incubated with sgc8c(27)-2 and not with the non-labeling sgc8c(27)-biotin aptamer, validating that label transfer is in fact covalent. Additionally, label transfer was found to be specific to PTK7 at 0.5 μM of aptamer when total protein in pulled-down lysates was visualized with a highly sensitive silver stain (FIG. 39E).

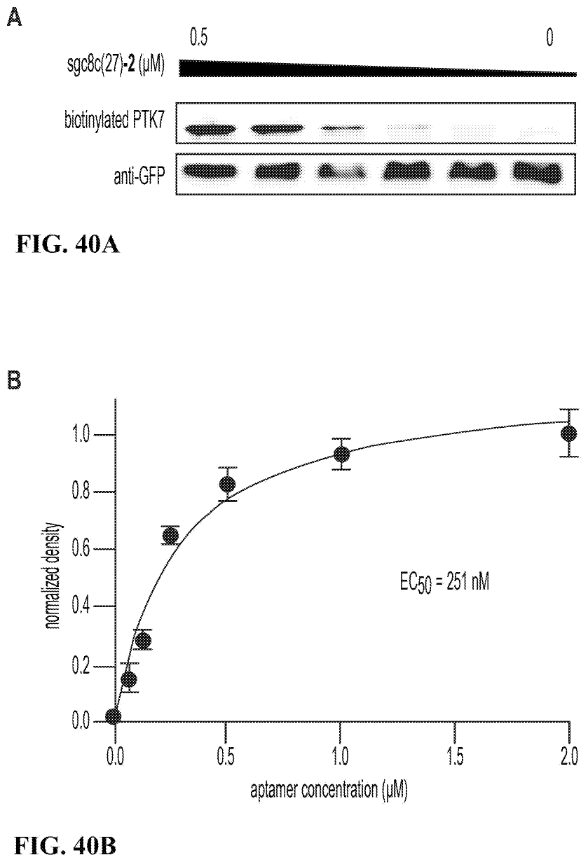

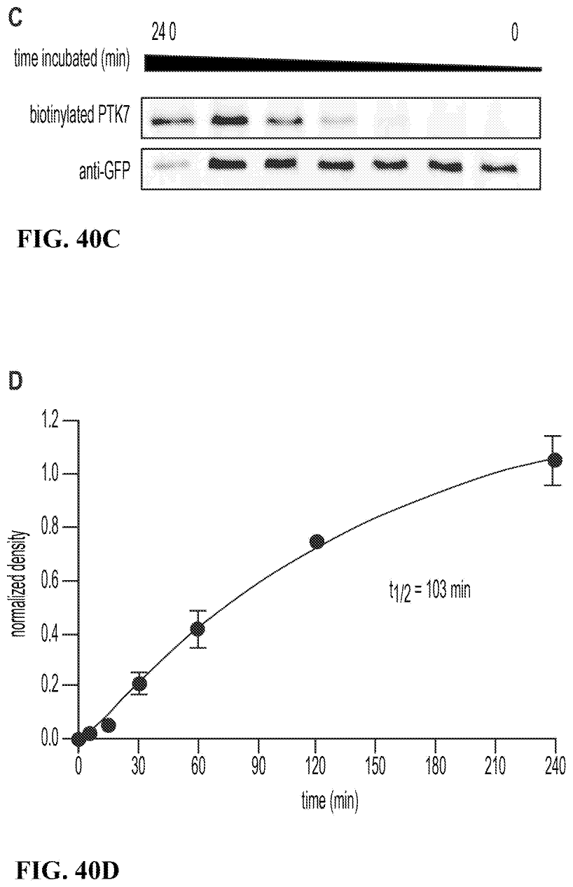

After demonstrating highly specific, aptamer-mediated covalent cell surface labeling, we determined that off-target labeling was not observed at aptamer concentrations as high as 500 nM in HEK293T cells and confirmed that labeling can be achieved at concentrations as low as 31 nM (FIG. 40A). Bands in the western blot were quantified to determine the labeling yield and were normalized to total PTK7 expression in cells, with biotin transfer plateauing out above 500 nM (FIG. 40B). A time-course experiment of covalent labeling of HEK293T cell surfaces using 0.25 μM of sgc8c(27)-2 showed a t_{1/2} of 103 min, matching the test tube experiments (FIG. 40C,D). Overall, we concluded that cell surface labeling proceeded in a very similar manner to the labeling of purified protein.

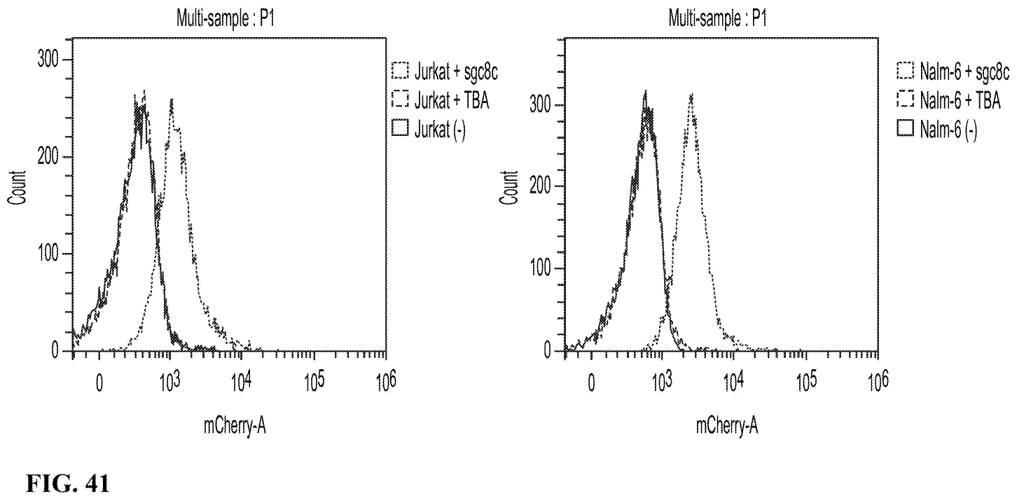

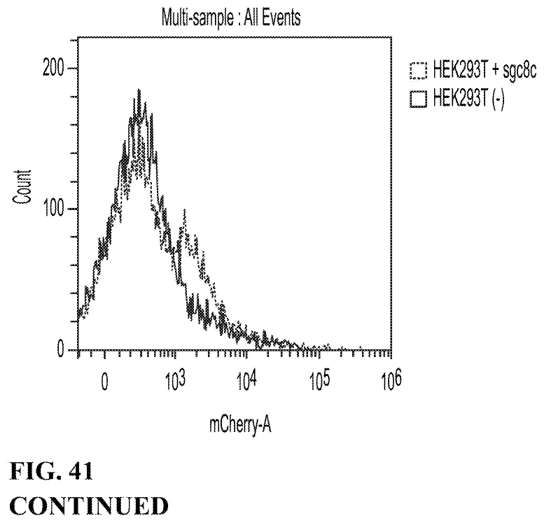

We also validated the ability of our covalent aptamers to selectively label cells that express endogenous levels of PTK7, specifically Jurkat and Nalm-6 leukemia cells (FIG. 41). HEK293T cells and the thrombin aptamer TBA(3)-2 were used as negative controls to demonstrate specificity for both the cell line and the aptamer. The results showed successful labeling of endogenous PTK7, with minimal labeling of non-PTK7 expressing cells.

### Chemical Protocols

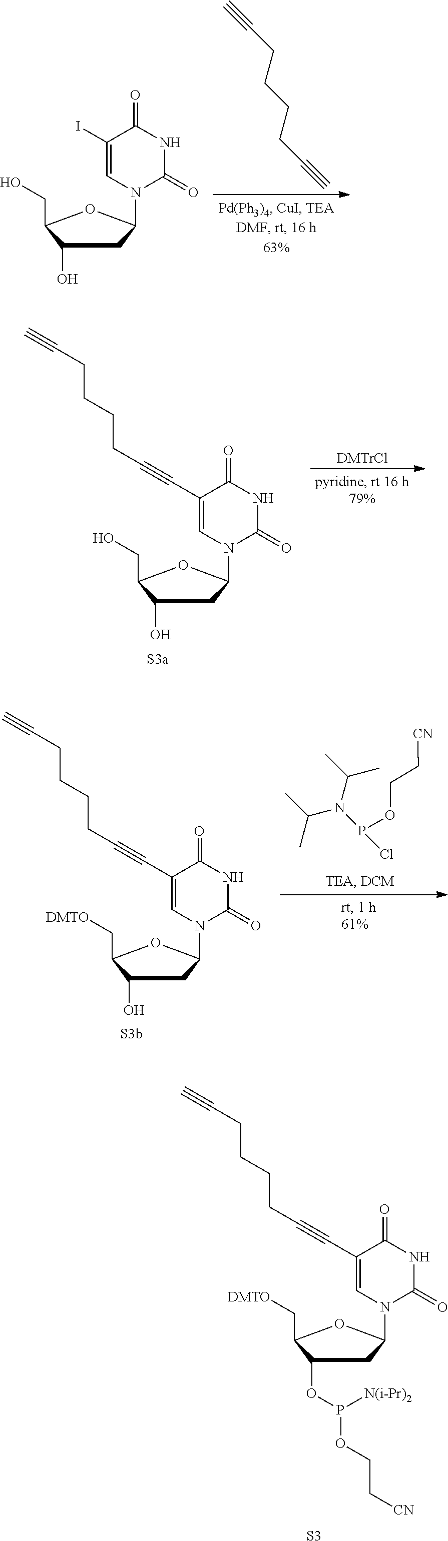

5-(1,7)-Octadiynyl-2′-deoxyuridine (S3a). 5-Iodo-2′-deoxyuridine (33 mg, 0.85 mmol) was dissolved inn DMF (9 mL). CuI (32 mg, 0.17 mmol) was added, followed by Pd(PPh₃)₄ (104 mg, 0.09 mmol), TEA (237 μL, 1.7 mmol), and 1,7-octadiyne (1.13 mL, 8.50 mmol). The reaction mixture was stirred for 16 h at room temperature and concentrated under reduced pressure onto silica gel (1 g). The product was then purified by column chromatography on silica gel using a gradient of 0-5% MeOH in DCM to give compound S3a in 72% yield (203 mg) as a yellow solid. Analytical data matched those previously reported (see Brunner, K.; Harder, J.; Halbach, T.; Willibald, J.; Spada, F.; Gnerlich, F.; Sparrer, K.; Beil, A.; Mockl, L.; Brauchle, C.; Conzelmann, K. K.; Carell, T., Cell-penetrating and neurotargeting dendritic siRNA nanostructures. Angew Chem Int Ed Engl 2015, 54 (6), 1946-9).

5-(1,7)-Octadiynyl-5′-O-DMTr-2′-deoxyuridine (S3b). The nucleoside S3a (123 mg, 0.37 mmol) was dissolved in pyridine (3 mL). DMTrCl (138 mg, 0.41 mmol) was added and the reaction mixture was stirred for 16 h at room temperature. It was then quenched with 1 mL of methanol and stirred an additional 15 min. The reaction mixture was concentrated under reduced pressure onto silical gel (300 mg) and the product was purified by flash chromatography on silica gel using 65% ethyl acetate in hexanes as the eluent. Compound S3b was obtained in 79% yield (186 mg) as a white solid. Analytical data matched those previously reported.¹

5-(1,7)-Octadiynyl-5′-O-DMTr-2′-deoxyuridine phosphoramidite (S3). The DMT ether S3b (135 mg) was dissolved in acetonitrile (6 mL) followed by 2-cyanoethyl N,N,N′,N′-tetraisopropylphosphorodiamidite (133 μL, 0.42 mmol) and 5-(ethylthio)-1H-tetrazole (55 mg, 0.42 mmol). The reaction mixture was stirred for 4 h at room temperature. It was then diluted with DCM (50 mL) and washed with saturated sodium bicarbonate (10 mL). The aqueous layer was then extracted with DCM three times (40 mL). The combined organic layer was dried over anhydrous sodium sulfate (1 g), filtered over cotton, and concentrated in vacuo onto silica gel (500 mg). The product was purified by flash chromatography on silica gel using 35% ethyl acetate in hexanes as the eluent. Compound S3a was obtained in 61% yield as a white solid. NMR and HRMS data match those previously reported.¹

Oligonucleotide Synthesis. Oligonucleotide syntheses were performed using standard β-cyanoethyl phosphoramidite chemistry on an Expedite 8909 DNA/RNA Synthesizer (Distribio, NY, USA). Aptamers were synthesized on a 200 nanomole scale using 500 Å derivatized CPG solid phase supports obtained from Glen Research (VA, USA) using a 5′-dimethoxytrityl

(DMTr)-OFF approach. Synthesis cycles with coupling times of 2 and 10 minutes were used for both unmodified and modified phosphoramidites (OdU respectively, at 0.07 M concentration. Coupling efficiency was monitored by DMTr cation release after each deprotection step—no significant loss was observed following modified phosphoramidite addition to the oligonucleotide. Cleavage of the oligonucleotide from the solid support and subsequent deprotection were carried out with 1 mL of 2 N ammonia in methanol at room temperature for 1 h. Following, concentration in vacuo, the clear oil was resuspended in 1:1 methylamine in ammonium hydroxide (1 mL) and heated at 65° C. for 15 min. The solution was then concentrated in vacuo prior to isopropanol precipitation.

### Biological Protocols

### Recombinant PTK7 Biotinylation

PTK7 (R&D Biosystems) was diluted to 100 nM in DPBS, pH 7.4. The PTK7 solution (2 μL) was further diluted with DPBS (18 μL). For dose-response experiments, serial dilutions of a 10 μM stock of the electrophilic aptamer (sgc8c(27)-2) were generated such that desired concentrations could be achieved when added (2 μL) to a 20 μL total reaction volume. Following aptamer addition, the reaction incubated at 37° C. for one hour (or indicated amount of time). If performing a selectivity experiment, PTK7 was diluted with DPBS (14 μL) and a solution of BSA (Thermo Fisher, 2 μL) was added. To this, the electrophilic aptamer was diluted to 10 μM in milliQ water and then added (2 μL) to the PTK7 solution to achieve a total reaction volume of 20 μL. The reaction incubated at 37° C. for the indicated amount of time.

### Analysis of PTK7 Biotinylation

Following aptamer-mediated biotin transfer, reaction samples were boiled in 6×Laemmli sample buffer (4 μL) for five minutes at 95° C. on a heat block. Recombinant protein samples were separated on a 6% (v/v) SDS-PAGE gel electrophoresis (60 V 20 min, 140 V, 1.25 h). Cellular protein samples were separated on a 10%/6% (v/v) 2-step SDS-PAGE gel electrophoresis (60 V 20 min, 140 V, 1.25 h). Following, proteins were transferred (80 V, 1.75 h) to a PVDF membrane (GE Healthcare) and the membrane was blocked in blocking buffer (5% BSA in 1×TBS with 0.1% [v/v] Tween 20) for at least 1 h at room temperature. The blots were probed with streptavidin-HRP (1:10000 dilution, Thermo Fisher N100) in 1×TBS with 0.1% [v/v] Tween 20 (10 mL) at room temperature for 1 h while rocking. To visualize cellular loading controls, membranes were probed with anti-GFP rabbit polyclonal antibody (1:1000 dilution in 5% BSA in TBST, ProteinTech 50430-2-AP) and anti-GAPDH rabbit polyclonal antibody (1:1000 dilution in 5% BSA inTBST, ProteinTech 10494-1-AP) at 4° C. overnight with rocking. Membranes were then probed with the secondary goat anti-rabbit polyclonal antibody (1:5000 dilution in TBST, Santa Cruz Biotechnology sc-2004) for 1 hour at room temperature with rocking. Chemiluminesence was developed using SuperSignal West Pico Chemiluminescent Substrate (Thermo Fisher, 8 mL) and imaged on a ChemiDoc Imaging System (BioRad) using automated exposure settings. To visualize in vitro loading controls, gels were developed with silver stain (Pierce, ThermoFisher 24162) according to manufacturer's protocol and imaged on ChemiDoc Imaging system using automated exposure settings. For quantification, bands were integrated using ImageJ by drawing rectangles around lanes and analyzing using the plot lanes function. Band integrations were normalized to the loading control.

### Plasmid Construction

pcDNA-PTK7-VSV-CFP was generated from PCR amplification of the CFP gene in pLyn-FKBP—FKBP-CFP using primers P1 and P2 and cloned into the pcDNA3-PTK7-VSV backbone, amplified using primers P3 and P4. PCR products were gel purified (Thermo Fisher, K0692) according to fragment length and annealed using published Gibson assembly method. Template DNA was digested using DpnI, then transformed into E. coli and miniprepped. Plasmid sequences were confirmed by Genewiz Sanger sequencing using BGHR reverse primer and EGFP-N forward primer (P5 and P6).

### Cell Culture Maintenance

All cell culture experiments were performed in a sterile laminar flow hood. HEK293T and COS7 cells were maintained in Dulbecco's Modified Eagle Medium (DMEM) supplemented with 10% (v/v) fetal bovine serum and 1% (v/v) penicillin/streptomycin at 37° C. with 5% CO2. Cells were used between passage number 6 and 30. Cell lines were tested for mycoplasma contamination every 6 months.

### PTK7 Labeling on Cells

Cells were seeded into a 48 well plate in 250 μL of DMEM (antibiotic free). When cells reached 80-90% confluency, each well was transfected with 400 ng of plasmid, using 0.8 μL of P3000 and 0.8 μL of Lipofectamine 3000, diluted in Opti-MEM transfection media to a total volume of 20 μL per well. Cells were incubated at 37° C. with 5% CO₂ overnight. After checking for CFP fluorescence, cells were washed three times with 200 μL of DPBS. Labeling was performed by incubating with 100 μL of the indicated concentration of sgc8c-27-(1) in DPBS with 0.1 mg/mL yeast tRNA for indicated time length at 37° C. Cells were then washed three more times with 100 μL of DPBS, lysed with 75 μL RIPA buffer containing 100×HALT protease inhibitor, and stored at −80° C. until further use.

### Streptavidin Pulldown

Biotinylated proteins were isolated from crude lysate by incubating with Streptavidin Sepharose bead slurry, prewashed with PBS, for 1 hour, followed by 3 washes of 100 μL PBS+0.1% SDS+1% NP-40, 2 washes of 100 μL PBS+0.4 M NaCL+0.1% SDS+1% NP-40, and 1 was of 100 μL PBS. Biotinylated protein was eluted by boiling for 15 minutes at 95° C. in 10 μL 6×Laemmli buffer with 8% (v/v) 2-mercapto ethanol with 10 μL 6 mM biotin.

### Live Cell Fluorescence Imaging

Cells were seeded into a 96 well plate in 100 μL of DMEM (antibiotic free). When cells reached₈₀-9₀% confluency, each well was transfected with 100 ng of plasmid, using 0.4 μL of P3000 and 0.4 μL of Lipofectamine 3000, diluted in Opti-MEM transfection media to a total volume of 10 μL per well. Cells were incubated at 37° C. with 5% CO₂ overnight. Media on cells was replaced with 100 μL fresh, anti-biotic free DMEM after 24 hours. After 36 hours total incubation after transfection, cells were washed three times with 100 μL of DPBS. Labeling was performed by incubating with 40 μL of the indicated concentration of sgc8c-27-(1) in DPBS with 0.1 mg/mL yeast tRNA for 1 h in DPBS, unless otherwise specified, at 37° C. Cells were washed three more times with 40 μL of DPBS and incubated with 1 μg/mL neutravidin-tetramethylrhodamine on ice for 10 minutes. Cells were washed three more times with 40 μL of DPBS. Cells were imaged in 40 μL of Live Cell Imaging Solution (Invitrogen) using a Zeiss Axio Observer Z1 with an Andor Zyla 4.2 camera, LD Plan-Neofluar 40×/0/6 objective, and the GFP (38 HE, ex. BP 470/40, em. BP 525/50) and dsRed (43 HE, ex. BP 550/25, em. BP 605/70) filter sets. Images were exported in TIFF format using ZEN pro (Zeiss) software package. Image processing was conducted using ImageJ software package. Labeling quantification was performed in ImageJ by selecting pixels expressing CFP as a gate. Tetra-methyl rhodamine fluorescence was normalized to CFP fluorescence.

### E. REFERENCES

A. D. Ellington, J. W. Szostak, Nature 1990, 346, 818-822.
A. Olszewska, R. Pohl, M. Brazdova, M. Fojta, M. Hocek, Bioconjug Chem 2016, 27, 2089-2094;
A. Panattoni, R. Pohl, M. Hocek, Org Lett 2018, 20, 3962-3965.
A. S. Wolberg, R. A. Campbell, Transfus Apher Sci 2008, 38, 15-23.
Avci-Adali, M.; Paul, A.; Wilhelm, N.; Ziemer, G.; Wendel, H. P., Upgrading SELEX technology by using lambda exonuclease digestion for single-stranded DNA generation. Molecules 2009, 15 (1), 1-11.
B. A. Williams, L. Lin, S. M. Lindsay, J. C. Chaput, J Am Chem Soc 2009, 131, 6330-6331.
B. F. Cravatt, A. T. Wright, J. W. Kozarich, Annu Rev Biochem 2008, 77, 383-414.
Birchmeier, C.; Birchmeier, W.; Gherardi, E.; Vande Woude, G. F., Met, metastasis, motility and more. Nat Rev Mol Cell Biol 2003, 4 (12), 915-25.
Boccaccio, C.; Comoglio, P. M., Invasive growth: a MET-driven genetic programme for cancer and stem cells. Nat Rev Cancer 2006, 6 (8), 637-45.
Bouattour, M.; Raymond, E.; Qin, S.; Cheng, A. L.; Stammberger, U.; Locatelli, G.; Faivre, S., Recent developments of c-Met as a therapeutic target in hepatocellular carcinoma. Hepatology 2018, 67 (3), 1132-1149.
C. Cui, H. Zhang, R. Wang, S. Cansiz, X. Pan, S. Wan, W. Hou, L. Li, M. Chen, Y. Liu, X. Chen, Q. Liu, W. Tan, Angew Chem Int Ed Engl 2017, 56, 11954-11957.
C. Negrier, M. Shima, M. Hoffman, Blood Rev 2019, 38, 100582.
C. Tuerk, L. Gold, science 1990, 249, 505-510;
Chaput, J. C., Redesigning the Genetic Polymers of Life. Acc Chem Res 2021, 54 (4), 1056-1065
Chen, R.; Khatri, P.; Mazur, P. K.; Polin, M.; Zheng, Y.; Vaka, D.; Hoang, C. D.; Shrager, J.; Xu, Y.; Vicent, S.; Butte, A. J.; Sweet-Cordero, E. A., A meta-analysis of lung cancer gene expression identifies PTK7 as a survival gene in lung adenocarcinoma. Cancer Res 2014, 74 (10), 2892-902.
D. C. Dehigaspitiya, S. Navath, C. S. Weber, R. M. Lynch, E. A. Mash, Tetrahedron Lett 2015, 56, 3060-3065.
Das, P. M.; Ramachandran, K.; vanWert, J.; Singal, R., Chromatin immunoprecipitation assay. Biotechniques 2004, 37 (6), 961-9.
Dunn, M. R.; McCloskey, C. M.; Buckley, P.; Rhea, K.; Chaput, J. C., Generating Biologically Stable TNA Aptamers that Function with High Affinity and Thermal Stability. J Am Chem Soc 2020, 142 (17), 7721-7724.
Ellington, A. D.; Szostak, J. W., In vitro selection of RNA molecules that bind specific ligands. Nature 1990, 346 (6287), 818-22.
F. N. Edfeldt, R. H. Folmer, A. L. Breeze, Drug Discov Today 2011, 16, 284-287.
G. L. Verdine, D. P. Norman, Annu Rev Biochem 2003, 72, 337-366;
G. Mayer, Angew Chem Int Ed Engl 2009, 48, 2672-2689;
Han, J.; Lee, H.; Nguyen, N. Y.; Beaucage, S. L.; Puri, R. K., Novel multiple 5′-amino-modified primer for DNA microarrays. Genomics 2005, 86 (2), 252-8.
Huang, Z.; Wang, D.; Long, C. Y.; Li, S. H.; Wang, X. Q.; Tan, W., Regulating the Anticancer Efficacy of Sgc8-Combretastatin A4 Conjugates: A Case of Recognizing the Significance of Linker Chemistry for the Design of Aptamer-Based Targeted Drug Delivery Strategies. J Am Chem Soc 2021, 143 (23), 8559-8564.
I. Ivancova, R. Pohl, M. Hubalek, M. Hocek, Angew Chem Int Ed Engl 2019, 58, 13345-13348.
I. Willner, M. Zayats, Angew Chem Int Ed Engl 2007, 46, 6408-6418;
J. A. Huntington, T. P. Baglin, Trends Pharmacol Sci 2003, 24, 589-595.
J. B. Trads, T. Torring, K. V. Gothelf, Acc Chem Res 2017, 50, 1367-1374;
J. Dadova, P. Orsag, R. Pohl, M. Brazdova, M. Fojta, M. Hocek, Angew Chem Int Ed Engl 2013, 52, 10515-10518;
J. J. van Veen, A. Gatt, M. Makris, Br J Haematol 2008, 142, 889-903.
J. Lotze, U. Reinhardt, O. Seitz, A. G. Beck-Sickinger, Mol Biosyst 2016, 12, 1731-1745.
J. M. Strelow, SLAS Discov 2017, 22, 3-20;
J. N. Rybak, S. B. Scheurer, D. Neri, G. Elia, Proteomics 2004, 4, 2296-2299.
J. Parkinson, Journal of the Chemical Society, Perkin Transactions 1 1998, 1131-1138.
J. Singh, R. C. Petter, A. F. Kluge, Curr Opin Chem Biol 2010, 14, 475-480;
J. Singh, R. C. Petter, T. A. Baillie, A. Whitty, Nat Rev Drug Discov 2011, 10, 307-317;
J. Weisner, R. Gontla, L. van der Westhuizen, S. Oeck, J. Ketzer, P. Janning, A. Richters, T. Muhlenberg, Z. Fang, A. Taher, V. Jendrossek, S. C. Pelly, S. Bauer, W. A. van Otterlo, D. Rauh, Angew Chem Int Ed Engl 2015, 54, 10313-10316;
J. Zhou, J. Rossi, Nat Rev Drug Discov 2017, 16, 440;
Jie, Y.; Liu, G.; Feng, L.; Li, Y.; E, M.; Wu, L.; Li, Y.; Rong, G.; Li, Y.; Wei, H.; Gu, A., PTK7-Targeting CAR T-Cells for the Treatment of Lung Cancer and Other Malignancies. Front Immunol 2021, 12, 665970.
K. Chen, B. Liu, B. Yu, W. Zhong, Y. Lu, J. Zhang, J. Liao, J. Liu, Y. Pu, L. Qiu, L. Zhang, H. Liu, W. Tan, Wiley Interdiscip Rev Nanomed Nanobiotechnol 2017, 9;
K. G. Mann, S. Butenas, K. Brummel, Arterioscler Thromb Vasc Biol 2003, 23, 17-25.
K. Lang, J. W. Chin, Chem Rev 2014, 114, 4764-4806.
K. M. Backus, B. E. Correia, K. M. Lum, S. Forli, B. D. Horning, G. E. Gonzalez-Paez, S. Chatterjee, B. R. Lanning, J. R. Teijaro, A. J. Olson, D. W. Wolan, B. F. Cravatt, Nature 2016, 534, 570-574;
K. Wakui, T. Yoshitomi, A. Yamaguchi, M. Tsuchida, S. Saito, M. Shibukawa, H. Furusho, K. Yoshimoto, Mol Ther Nucleic Acids 2019, 16, 348-359.
K. Wang, D. Fan, Y. Liu, E. Wang, Biosens Bioelectron 2015, 73, 1-6;
K.-M. Song, S. Lee, C. Ban, Sensors 2012, 12, 612-631;
Kim, C.; Ryu, D. K.; Lee, J.; Kim, Y. I.; Seo, J. M.; Kim, Y. G.; Jeong, J. H.; Kim, M.; Kim, J. I.; Kim, P.; Bae, J. S.; Shim, E. Y.; Lee, M. S.; Kim, M. S.; Noh, H.; Park, G. S.; Park, J. S.; Son, D.; An, Y.; Lee, J. N.; Kwon, K. S.; Lee, J Y.; Lee, H.; Yang, J. S.; Kim, K. C.; Kim, S. S.; Woo, H. M.; Kim, J. W.; Park, M. S.; Yu, K. M.; Kim, S. M.; Kim, E. H.; Park, S. J.; Jeong, S. T.; Yu, C. H.; Song, Y.; Gu, S. H.; Oh, H.; Koo, B. S.; Hong, J. J.; Ryu, C. M.; Park, W. B.; Oh, M. D.; Choi, Y. K.; Lee, S. Y., A therapeutic neutralizing antibody targeting receptor binding domain of SARS-CoV-2 spike protein. Nat Commun 2021, 12 (1), 288.
Kishi, K.; Sakai, H.; Seto, T.; Kozuki, T.; Nishio, M.; Imamura, F.; Nokihara, H.; Satouchi, M.; Nakagawa, S.; Tahata, T.; Nakagawa, K., First-line onartuzumab plus erlotinib treatment for patients with MET-positive and EGFR mutation-positive non-small-cell lung cancer. Cancer Treat Res Commun 2019, 18, 100113.
Koshi, Y.; Nakata, E.; Miyagawa, M.; Tsukiji, S.; Ogawa, T.; Hamachi, I., Target-specific chemical acylation of lectins by ligand-tethered DMAP catalysts. J Am Chem Soc 2008, 130 (1), 245-51.
Kroiher, M.; Miller, M. A.; Steele, R. E., Deceiving appearances: signaling by “dead” and “fractured” receptor protein-tyrosine kinases. Bioessays 2001, 23 (1), 69-76.
L. C. Bock, L. C. Griffin, J. A. Latham, E. H. Vermaas, J. J. Toole, Nature 1992, 355, 564-566.
L. D. Lavis, R. T. Raines, ACS Chem Biol 2008, 3, 142-155.
Langer, P. R.; Waldrop, A. A.; Ward, D. C., Enzymatic synthesis of biotin-labeled polynucleotides: novel nucleic acid affinity probes. Proc Natl Acad Sci USA 1981, 78 (11), 6633-7.
Lin, Y.; Zhang, L. H.; Wang, X. H.; Xing, X. F.; Cheng, X. J.; Dong, B.; Hu, Y.; Du, H.; Li, Y. A.; Zhu, Y. B.; Ding, N.; Du, Y. X.; Li, J. Y.; Ji, J. F., PTK7 as a novel marker for favorable gastric cancer patient survival. J Surg Oncol 2012, 106 (7), 880-6.
Lu, X.; Borchers, A. G.; Jolicoeur, C.; Rayburn, H.; Baker, J. C.; Tessier-Lavigne, M., PTK7/CCK-4 is a novel regulator of planar cell polarity in vertebrates. Nature 2004, 430 (6995), 93-8.
M. B. Skovsgaard, M. R. Mortensen, J. Palmfeldt, K. V. Gothelf, Bioconjug Chem 2019, 30, 2127-2135;
M. Famulok, G. Mayer, Acc Chem Res 2011, 44, 1349-1358; M. Gehringer, S. A. Laufer, J Med Chem 2019, 62, 5673-5724; Maitland, M. L.; Sachdev, J. C.; Sharma, M. R.; Moreno, V.; Boni, V.; Kummar, S.; Stringer-Reasor, E.; Lakhani, N.; Moreau, A. R.; Xuan, D.; Li, R.; Powell, E. L.; Jackson-Fisher, A.; Bowers, M.; Alekar, S.; Xin, X.; Tolcher, A. W.; Calvo, E., First-in-Human Study of PF-06647020 (Cofetuzumab Pelidotin), an Antibody-Drug Conjugate Targeting Protein Tyrosine Kinase 7, in Advanced Solid Tumors. Clin Cancer Res 2021, 27 (16), 4511-4520.
Mencin, N.; Smuc, T.; Vranicar, M.; Mavri, J.; Hren, M.; Galesa, K.; Krkoc, P.; Ulrich, H.; Solar, B., Optimization of SELEX: comparison of different methods for monitoring the progress of in vitro selection of aptamers. J Pharm Biomed Anal 2014, 91, 151-9.
Mossie, K.; Jallal, B.; Alves, F.; Sures, I.; Plowman, G. D.; Ullrich, A., Colon carcinoma kinase-4 defines a new subclass of the receptor tyrosine kinase family. Oncogene 1995, 11 (10), 2179-84
N. K. Navani, Y. Li, Curr Opin Chem Biol 2006, 10, 272-281;
Ou, X.; Liu, Y.; Lei, X.; Li, P.; Mi, D.; Ren, L.; Guo, L.; Guo, R.; Chen, T.; Hu, J.; Xiang, Z.; Mu, Z.; Chen, X.; Chen, J.; Hu, K.; Jin, Q.; Wang, J.; Qian, Z., Characterization of spike glycoprotein of SARS-CoV-2 on virus entry and its immune cross-reactivity with SARS-CoV. Nat Commun 2020, 11 (1), 1620.
Park, S.-K.; Lee, H.-S.; Lee, S.-T., Characterization of the Human Full-Length PTK7 cDNA Encoding a Receptor Protein Tyrosine Kinase-Like Molecule Closely Related to Chick KLG1. The Journal of Biochemistry 1996, 119 (2), 235-239.
R. J. Beynon, J. M. Pratt, Mol Cell Proteomics 2005, 4, 857-872.
R. Mah, J. R. Thomas, C. M. Shafer, Bioorg Med Chem Lett 2014, 24, 33-39;
R. Micura, C. Hobartner, Chem Soc Rev 2020, 49, 7331-7353;
R. R. White, B. A. Sullenger, C. P. Rusconi, J Clin Invest 2000, 106, 929-934.
Roxo, C.; Kotkowiak, W.; Pasternak, A., G-Quadruplex-Forming Aptamers-Characteristics, Applications, and Perspectives. Molecules 2019, 24 (20).
S. A. Ingale, H. Mei, P. Leonard, F. Seela, J Org Chem 2013, 78, 11271-11282.
S. K. Silverman, Trends Biochem Sci 2016, 41, 595-609.
S. Lopez-Gomollon, F. E. Nicolas, Methods Enzymol 2013, 529, 65-83.
S. M. Douglas, I. Bachelet, G. M. Church, Science 2012, 335, 831-834;
S. M. Nimjee, R. R. White, R. C. Becker, B. A. Sullenger, Annu Rev Pharmacol Toxicol 2017, 57, 61-79;
S. P. Ryder, M. I Recht, J. R. Williamson, Methods Mol Biol 2008, 488, 99-115.
S. Romero-Perez, I. Lopez-Martin, M. C. Martos-Maldonado, A. Somoza, D. Gonzalez-Rodriguez, Org Lett 2020, 22, 41-45.
S. Tsukiji, M. Miyagawa, Y. Takaoka, T. Tamura, I. Hamachi, Nat Chem Biol 2009, 5, 341-343.
Seela, F.; Sirivolu, V. R.; Chittepu, P., Modification of DNA with octadiynyl side chains: synthesis, base pairing, and formation of fluorescent coumarin dye conjugates of four nucleobases by the alkyne—azide “click” reaction. Bioconjug Chem 2008, 19 (1), 211-24.
Sefah, K.; Shangguan, D.; Xiong, X.; O'Donoghue, M. B.; Tan, W., Development of DNA aptamers using Cell-SELEX. Nat Protoc 2010, 5 (6), 1169-85.
Shangguan, D.; Cao, Z.; Meng, L.; Mallikaratchy, P.; Sefah, K.; Wang, H.; Li, Y.; Tan, W., Cell-specific aptamer probes for membrane protein elucidation in cancer cells. J Proteome Res 2008, 7 (5), 2133-9.
Sicco, E.; Monaco, A.; Fernandez, M.; Moreno, M.; Calzada, V.; Cerecetto, H., Metastatic and non-metastatic melanoma imaging using Sgc8-c aptamer PTK7-recognizer. Sci Rep 2021, 11 (1), 19942.
Song, Y.; Song, J.; Wei, X.; Huang, M.; Sun, M.; Zhu, L.; Lin, B.; Shen, H.; Zhu, Z.; Yang, C., Discovery of Aptamers Targeting the Receptor-Binding Domain of the SARS-CoV-2 Spike Glycoprotein. Anal Chem 2020, 92 (14), 9895-9900.
Stoltenburg, R.; Reinemann, C.; Strehlitz, B., SELEX—a (r)evolutionary method to generate high-affinity nucleic acid ligands. Biomol Eng 2007, 24 (4), 381-403.
Sun, J. J.; Li, H. L.; Guo, S. J.; Ma, H.; Liu, S. J.; Liu, D.; Xue, F. X., The Increased PTK7 Expression Is a Malignant Factor in Cervical Cancer. Dis Markers 2019, 2019, 5380197.
T. A. Baillie, Angew Chem Int Ed Engl 2016, 55, 13408-13421.
T. Tamura, I. Hamachi, J Am Chem Soc 2018, 141, 2782-2799.
T. Tamura, T. Ueda, T. Goto, T. Tsukidate, Y. Shapira, Y. Nishikawa, A. Fujisawa, I. Hamachi, Nat Commun 2018, 9, 1870.
T. Tamura, T. Ueda, T. Goto, T. Tsukidate, Y. Shapira, Y. Nishikawa, A. Fujisawa, I. Hamachi, Nat Commun 2018, 9, 1870.
T. Zhang, J. M. Hatcher, M. Teng, N. S. Gray, M. Kostic, Cell Chem Biol 2019, 26, 1486-1500;
Tamura, T.; Song, Z.; Amaike, K.; Lee, S.; Yin, S.; Kiyonaka, S.; Hamachi, I., Affinity-Guided Oxime Chemistry for Selective Protein Acylation in Live Tissue Systems. J Am Chem Soc 2017, 139 (40), 14181-14191.
Tuerk, C.; Gold, L., Systematic evolution of ligands by exponential enrichment: RNA ligands to bacteriophage T4 DNA polymerase. Science 1990, 249 (4968), 505-10.
Ueki, R.; Sando, S., A DNA aptamer to c-Met inhibits cancer cell migration. Chem Commun (Camb) 2014, 50 (86), 13131-4.
van der Geer, P.; Hunter, T.; Lindberg, R. A., Receptor protein-tyrosine kinases and their signal transduction pathways. Annu Rev Cell Biol 1994, 10, 251-337.
Y. Takaoka, Y. Sun, S. Tsukiji, I. Hamachi, Chem Sci J2011, 2, 511-520.
Y. Zhang, D. Duan, Y. Zhong, X. A. Guo, J. Guo, J. Gou, Z. Gao, B. Yu, Org Lett 2019, 21, 4960-4965.
Z. Cao, R. Tong, A. Mishra, W. Xu, G. C. Wong, J. Cheng, Y. Lu, Angew Chem Int Ed Engl 2009, 48, 6494-6498.
Z. Zhao, J. Fu, S. Dhakal, A. Johnson-Buck, M. Liu, T. Zhang, N. W. Woodbury, Y. Liu, N. G. Walter, H. Yan, Nat Commun 2016, 7, 10619; i) J. L. Counihan, E. A. Grossman, D. K. Nomura, Chem Rev 2018, 118, 6893-6923; j) A. Cuesta, J. Taunton, Annu Rev Biochem 2019, 88, 365-381
Zhang, Y.; Gao, H.; Zhou, W.; Sun, S.; Zeng, Y.; Zhang, H.; Liang, L.; Xiao, X.; Song, J.; Ye, M.; Yang, Y.; Zhao, J.; Wang, Z.; Liu, J., Targeting c-met receptor tyrosine kinase by the DNA aptamer SL1 as a potential novel therapeutic option for myeloma. J Cell Mol Med 2018, 22 (12), 5978-5990.

What is claimed is:

1. A modified aptamer-handle conjugate comprising an aptamer, an electrophilic leaving group and a handle.

2. A modified aptamer comprising an electrophilic leaving group for crosslinking to a target.

3. The modified aptamer-handle conjugate of claim 1 or 2, wherein the aptamer comprises any of the sequences as set forth in SEQ ID NO: 1, SEQ ID NO: 2, SEQ ID NO: 3, SEQ ID NO: 4, SEQ ID NO: 5, SEQ ID NO: 6, or SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, and/or SEQ ID NO: 20.

4. The modified aptamer or modified aptamer-handle conjugate of any of claims 1-3, wherein the electrophilic group comprises N-acyl sulfonamide cleavable electrophile or tosyl cleavable electrophile.

5. The modified aptamer or modified aptamer-handle conjugate of any of claims 1-4, wherein the electrophile is bound to the aptamer via the aryl sulfonamide of the electrophile.

6. The modified aptamer or modified aptamer-handle conjugate of any of claims 1-5, wherein the electrophile is bound to the aptamer via the amide motif of the electrophile.

7. The modified aptamer or modified aptamer-handle conjugate of any of claims 1-5, wherein the electrophile comprises N-acyl sulfonamide; and wherein the N-acyl sulfonamides is connected to the aptamer via the carbonyl group of the cleavable amide.

8. The modified aptamer or modified aptamer-handle conjugate of any of claims 1-5, wherein the electrophile comprises N-acyl sulfonamide; and wherein the N-acyl sulfonamide is connected to the aptamer via the non-cleavable benzamide nitrogen.

9. The modified aptamer or modified aptamer-handle conjugate of any of claims 1-5, wherein the electrophile comprises N-acyl sulfonamide; and wherein the N-acyl sulfonamide is connected to the aptamer via any non-cleavable connection to the aryl ring of the sulfonamide group.

10. The modified aptamer or modified aptamer-handle conjugate of any of claims 1-5, wherein the electrophile comprises a tosyl electrophile; and wherein the tosyl electrophile is connected to the aptamer via the non-cleavable benzamide nitrogen.

11. The modified aptamer or modified aptamer-handle conjugate of any of claims 1-5, wherein the electrophile comprises a tosyl electrophile; and wherein the tosyl electrophile is connected to the aptamer via any non-cleavable connection to the aryl ring of the tosylate group.

12. The modified aptamer or modified aptamer-handle conjugate of any of claims 1-5, wherein the electrophile comprises a tosyl electrophile; and wherein the tosyl electrophile is connected to the aptamer via the alpha-CH₂ carbon of the cleavable tosylate moiety.

13. The modified aptamer or modified aptamer-handle conjugate of any of claims 1-12, wherein the handle comprises biotin, bioconjugation handle, a chemiluminescent marker, a fluorescent marker, radiomarker, dye, quantum dot, enzyme, enzyme substrate, catalyst, small molecule ligand, drug, PROTAC® (E3 ligase ligand), or LYTAC® (cation-independent mannose-6-phosphate receptor (CI-M6PR) ligand).

14. A pharmaceutical composition comprising a therapeutically effective amount of the modified aptamer or modified aptamer-handle conjugate of any of claims 1-13 and a pharmaceutical carrier.

15. A method of detecting a coronavirus infection comprising diagnosing and/or detecting a coronavirus infection comprising a) contacting a tissue sample or cell sample with the modified aptamer handle conjugate of any of claims 1-13, wherein the aptamer selectively binds the corona virus spike protein or the receptor binding domain of the coronavirus spike or nucleocapsid protein; (b) measuring the presence, absence or quantity of the aptamer; and (c) diagnosing and/or detecting a coronavirus infection based on the presence, absence or quantity of the aptamer measured.

16. A method of treating a coronavirus infection comprising administering to a subject with a coronavirus infection the modified aptamer or the modified aptamer handle complex of any of claims 1-13 or pharmaceutical composition of claim 14, wherein the aptamer selectively binds the coronavirus spike protein or the receptor binding domain of the coronavirus spike protein.

17. The method of detecting a coronavirus infection of claim 15 or treating a coronavirus infection of claim 16, wherein the aptamer comprises the sequence set forth in SEQ ID NO: 9, SEQ ID NO: 10, SEQ ID NO: 11, SEQ ID NO: 12, SEQ ID NO: 13, SEQ ID NO: 14, and/or SEQ ID NO: 15.

18. The method of detecting and/or diagnosing a coronavirus infection or treating a coronavirus infection of any of claims 15-17; wherein the coronavirus comprises avian coronavirus (IBV), porcine coronavirus HKU15 (PorCoV HKU15), porcine epidemic diarrhea virus (PEDV), HCoV-229E, HCoV-OC43, HCoV-HKU1, HCoV-NL63, SARS-CoV, SARS-CoV-2, or MERS-CoV.

19. The method of detecting a coronavirus infection of claim 18, wherein the coronavirus comprises SARS-CoV-2.

20. A method of detecting a circulatory condition in a subject comprising a) contacting a tissue sample or cell sample with the modified aptamer handle conjugate of any of claims 1-13; (b) measuring the presence, absence or quantity of the aptamer; and (c) diagnosing and/or detecting circulatory condition based on the presence, absence or quantity of the aptamer measured in the tissue sample of cell sample.

21. A method of treating circulatory condition in a subject comprising administering to the subject the modified aptamer or the modified aptamer handle conjugate of any of claims 1-13 or the pharmaceutical composition of claim 14.

22. The method of detecting and/or diagnosing a circulatory condition of claim 20 and/or treating a circulatory condition of claim 21; wherein the circulatory condition comprises thrombosis, thromboembolism, Paget-Schroetter disease, fibrosis, stroke, or myocardial infarction.

23. The method of detecting and/or diagnosing a circulatory condition of claim or method of treating a circulatory condition of any of claims 20-22, wherein the aptamer selectively binds to the thrombin protein.

24. The method of detecting a circulatory condition or method of treating a circulatory condition of any of claims 20-23, wherein the aptamer comprise the sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8.

25. A method of detecting and/or diagnosing a cancer and/or metastasis in a subject comprising contacting a tissue sample or cell sample with the modified aptamer handle complex of any of claims 1-13; (b) measuring the presence, absence or quantity of the aptamer; and (c) diagnosing and/or detecting the disease state of the tissue or cell based on the presence, absence or quantity of the aptamer measured.

26. A method of treating a cancer in a subject comprising administering to the subject the modified aptamer or the modified aptamer handle complex of any of claims 1-13 or pharmaceutical composition of claim 14.

27. The method of detecting and/or diagnosis a cancer and/or metastasis of treating a cancer and/or metastasis of any of claims 23-26, wherein the cancer comprises retinoblastoma, colon, lung, and gastric cancers.

28. The method of detecting and/or diagnosis a cancer and/or metastasis of treating a cancer and/or metastasis of any of claims 23-27, wherein the aptamer comprises the sequence as set forth in SEQ ID NO: 16 or SEQ ID NO: 17.

29. A method of delivering biotin, a chemiluminescent marker, a fluorescent marker, radiomarker, dye, quantum dot, enzyme, enzyme substrate, catalyst, small molecule ligand, PROTAC® (E3 ligase ligand), or LYTAC® (cation-independent mannose-6-phosphate receptor (CI-M6PR) ligand) a or to a target cell comprising conjugating a labeled electrophile to an aptamer creating a modified aptamer or an aptamer-handle complex; and contacting the target cell with said aptamer-handle complex.

30. A method of labeling one or more protein targets on or in a cell comprising conjugating a labeled electrophile to an aptamer creating a modified aptamer or an aptamer-handle complex; and contacting the cell with the aptamer electrophile complex.

31. The method of labeling one or protein targets on a cell of claim 30, wherein the protein target is an intracellular protein.

32. The method of labeling one or protein targets on a cell of claim 30, wherein the protein target is expressed on the cell membrane.

33. A modified aptamer-handle conjugate of Formula I 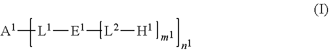
wherein:
A¹ comprises an aptamer;
L¹ and L² are independently selected from a bond or a linker;
E¹ is a cleavable electrophilic moiety;
H¹ is a handle;
m¹ is at least 1; and
n¹ is at least 1.

34. The modified aptamer-handle conjugate of claim 33, wherein E¹ is selected from a tosylate moiety, an acyl imidazole moiety, an electrophilic phenyl benzoate moiety, a N- or O-sulfonyl pyridine moiety, or an N-acyl sulfonamide moiety.

35. The modified aptamer-handle conjugate of claim 33, wherein E¹ is selected from 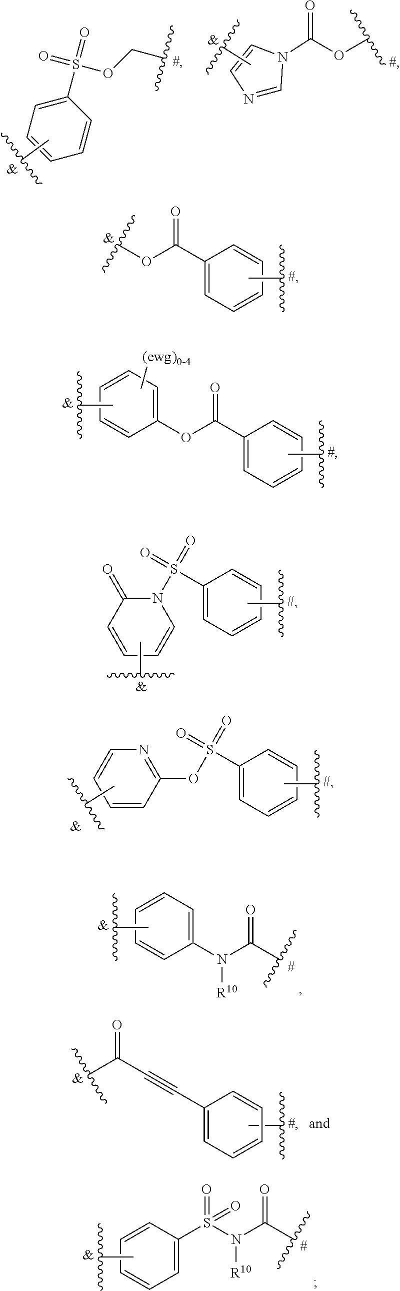
wherein:
one of & and # is the point of attachment to L¹ and the other of & and # is the point of attachment to L²;
ewg is independently at each occurrence an electron withdrawing group; and
R¹⁰ is selected from hydrogen, alkyl, cycloalkyl, heterocyclyl, aryl, or heteroaryl, wherein R¹⁰ may be optionally substituted as allowed by valency; and wherein each E¹ may be optionally substituted with one or more substituents as allowed by valency.

36. The modified aptamer-handle conjugate of claim 33, wherein E¹ is selected from: 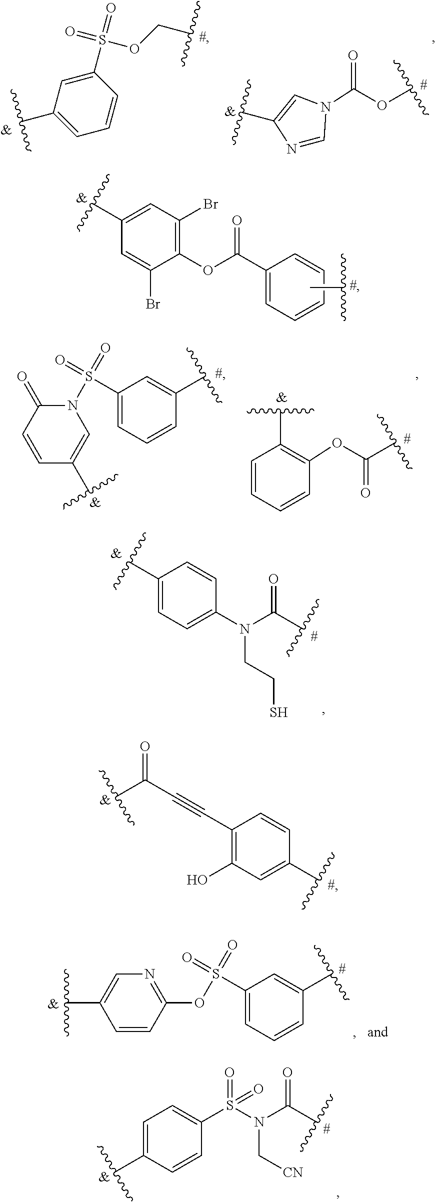
wherein one of & and # is the point of attachment to L¹ and the other of & and # is the point of attachment to L².

37. The modified aptamer-handle conjugate of claim 36, wherein E¹ is a moiety substituted with a group selected from

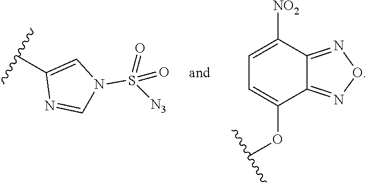 [SMILES: *c1cn(S(=O)(=O)N=[N+]=[N-])cn1 ; "and" ; *Oc1ccc([N+](=O)[O-])c2nonc12]

38. The modified aptamer-handle conjugate of any one of claims 33-37, wherein H¹ comprises biotin, a chemiluminescent marker, a fluorescent marker, radiomarker, dye, quantum dot, enzyme, enzyme substrate, catalyst, small molecule ligand, PROTAC® (E3 ligase ligand), or LYTAC® (cation-independent mannose-6-phosphate receptor (CI-M6PR) ligand).

39. The modified aptamer-handle conjugate of any one of claims 33-38, wherein L¹ comprises one or more ethylene glycol, propylene glycol, lactic and/or glycolic acid units.

40. The modified aptamer-handle conjugate of any one of claims 33-38, wherein L¹ is selected from L1 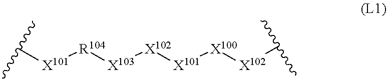
wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂ (alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

41. The modified aptamer-handle conjugate of any one of claims 33-40, wherein L² comprises one or more ethylene glycol, propylene glycol, lactic and/or glycolic acid units.

42. The modified aptamer-handle conjugate of any one of claims 33-40, wherein L² is selected from L1 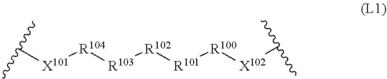
wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂ (alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

43. The modified aptamer-handle conjugate of any one of claims 33-42, wherein A¹ comprises an aptamer as set for in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

44. A method of labeling a protein or peptide target with a moiety comprising a handle H¹, the method comprising contacting the protein or peptide target with a modified aptamer-handle conjugate of any one of claims 33-43, wherein the aptamer A¹ of the modified aptamer-handle conjugate is capable of binding to the protein or peptide target.

45. A modified aptamer of Formula II 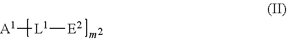
wherein:
A¹ comprises an aptamer;
L¹ is selected from a bond or a linker;
E² is an electrophilic moiety; and
m² is at least 1.

46. The modified aptamer of claim 45, wherein E² is selected from iodoacetamide, bromoacetamide, chloroacetamide, pentafluorophenylsulfonamides, 2-bromo-N-(prop-2-yn-1-yl)benzo[d]thiazole-6-carboxamide, 2-(methylsulfonyl)benzo[d]thiazole-6-carboxamide, 2-(methylsulfonyl)-4-phenyl-1,3,4-oxadiazole, 5-methyl-1,2-benziodoxol-3 (1H)-one, oxirane, maleimide, aryl and alkyl propiolamide, aryl and alkyl acrylamide, aryl and alkyl vinylsulfonamide, 2-fluoro-N-(hex-5-yn-1-yl)acrylamide, 2-thioxothiazolidine, N-acyl sulfonamide, 4-methoxycyclobut-3-ene-1,2-dione, 3-methoxy-4-(phenylamino)cyclobut-3-ene-1,2-dione, 5-(prop-2-yn-1-yloxy)benzaldehyde, 4-(hydroxymethyl)-3-nitro-benzamide, 1H-1,2,3-triazole-4-carbaldehyde, 3-phenyl-2H-azirine, (Z)—N-phenylacetohydrazonoyl chloride, benzenesulfonyl fluoride, 3H-1,2,4-triazole-3,5 (4H)-dione, benzenediazonium tetrafluoroborate, (3-phenyl-1,2-oxaziridin-2-yl)(4-piperidin-1-yl)methanone, 2,4,6-trimethyl-1-(methylcarbonylamino)pyridin-1-ium tetrafluoroborate, phosphorodichloridothioate, 3-(hydroxymethyl)naphthalen-2-ol, 4-hydroxy-3-(hydroxymethyl)benzenesulfonamide, 4-hydroxy-3-(methoxymethyl)benzenesulfonamide, and 2,2-dihydroxy-1-phenyl)ethan-1-one.

47. The modified aptamer of claim 45, wherein E² is selected from

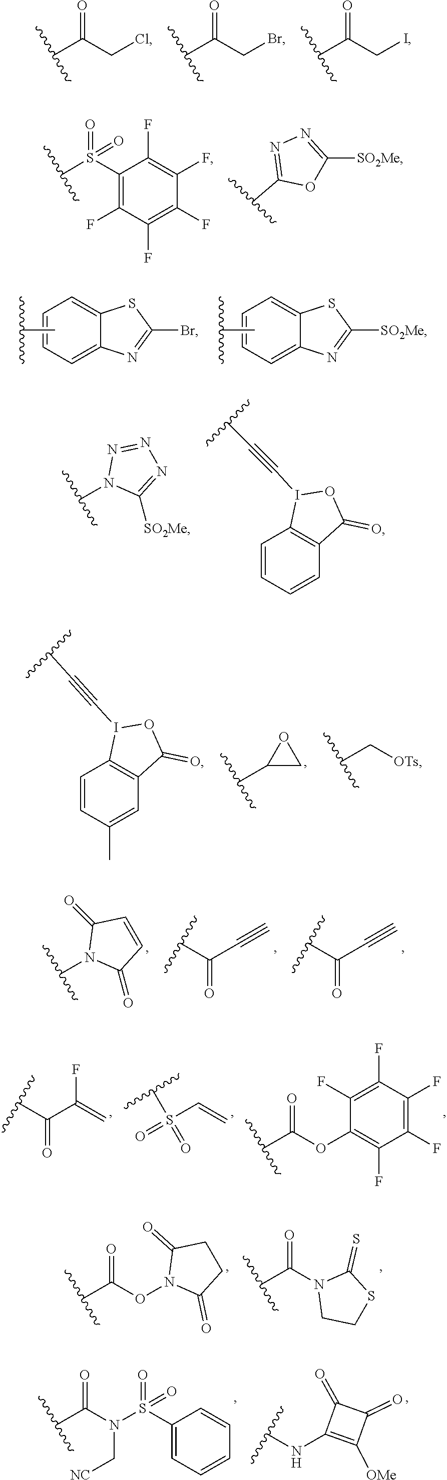

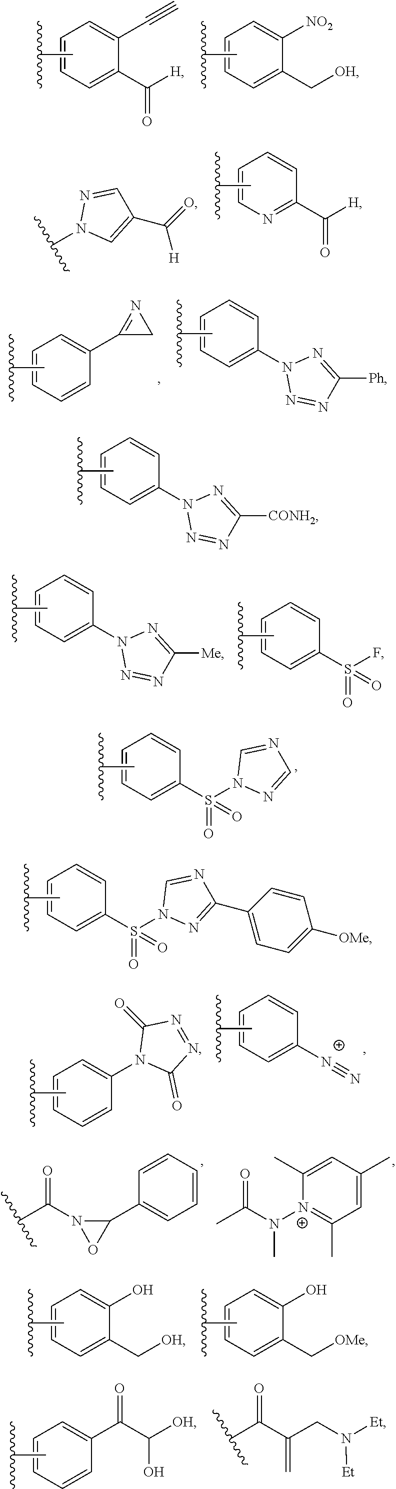

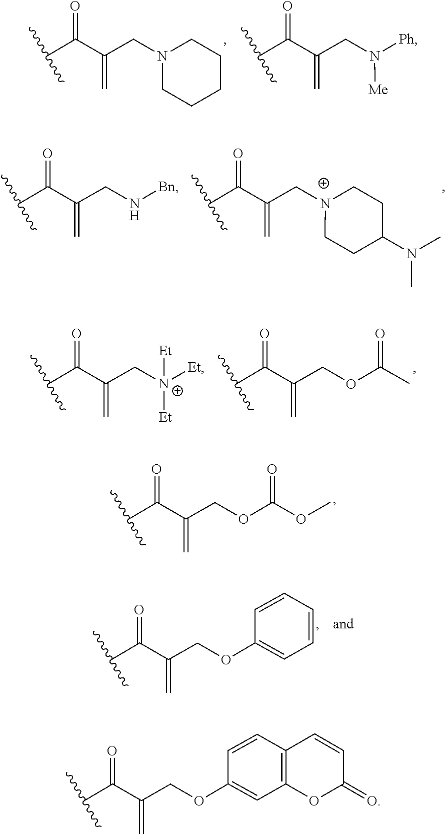

48. The modified aptamer of any one of claims 45-47, wherein L¹ comprises one or more ethylene glycol, propylene glycol, lactic and/or glycolic acid units.

49. The modified aptamer of any one of claims 45-47, wherein L¹ is selected from L1 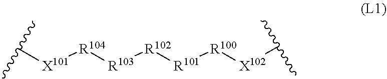
wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂ (alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

50. The modified aptamer-handle conjugate of any one of claims 45-49, wherein A¹ comprises an aptamer as set for in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

51. A method of labeling a protein or peptide target with a moiety comprising an handle A¹, the method comprising contacting the protein or peptide target with a modified aptamer-handle conjugate of any one of claims 45-50, wherein the aptamer A¹ of the modified aptamer is capable of binding to the protein or peptide target.

52. A modified aptamer of Formula III 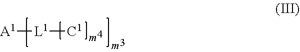
wherein:
A¹ comprises an aptamer;
L¹ is selected from a bond or a linker;
C¹ is a catalytic moiety;
m³ is at least 1; and
m⁴ is at least 1.

53. The modified aptamer of claim 52, wherein the modified aptamer of Formula III is capable of catalyzing the reaction of a compound of Formula IV 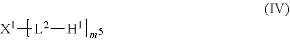
with a target, such that the target is substituted with a moiety comprising the H¹ group,
wherein X¹ is a moiety which converts into a reactive group capable of forming a covalent bond with the target upon contact with the modified aptamer of Formula III;
L² is selected from a bond or a linker;
H¹ is a handle; and
M⁵ is at least 1.

54. The modified aptamer of claim 52 or 53, wherein L¹ comprises one or more ethylene glycol, propylene glycol, lactic and/or glycolic acid units.

55. The modified aptamer of claim 52 or 53, wherein L¹ is selected from L1 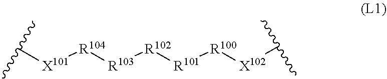
wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴¹ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂ (alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

56. The modified aptamer-handle conjugate of any one of claims 52-55, wherein A¹ comprises an aptamer as set for in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

57. A method of labeling a protein or peptide target with a moiety comprising a handle H¹, the method comprising contacting the protein or peptide target with a modified aptamer of any one of claims 52-56 in the presence of a compound of Formula IV 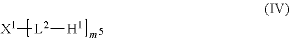
wherein X¹ is a moiety which converts into a reactive group capable of forming a covalent bond with the target upon contact with the modified aptamer;
L² is selected from a bond or a linker;
H¹ is a handle; and
m⁵ is at least 1; and
wherein the aptamer A¹ of the modified aptamer is capable of binding to the protein or peptide target.

58. A method for identifying aptamers suitable as ligands for a target from a candidate mixture of modified aptamers, the method comprising:
a) preparing a candidate mixture of modified aptamers, wherein each modified aptamer may independently be selected from a modified aptamer of Formula II-a 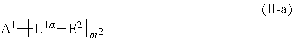
wherein:
A¹ comprises an aptamer;
L^{1A} is a linker comprising at least one cleavable moiety;
E² is an electrophilic moiety; and
m² is at least 1;
b) contacting the candidate mixture of modified aptamers with the target, wherein modified aptamers capable of forming a covalent bond with the target provide a subpopulation of covalently bound aptamers;
c) partitioning the subpopulation of covalently bound aptamers from a remainder of the candidate mixture of modified aptamers;
d) contacting the subpopulation of covalently bound aptamers with a reagent capable of reacting with the at least one cleavable moiety, wherein the at least one cleavable moiety is cleaved to provide a subpopulation of modified aptamers; and
e) amplifying the subpopulation of modified aptamers such that aptamers suitable as ligands for the target may be identified.

59. The method of claim 58, wherein steps a) to e) are repeated on the subpopulation of modified aptamers until an enriched mixture of modified aptamers suitable as ligands for the target is obtained.

60. A method for identifying aptamers suitable as ligands for a target from a candidate mixture of modified aptamers, the method comprising:
a) preparing a candidate mixture of modified aptamers, wherein each modified aptamer may independently be selected from a modified aptamer of Formula II 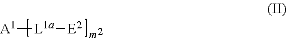
wherein:
A¹ comprises an aptamer;
L¹ is a bond or a linker;
E² is an electrophilic moiety; and
m² is at least 1;
b) contacting the candidate mixture of modified aptamers with the target, wherein modified aptamers capable of forming a covalent bond with the target provide a subpopulation of covalently bound aptamers;
c) partitioning the subpopulation of covalently bound aptamers from a remainder of the candidate mixture of modified aptamers;
d) contacting the subpopulation of covalently bound aptamers with a reagent capable of cleaving the target bound to the aptamer; and
e) amplifying the subpopulation of modified aptamers such that aptamers suitable as ligands for the target may be identified.

61. The method of claim 60, wherein steps a) to e) are repeated on the subpopulation of modified aptamers until an enriched mixture of modified aptamers suitable as ligands for the target is obtained.

62. The method of claim 60 or 61, wherein the target is a protein, and the reagent cleaves the target by degrading the protein.

63. A method of detecting the presence or quantifying the amount of a target protein comprising contacting the target protein with the modified aptamer-handle conjugate of any of the claims 1-13, wherein the aptamer selectively labels the target protein.

64. A method of detecting the presence or quantifying the amount of a target protein comprising contacting the target protein with a modified aptamer-handle conjugate comprising an aptamer, an electrophilic leaving group, and a handle; wherein the handle comprises or can be modified with a detectable label.

65. The method of claim 64, wherein the detectable label comprises biotin, a bioconjugation handle, a chemiluminescent marker, a fluorescent marker, a radiomarker, a dye, a quantum dot, an enzyme, an enzyme substrate, a catalyst, or a small molecule ligand.

66. The method of claim 63 or 64, further comprising measuring the presence, absence or quantity of the bound aptamer relative to a control or standard.

67. The method of detecting the presence of or quantifying the amount of a target protein of any of claims 63-65, further comprising transferring the target protein to a membrane after contacting the target protein with the aptamer-handle conjugate.

68. The method of detecting the presence of or quantifying the amount of a target protein of any of claims 63-65, wherein the handle comprises chemiluminescent marker.


SEQUENCE LISTING (22 sequences)

SEQ ID NO: 1 - DNA, 15 bp, Artificial Sequence
  Synthetic construct
ggttggtgtggttgg

SEQ ID NO: 2 - DNA, 15 bp, Artificial Sequence
  Synthetic construct
ggttggtgtggttgg

SEQ ID NO: 3 - DNA, 29 bp, Artificial Sequence
  Synthetic construct
agtccgtggtagggcaggttggggtgact

SEQ ID NO: 4 - RNA, 40 nt, Artificial Sequence
  Synthetic construct
gaacaaagcugaaguacuuacccaagaucaucccgaacga

SEQ ID NO: 5 - DNA, 26 bp, Artificial Sequence
  Synthetic construct
cgcctaggttgggtagggtggtggcg

SEQ ID NO: 6 - DNA, 32 bp, Artificial Sequence
  Synthetic construct
gtgacgtaggttggtgtggttggggcgtcacc

SEQ ID NO: 7 - DNA, 31 bp, Artificial Sequence
  Synthetic construct
ggttggtgtggttggtggttggtgtggttgg

SEQ ID NO: 8 - DNA, 70 bp, Artificial Sequence
  Synthetic construct
agcagcacagaggtcagatgatggggatggggggttggaggaatggatgacctatgcgtgctaccgtgaa

SEQ ID NO: 9 - DNA, 51 bp, Artificial Sequence
  Synthetic construct
cagcaccgaccttgtgctttgggagtgctggtccaagggcgttaatggaca

SEQ ID NO: 10 - DNA, 67 bp, Artificial Sequence
  Synthetic construct
atccagagtgacgcagcatttcatcgggtccaaaaggggctgctcgggattgcggatatggacacgt

SEQ ID NO: 11 - DNA, 86 bp, Artificial Sequence
  Synthetic construct
gggagaggagggagatagatatcaacccatggtaggtattgcttggtagggatagtgggcttgatgtttcgtggatgccacaggac

SEQ ID NO: 12 - DNA, 86 bp, Artificial Sequence; identical to SEQ ID NO: 11
  Synthetic construct

SEQ ID NO: 13 - DNA, 88 bp, Artificial Sequence
  Synthetic construct
gcaatggtacggtacttccggatgcggaaactggctaattggtgaggctggggcggtcgtgcagcaaaagtgcacgctactttgctaa

SEQ ID NO: 14 - DNA, 78 bp, Artificial Sequence
  Synthetic construct
gcaatggtacggtacttccggatgcggaaactggctaattggtgaggctggggcggtcgtgcagcaaaagtgcacgct

SEQ ID NO: 15 - DNA, 57 bp, Artificial Sequence
  Synthetic construct
gcaatggtacggtacttccggatgcggaaactggctaattggtgaggctggggcggt

SEQ ID NO: 16 - DNA, 41 bp, Artificial Sequence
  Synthetic construct
atctaactgctgcgccgccgggaaaatactgtacggttaga

SEQ ID NO: 17 - DNA, 88 bp, Artificial Sequence
  Synthetic construct
ataccagcttattcaattagtcacacttagagttctaactgctgcgccgccgggaaaatactgtacggttagatagtaagtgcaatct

SEQ ID NO: 18 - DNA, 60 bp, Artificial Sequence
  Synthetic construct
taatacgactcactatagggaccgaaaaagacctggcttctatactaagtctacgttccc

SEQ ID NO: 19 - DNA, 42 bp, Artificial Sequence
  Synthetic construct
tcacttcacaccgcatctctacgtccggttgcgctttccttt

SEQ ID NO: 20 - RNA, 23 nt, Artificial Sequence
  Synthetic construct
ggaggugcuccgaaaggaacucc

SEQ ID NO: 21 - DNA, 86 bp, Artificial Sequence
  Synthetic construct
  misc_feature (23)..(24): T is ethynyl dU (S1 or EdU) nucleotide
tagggaagagaaggacatatgatggttggtgtggttggtttttttttttttttttttttttttttgactagtacatgaccacttga

SEQ ID NO: 22 - DNA, 87 bp, Artificial Sequence
  Synthetic construct
  misc_feature (24)..(25): N is any nucleotide
  misc_feature (25)..(26): N is any nucleotide
  misc_feature (26)..(27): N is any nucleotide
  misc_feature (27)..(28): N is any nucleotide
  misc_feature (28)..(29): N is any nucleotide
  misc_feature (29)..(30): N is any nucleotide
  misc_feature (30)..(31): N is any nucleotide
  misc_feature (31)..(32): N is any nucleotide
  misc_feature (32)..(33): N is any nucleotide
  misc_feature (33)..(34): N is any nucleotide
  misc_feature (34)..(35): N is any nucleotide
  misc_feature (35)..(36): N is any nucleotide
  misc_feature (36)..(37): N is any nucleotide
  misc_feature (37)..(38): N is any nucleotide
  misc_feature (38)..(39): N is any nucleotide
  misc_feature (39)..(40): N is any nucleotide
  misc_feature (40)..(41): N is any nucleotide
  misc_feature (41)..(42): N is any nucleotide
  misc_feature (42)..(43): N is any nucleotide
  misc_feature (43)..(44): N is any nucleotide
  misc_feature (44)..(45): N is any nucleotide
  misc_feature (45)..(46): N is any nucleotide
  misc_feature (46)..(47): N is any nucleotide
  misc_feature (47)..(48): N is any nucleotide
  misc_feature (48)..(49): N is any nucleotide
  misc_feature (49)..(50): N is any nucleotide
  misc_feature (50)..(51): N is any nucleotide
  misc_feature (51)..(52): N is any nucleotide
  misc_feature (52)..(53): N is any nucleotide
  misc_feature (53)..(54): N is any nucleotide
  misc_feature (54)..(55): N is any nucleotide
  misc_feature (55)..(56): N is any nucleotide
  misc_feature (56)..(57): N is any nucleotide
  misc_feature (57)..(58): N is any nucleotide
  misc_feature (58)..(59): N is any nucleotide
  misc_feature (59)..(60): N is any nucleotide
  misc_feature (60)..(61): N is any nucleotide
  misc_feature (61)..(62): N is any nucleotide
  misc_feature (62)..(63): N is any nucleotide
  misc_feature (63)..(64): N is any nucleotide
  misc_feature (64)..(65): N is any nucleotide
tagggaagagaaggacatatgatnnnnnnnnnnnnnnnnnnnnnnnnnnnnnnnnnnnnnnnnnttgactagtacatgaccacttga